SEQUENCE LISTING (8 sequences)

SEQ ID NO: 1 - RNA, 21 nt, Artificial Sequence
  Synthetic
nnnnnnnnnnnnnnnnnnnnn

SEQ ID NO: 2 - RNA, 23 nt, Artificial Sequence
  Synthetic
auugguauucagugugaugacac

SEQ ID NO: 3 - DNA, 22 bp, Artificial Sequence
  Synthetic
cctaagatgagcgcaagttgaa

SEQ ID NO: 4 - DNA, 22 bp, Artificial Sequence
  Synthetic
ccacaggactagaacacctgct

SEQ ID NO: 5 - DNA, 22 bp, Artificial Sequence
  Synthetic
atggaagggaagtggttactgt

SEQ ID NO: 6 - DNA, 22 bp, Artificial Sequence
  Synthetic
gctttgtaggtgacctttggag

SEQ ID NO: 7 - DNA, 21 bp, Artificial Sequence
  Synthetic
aagcacctccgaaagtacgtg

SEQ ID NO: 8 - DNA, 23 bp, Artificial Sequence
  Synthetic
ctccagctctaccttacagttga

(19) United States
(12) Patent Application Publication
(10) Pub. No.: US 2013/0017167 A1
(43) Pub. Date: Jan. 17, 2013

(54) HYDROPHOBIC BLOCK CONJUGATED THERAPEUTIC AGENTS

(71) Applicants: Sean D. Monahan, Lake Forest Park, WA (US); Paul H. Johnson, Snohomish, WA (US); Charbel Diab, Seattle, WA (US); Priyadarsi De, Mohanpur (IN); Patrick S. Stayton, Seattle, WA (US); Allan S. Hoffman, Seattle, WA (US)

(72) Inventors: Sean D. Monahan, Lake Forest Park, WA (US); Paul H. Johnson, Snohomish, WA (US); Charbel Diab, Seattle, WA (US); Priyadarsi De, Mohanpur (IN); Patrick S. Stayton, Seattle, WA (US); Allan S. Hoffman, Seattle, WA (US)

(73) Assignee: University of Washington through its Center for Commercialization, Seattle, WA (US)

(21) Appl. No.: 13/509,557

(22) PCT Filed: Nov. 12, 2010
(86) PCT No.: PCT/US10/56565

§ 371 Date: Sep. 17, 2012

Related U.S. Application Data
(60) Provisional application No. 61/261,186, filed on Nov. 13, 2009.

(51) Int. Cl. C08F 220/34; C12N 5/07; A61K 31/795

(52) U.S. Cl. 424/78.23; 525/54.2; 435/375

(57) ABSTRACT

Provided herein are polymers having a therapeutic agent covalently coupled to a hydrophobic block thereof, as well as micelles and therapeutic compositions thereof.

### CROSS-REFERENCE TO RELATED APPLICATIONS

This application claims the benefit of U.S. Provisional Patent Application No. 61/261,186, filed Nov. 13, 2009 which is incorporated herein by reference in its entirety.

### BACKGROUND OF THE INVENTION

In certain instances, it is beneficial to provide therapeutic agents, such as polynucleotides (e.g., oligonucleotides) to living cells. In some instances, delivery of such polynucleotides to a living cell provides a therapeutic benefit.

### SUMMARY OF THE INVENTION

Among the various aspects of the invention is a composition comprising a block copolymer covalently conjugated to a polynucleotide. The block copolymer comprises a hydrophilic polymer block and a hydrophobic polymer block each comprising repeat units having chain atoms and pendant (side-chain) groups covalently coupled to the chain atoms. The polynucleotide is pendant and covalently coupled to the hydrophobic block. The hydrophobic block further comprises anionic repeat units having a population of pendant anions that varies in number in a pH dependant manner, the population being greater at pH 7.4 than at pH 5, wherein at least 90% of the repeat units of the hydrophilic and hydrophobic blocks are not the residues of amino acids linked by a peptidic bond.

Another aspect of the present invention is a composition comprising a block copolymer covalently conjugated to a polynucleotide. The block copolymer comprises a hydrophilic polymer block and a hydrophobic polymer block each comprising repeat units having chain atoms and pendant (side-chain) groups covalently coupled to the chain atoms. The polynucleotide is pendant and covalently coupled to the hydrophobic block. The hydrophobic block further comprises anionic repeat units having pendant groups in the form of carboxylic acid groups at pH 5 and carboxyl anions at pH 7.4.

A further aspect of the present invention is a composition containing a micelle. The micelle comprises a polymer having a hydrophobic block covalently conjugated to a polynucleotide. The hydrophobic polymer block comprises repeat units having chain atoms and pendant (side-chain) groups covalently coupled to the chain atoms, with the polynucleotide being pendant and covalently coupled to the hydrophobic block. The hydrophobic block further comprises anionic repeat units having a population of pendant anions that varies in number in a pH dependant manner, the population being greater at pH 7.4 than at pH 5, wherein at least 90% of the repeat units of the hydrophobic block are not the residues of amino acids linked by a peptidic bond.

A further aspect of the present invention is a composition containing a micelle. The micelle comprises a polymer having a hydrophobic block covalently conjugated to a polynucleotide. The hydrophobic polymer block comprises repeat units having chain atoms and pendant (side-chain) groups covalently coupled to the chain atoms, with the polynucleotide being pendant and covalently coupled to the hydrophobic block. The hydrophobic block further comprises anionic repeat units having pendant groups in the form of carboxylic acid groups at pH 5 and carboxyl anions at pH 7.4.

The present invention is further directed to a method for preparing a composition comprising a polymeric micelle and a polynucleotide associated with the micelle. The method comprises (i) dissolving a polymer having a hydrophobic block in a water miscible solvent, (ii) dissolving a polynucleotide in an aqueous solution and (iii) combining the two to covalently conjugate the polynucleotide to the hydrophobic block of the polymer and form the micelle.

The present invention is further directed to a pharmaceutical composition comprising the polymers or micelles of the present invention and a pharmaceutically acceptable excipient.

The present invention is further directed to the use of a composition comprising the polymers or micelles of the present invention in the manufacture of a medicament.

The present invention is further directed to a method for intracellular delivery of a polynucleotide. The method comprises contacting a composition comprising the polymers or micelles of the present invention with a cell surface in a medium at a first pH; introducing the composition into an endosomal membrane within the cell through endocytosis; and destabilizing the endosomal membrane, whereby the composition or the polynucleotide is delivered to the cytosol of the cell.

The present invention is further directed to a method for modulating the activity of an intracellular target in a cell. The method comprises delivering a polynucleotide to the cytosol of a cell using a composition comprising the polymers or micelles of the present invention, and allowing the polynucleotide to interact with the intracellular target, whereby the activity of the intracellular target is modulated.

Other aspects of the invention will be, in part apparent, and in part pointed out, hereinafter.

### ABBREVIATIONS AND DEFINITIONS

The following definitions and methods are provided to better define the present invention and to guide those of ordinary skill in the art in the practice of the present invention. Unless otherwise noted, terms are to be understood according to conventional usage by those of ordinary skill in the relevant art.

When introducing elements of the present invention or the preferred embodiment(s) thereof, the articles “a,” “an,” “the” and “said” are intended to mean that there are one or more of the elements. The terms “comprising”, “including” and “having” are intended to be inclusive and mean that there may be additional elements other than the listed elements.

Aliphatic: unless otherwise indicated, “aliphatic” or “aliphatic group” means an optionally substituted, non-aromatic hydrocarbon moiety. The moiety may be, for example, linear, branched, or cyclic (e.g., mono- or polycyclic such as fused, bridging, or spiro-fused polycyclic), or a combination thereof. Unless otherwise specified, aliphatic groups contain 1-20 carbon atoms.

Alkyl: unless otherwise indicated, the alkyl groups described herein are preferably lower alkyl containing from one to eight carbon atoms in the principal chain and up to 20 carbon atoms. They may be linear, branched or cyclic and include methyl, ethyl, propyl, butyl, hexyl and the like.

Amino: unless otherwise indicated, the term “amino” as used herein alone or as part of another group denotes the moiety —NR₁R₂ wherein R₁ and R₂ are independently hydrogen, hydrocarbyl, substituted hydrocarbyl or heterocyclo.

Amide or Amido: unless otherwise indicated, the “amide” or “amido” moieties represent a group of the formula —CONR₁R₂ wherein R₁ and R₂ are as defined in connection with the term “amino.” “Substituted amide,” for example, refers to a group of formula —CONR₁R₂ wherein at least one of R₁ and R₂ are other than hydrogen. “Unsubstituted amido,” for example, refers to a group of formula —CONR₁R₂, wherein R₁ and R₂ are both hydrogen.

Anionic Monomer, Anionic Monomeric Unit or Anionic Repeat Unit: unless otherwise indicated, an “anionic monomer,” “anionic monomeric unit” or “anionic repeat unit” is a monomer or a monomeric or repeat unit (the terms “monomeric unit” and “repeat unit” being used interchangeably) bearing a group that is present in an anionic charged state or in a non-charged state, but in the non-charged state is capable of becoming anionic charged, e.g., upon removal of an electrophile (e.g., a proton (H⁺), for example in a pH dependent manner). In certain instances, the group is substantially negatively charged at an approximately physiological pH but undergoes protonation and becomes substantially neutral at a weakly acidic pH. The non-limiting examples of such groups include carboxyl groups, barbituric acid and derivatives thereof, xanthine and derivatives thereof, boronic acids, phosphinic acids, phosphonic acids, sulfinic acids, phosphates, and sulfonamides.

Anionic species: unless otherwise indicated, an “Anionic species” is a group, residue or molecule that is present in an anionic charged or non-charged state, but in the non charged state is capable of becoming anionic charged, e.g., upon removal of an electrophile (e.g., a proton (H⁺), for example in a pH dependent manner). In certain instances, the group, residue or molecule is substantially negatively charged at an approximately physiological pH but undergoes protonation and becomes substantially neutral at a weakly acidic pH.

Aryl: unless otherwise indicated, the term “aryl” or “aryl group” refers to optionally substituted monocyclic, bicyclic, and tricyclic ring systems having a total of five to fourteen ring members, wherein at least one ring in the system is aromatic and wherein each ring in the system contains three to seven ring members. The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

Attached: unless otherwise indicated, two moieties or compounds are “attached” if they are held together by any interaction including, by way of example, one or more covalent bonds, one or more non-covalent interactions (e.g., ionic bonds, static forces, van der Waals interactions, combinations thereof, or the like), or a combination thereof.

Block Copolymer: unless otherwise indicated, a “block copolymer” comprises two or more homopolymer or copolymer subunits linked by covalent bonds. Block copolymers with two or three distinct blocks are called diblock copolymers and triblock copolymers, respectively. A schematic generalization of a diblock copolymer is represented by the formula [AₐB_{b}C_{c} . . . ]ₘ−[XₓY_{y}Z_{z} . . . ]ₙ, wherein each letter stands for a constitutional or monomeric unit, and wherein each subscript to a constitutional unit represents the mole fraction of that unit in the particular block, the three dots indicate that there may be more (there may also be fewer) constitutional units in each block and m and n indicate the molecular weight of each block in the diblock copolymer. As suggested by the schematic, in some instances, the number and the nature of each constitutional unit is separately controlled for each block. The schematic is not meant and should not be construed to infer any relationship whatsoever between the number of constitutional units or the number of different types of constitutional units in each of the blocks. Nor is the schematic meant to describe any particular number or arrangement of the constitutional units within a particular block. In each block the constitutional units may be disposed in a purely random, an alternating random, a regular alternating, a regular block or a random block configuration unless expressly stated to be otherwise. A purely random configuration, for example, may have the non-limiting form: x-x-y-z-x-y-y-z-y-z-z-z . . . . A non-limiting, exemplary alternating random configuration may have the non-limiting form: x-y-x-z-y-x-y-z-y-x-z . . . , and an exemplary regular alternating configuration may have the non-limiting form: x-y-z-x-y-z-x-y-z . . . . An exemplary regular block configuration may have the following non-limiting configuration: . . . x-x-x-y-y-y-z-z-z-x-x-x . . . , while an exemplary random block configuration may have the non-limiting configuration: . . . x-x-x-z-z-x-x-y-y-y-y-z-z-z-x-x-z-z-z- . . . . In a gradient polymer, the content of one or more monomeric units increases or decreases in a gradient manner from the alpha-end of the polymer to the omega-end. In none of the preceding generic examples is the particular juxtaposition of individual constitutional units or blocks or the number of constitutional units in a block or the number of blocks meant nor should they be construed as in any manner bearing on or limiting the actual structure of block copolymers forming a micelle described herein. As used herein, the brackets enclosing the constitutional units are not meant and are not to be construed to mean that the constitutional units themselves form blocks. That is, the constitutional units within the square brackets may combine in any manner with the other constitutional units within the block, i.e., purely random, alternating random, regular alternating, regular block or random block configurations. The block copolymers described herein are, optionally, alternate, gradient or random block copolymers. In some embodiments, the block copolymers are dendrimer, star or graft copolymers.

Cationic Monomer, Cationic Monomeric Unit or Cationic Repeat Unit: unless otherwise indicated, an “cationic monomer,” “cationic monomeric unit” or “cationic repeat unit” is a monomer or a monomeric or repeat unit (the terms “monomeric unit” and “repeat unit” being used interchangeably) bearing a cation or a moiety capable of having a cationic charge upon addition of an electrophile (e.g., a proton (H+)).

Chargeable species, Chargeable Group, or Chargeable Monomeric Unit: unless otherwise indicated, a “chargeable species,” “chargeable group” or “chargeable monomeric unit” is a species, group or monomeric unit in either a charged or non-charged state. In certain instances, a “chargeable monomeric unit” is one that can be converted to a charged state (either an anionic or cationic charged state) by the addition or removal of an electrophile (e.g., a proton (H⁺), for example in a pH dependent manner). The use of any of the terms “chargeable species”, “chargeable group”, or “chargeable monomeric unit” includes the disclosure of any other of a “chargeable species”, “chargeable group”, or “chargeable monomeric unit” unless otherwise stated. A “chargeable species” that is “charged or chargeable to an anion” or “charged or chargeable to an anionic species” is a species or group that is either in an anionic charged state or non-charged state, but in the non-charged state is capable of being converted to an anionic charged state, e.g., by the removal of an electrophile, such as a proton (H⁺). A “chargeable species” that is “charged or chargeable to a cation” or “charged or chargeable to a cationic species” is a species or group that is either in an cationic charged state or non-charged state, but in the non-charged state is capable of being converted to a cationic charged state, e.g., by the addition of an electrophile, such as a proton (H⁺). “Chargeable monomeric units” described herein are used interchangeably with “chargeable monomeric residues”.

Copolymer: unless otherwise indicated, the term “copolymer” signifies that the polymer is the result of polymerization of two or more different monomers.

Critical Micelle Concentration and CMC: unless otherwise indicated, the “critical micelle concentration” or “CMC” is the concentration at which a micelle self-assembles. The CMC can be determined by well known methods such as the uptake of a hydrophobic probe molecule (for example, a pyrene fluorescence assay).

Dicer Substrate: unless otherwise indicated, a “dicer substrate” is a substrate for the RNase III family member Dicer in cells, the substrate possessing at least about 25 base pair duplex RNA. Dicer substrates are cleaved to produce approximately 21 base pair duplex small interfering RNAs (siRNAs) that evoke an RNA interference effect resulting in gene silencing by mRNA knockdown.

Endosome Disruptive & Endosomolytic: unless otherwise indicated, a composition is “endosome disruptive,” also sometimes referred to as “endosomolytic,” if the effect of the composition upon the endosome is to increase the permeability of the endosomal membrane.

Heteroalkyl: unless otherwise indicated, the term “heteroalkyl” means an alkyl group wherein at least one of the backbone carbon atoms is replaced with a heteroatom.

Heteroaryl: unless otherwise indicated, the term “heteroaryl” means an aryl group wherein at least one of the ring members is a heteroatom, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto (i.e., ═O), hydroxy, protected hydroxy, acyl, acyloxy, alkoxy, alkenoxy, alkynoxy, aryloxy, halogen, amido, amino, nitro, cyano, thiol, ketals, acetals, esters and ethers.

Heteroatom: unless otherwise indicated, the term “heteroatom” means an atom other than hydrogen or carbon, such as an oxygen, sulfur, nitrogen, phosphorus, boron, arsenic, selenium or silicon atom.

Heterocyclo: unless otherwise indicated, the terms “heterocyclo” and “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclo include heteroaromatics such as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

Heterohydrocarbyl: unless otherwise indicated, the term “heterohydrocarbyl” means a hydrocarbyl group wherein at least one of the chain carbon atoms is replaced with a heteroatom.

Hydrocarbon or Hydrocarbyl: unless otherwise indicated, the terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms

Hydrophobic: unless otherwise indicated, the terms “hydrophobic” and “hydrophobicity” are terms of art describing a physical property of a composition measured by the free energy of transfer of the composition between a non-polar solvent and water (Hydrophobicity regained. Karplus P. A., Protein Sci., 1997, 6: 1302-1307.). The hydrophobicity of a composition can be measured by its logP value, the logarithm of a partition coefficient (P), which is defined as the ratio of concentrations of a compound in the two phases of a mixture of two immiscible solvents, e.g., octanol and water. Experimental methods of determination of hydrophobicity as well as methods of computer-assisted calculation of logP values are known to those skilled in the art. Hydrophobic species of the present invention include but are not limited to aliphatic, heteroaliphatic, aryl, and heteroaryl groups.

Hydrophobic Core: unless otherwise indicated, a “hydrophobic core” comprises hydrophobic moieties. In certain instances, a “hydrophobic core” is substantially non-charged (e.g., the charge is substantially net neutral).

Hydrophobic Repeat Unit: unless otherwise indicated, a “hydrophobic repeat unit” or a “hydrophobic monomeric unit” is a repeat unit or monomeric unit of a polymer possessing a hydrophobic substituent.

Hydrophobic Species: unless otherwise indicated, the terms “hydrophobic species” and “hydrophobic-enhancing moiety” is a moiety such as a substituent, residue or a group which, when covalently attached to a molecule, such as a monomer or a polymer, increases the molecule's hydrophobicity.

Inhibition, Silencing, Attenuation or Knock-Down: unless otherwise indicated, the terms “inhibition,” “silencing,” and “attenuation” as used herein refer to a measurable reduction in expression of a target mRNA or the corresponding protein as compared with the expression of the target mRNA or the corresponding protein in the absence of a knockdown agent. “Knockdown,” or the reduction in expression of the target mRNA or the corresponding protein, can be assessed by measuring the mRNA levels using techniques well known in the art such as quantitative polymerase chain reaction (qPCR) amplification, RNA solution hybridization, nuclease protection, northern blotting and hybridization, and gene expression monitoring with a microarray; and in the case of proteins by techniques well known in the art such as SDS-PAGE, antibody binding, western blot analysis, immunoprecipitation, radioimmunoassay or enzyme-linked immunosorbent assay (ELISA), fluorescence activated cell analysis and immunocytochemistry.

Inhibit gene expression: unless otherwise indicated, the phrase “inhibit gene expression” means to cause any measurable reduction in the amount of an expression product of the gene. The expression product can be an RNA transcribed from the gene (e.g., an mRNA) and/or a polypeptide translated from an mRNA transcribed from the gene. The level of expression may be determined using standard techniques for measuring mRNA or protein.

Membrane Destabilizing Polymer or Membrane Destabilizing Block: unless otherwise indicated, a “membrane destabilizing polymer” or a “membrane destabilizing block” can directly or indirectly elicit a change (e.g., a permeability change) in a cellular membrane structure (e.g., an endosomal membrane) so as to permit an agent (e.g., polynucleotide), in association with or independent of a micelle (or a constituent polymer thereof), to pass through such membrane structure, for example, to enter a cell or to exit a cellular vesicle (e.g., an endosome). A membrane destabilizing polymer can be (but is not necessarily) a membrane disruptive polymer. A membrane disruptive polymer can directly or indirectly elicit lysis of a cellular vesicle or otherwise disrupt a cellular membrane (e.g., as observed for a substantial fraction of a population of cellular membranes). Generally, membrane destabilizing or membrane disruptive properties of polymers or micelles can be assessed by various means. In one non-limiting approach, a change in a cellular membrane structure can be observed by assessment in assays that measure (directly or indirectly) release of an agent (e.g., polynucleotide) from cellular membranes (e.g., endosomal membranes), for example, by determining the presence or absence of such agent, or an activity of such agent, in an environment external to such membrane. Another non-limiting approach involves measuring red blood cell lysis (hemolysis), e.g., as a surrogate assay for a cellular membrane of interest. Such assays may be done at a single pH value or over a range of pH values.

Micelle: unless otherwise indicated, a “micelle” is a particle comprising a core and a hydrophilic shell, wherein the core is held together at least partially, predominantly or substantially through hydrophobic interactions. The micelle may be, for example, a multi-component, nanoparticle comprising at least two domains, the inner domain or core, and the outer domain or shell. Micelle particles described herein may have any suitable or desired diameter, e.g., of less than 1000 nanometers (nm). In general, the micelle should have dimensions small enough to allow their uptake by eukaryotic cells. Typically, the micelle has a longest lateral dimension, also known as the diameter, of 200 nm or less. In some embodiments, the micelle has a diameter of 100 nm or less. Smaller micelles, e.g. having diameters of about 10 nm to about 200 nm, about 20 nm to about 100 nm, or 50 nm or less, e.g., 5 nm-30 nm, are used in some embodiments. Micelle particle size can be determined in any manner, including, but not limited to, by gel permeation chromatography (GPC), dynamic light scattering (DLS), electron microscopy techniques (e.g., TEM), and other methods.

Non-Charged Repeat Units: unless otherwise indicated, a “non-charged repeat unit” is a repeat unit that is neither an anionic repeat unit or a cationic repeat unit.

Nucleoside: unless otherwise indicated, the term “nucleoside” is used to describe a composition comprising a monosaccharide and a base. The monosaccharide includes but is not limited to pentose and hexose monosaccharides. The monosaccharide also includes monosaccharide mimetics and monosaccharides modified by substituting hydroxyl groups with halogens, methoxy, hydrogen or amino groups, or by esterification of additional hydroxyl groups.

Nucleotide: unless otherwise indicated, the term “nucleotide,” in its broadest sense, refers to any compound and/or substance that is or can be incorporated into a polynucleotide (e.g., oligonucleotide) chain. In some embodiments, a nucleotide is a compound and/or substance that is or can be incorporated into a polynucleotide (e.g., oligonucleotide) chain via a phosphodiester linkage. In some embodiments, “nucleotide” refers to individual nucleic acid residues (e.g. nucleotides and/or nucleosides). In certain embodiments, “at least one nucleotide” refers to one or more nucleotides present; in various embodiments, the one or more nucleotides are discrete nucleotides, are non-covalently attached to one another, or are covalently attached to one another. As such, in certain instances, “at least one nucleotide” refers to one or more polynucleotide (e.g., oligonucleotide). In some embodiments, a polynucleotide is a polymer comprising two or more nucleotide monomeric units.

Oligonucleotide: unless otherwise indicated, the term “oligonucleotide” refers to a polymer comprising 7-200 nucleotide monomeric units. In some embodiments, “oligonucleotide” encompasses single and or/double stranded RNA as well as single and/or double-stranded DNA. Furthermore, the terms “nucleotide”, “nucleic acid,” “DNA,” “RNA,” and/or similar terms include nucleic acid analogs, i.e., analogs having a modified backbone, including but not limited to peptide nucleic acids (PNA), locked nucleic acids (LNA), phosphono-PNA, morpholino nucleic acids, or nucleic acids with modified phosphate groups (e.g., phosphorothioates, phosphonates, 5′-N-phosphoramidite linkages). Nucleotides can be purified from natural sources, produced using recombinant expression systems and optionally purified, chemically synthesized, etc. In some embodiments, a nucleotide is or comprises a natural nucleoside phosphate (e.g. adenosine, thymidine, guanosine, cytidine, uridine, deoxyadenosine, deoxythymidine, deoxyguanosine, and deoxycytidine phosphate). In some embodiments, the base includes any bases occurring naturally in various nucleic acids as well as other modifications which mimic or resemble such naturally occurring bases. Nonlimiting examples of modified or derivatized bases include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, wybutoxosine, pseudouracil, queosine, 2-thiocytosine, 5-methyl-2-thiouracil, 2-thiouracil, 4-thiouracil, 5 methyluracil, uracil-5-oxyacetic acid methylester, uracil-5-oxyacetic acid, 5-methyl-2-thiouracil, 3-(3-amino-3-N-2-carboxypropyl)uracil, 2-aminoadenine, pyrrolopyrimidine, and 2,6-diaminopurine. Nucleoside bases also include universal nucleobases such as difluorotolyl, nitroindolyl, nitropyrrolyl, or nitroimidazolyl. Nucleotides also include nucleotides which harbor a label or contain abasic, i.e., lacking a base, monomers. A nucleic acid sequence is presented in the 5′ to 3′ direction unless otherwise indicated. A nucleotide can bind to another nucleotide in a sequence-specific manner through hydrogen bonding via Watson-Crick base pairs. Such base pairs are said to be complementary to one another. An oligonucleotide can be single stranded, double-stranded or triple-stranded.

Oligonucleotide gene expression modulator: as used herein, an “oligonucleotide gene expression modulator” is an oligonucleotide agent capable of inducing a selective modulation of gene expression in a living cell by mechanisms including but not limited to an antisense mechanism or by way of an RNA interference (RNAi)-mediated pathway which may include (i) transcription inactivation; (ii) mRNA degradation or sequestration; (iii) transcriptional inhibition or attenuation or (iv) inhibition or attenuation of translation. Oligonucleotide gene expression modulators include, regulatory RNA (including virtually any regulatory RNA) such as, but not limited to, antisense oligonucleotides, miRNA, sRNA, RNAi, shRNA, aptamers and any analogs or precursors thereof.

Oligonucleotide Knockdown Agent: unless otherwise indicated, an “oligonucleotide knockdown agent” is an oligonucleotide species which can inhibit gene expression by targeting and binding an intracellular nucleic acid in a sequence-specific manner. Non-limiting examples of oligonucleotide knockdown agents include sRNA, miRNA, shRNA, dicer substrates, antisense oligonucleotides, decoy DNA or RNA, antigene oligonucleotides and any analogs and precursors thereof.

pH Dependent, Membrane-Destabilizing: unless otherwise indicated, a “pH dependent, membrane-destabilizing” group or block is a group or block that is at least partially, predominantly, or substantially hydrophobic and is membrane destabilizing in a pH dependent manner. In certain instances, a pH dependent membrane destabilizing polymer block is a hydrophobic polymeric segment of a block copolymer and/or comprises a plurality of hydrophobic species; and comprises a plurality of chargeable species. In some embodiments, the chargeable species is anionic. In some embodiments, the anionic chargeable species is anionic at about neutral pH. In further or alternative embodiments, the anionic chargeable species is non-charged at a lower, e.g., endosomal pH. In some embodiments, the membrane destabilizing chargeable hydrophobe comprises a plurality of cationic species. The pH dependent membrane-destabilizing chargeable hydrophobe comprises a non-peptidic and non-lipidic polymer backbone. For example, a pH dependent, membrane-destabilizing block may possess anionic repeat units the substituents of which are predominantly ionized (anions) at one pH, e.g., pH 7.4, and predominantly neutral at a lesser pH, e.g., pH 5.0 whereby the pH dependent, membrane-destabilizing group or block becomes increasingly hydrophobic as a function of the drop in pH from 7.4 to 5.0.

RNAi agent: unless otherwise indicated, the term “RNAi agent” refers to an oligonucleotide which can mediate inhibition of gene expression through an RNAi mechanism and includes, but is not limited to, siRNA, microRNA (miRNA), short hairpin RNA (shRNA), asymmetrical interfering RNA (aiRNA), dicer substrate and the precursors thereof.

RNA interference (RNAi): unless otherwise indicated, the term “RNA interference” or “RNAi” refers to sequence-specific inhibition of gene expression and/or reduction in target mRNA and protein levels mediated by an at least partially double-stranded RNA, which also comprises a portion that is substantially complementary to a target RNA.

Short hairpin RNA (shRNA): unless otherwise indicated, “short hairpin RNA” or “shRNA” refers to an oligonucleotide having at least two complementary portions hybridized or capable of hybridizing with each other to form a double-stranded (duplex) structure and at least one single-stranded portion.

Short interfering RNA (siRNA): unless otherwise indicated, “short interfering RNA” or “siRNA” refers to an RNAi agent that is approximately 15-50 base pairs in length and optionally further comprises zero to two single-stranded overhangs. One strand of the siRNA includes a portion that hybridizes with a target RNA in a complementary manner. In some embodiments, one or more mismatches between the siRNA and the targeted portion of the target RNA may exist. In some embodiments, siRNAs mediate inhibition of gene expression by causing degradation of target transcripts.

Substantially Non-Charged Block: unless otherwise indicated, the term “substantially non-charged block” means a polymeric block that has a neutral charge, or a near neutral charge. For example, less than about 10 mole % of the repeat units in a block will be anionic, cationic or zwitterionic repeat units in a substantially non-charged block. By way of further example, less than about 5 mole % of the repeat units in a block will be anionic, cationic or zwitterionic repeat units in a substantially non-charged block.

Substituted or Optionally Substituted: unless otherwise indicated, the term “substituted” and “optionally substituted” means that the referenced group is or may be substituted with one or more additional suitable group(s), which may be individually and independently selected from acetals, acyl, acyloxy, alkenoxy, alkoxy, alkylthio, alkynoxy, amido, amino, aryl, aryloxy, arylthio, carbonyl, carboxamido, carboxyl, cyano, esters, ethers, hydrocarbyl, substituted hydrocarbyl, heterohydrocarbyl, substituted heterohydroalkyl, cycloalkyl, halogen, heteroalicyclic, heteroaryl, hydroxy, isocyanato, isothiocyanato, ketals, keto, mercapto, nitro, perhaloalkyl, silyl, sulfonamido, sulfonyl, thiocarbonyl, thiocyanato, thiol, and/or the protected derivatives thereof.

Therapeutic agent: unless otherwise indicated, the phrase “therapeutic agent” refers to any agent that, when administered to a subject, organ, tissue, or cell has a therapeutic effect and/or elicits a desired biological and/or pharmacological effect.

Therapeutically effective amount: unless otherwise indicated, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition.

Zwitterionic Monomer, Zwitterionic Monomeric Unit or Zwitterionic Repeat Unit: unless otherwise indicated, a “zwitterionic monomer,” “zwitterionic monomeric unit” or “zwitterionic repeat unit” is a monomer or a monomeric or repeat unit (the terms “monomeric unit” and “repeat unit” being used interchangeably) bearing (i) a cation or a moiety capable of having a cationic charge upon addition of an electrophile (e.g., a proton (H+)) and (ii) an anion or a moiety capable of having an anionic charge upon removal of an electrophile (e.g., a proton (H+)).

### DETAILED DESCRIPTION OF THE PREFERRED EMBODIMENTS

One aspect of the present invention is a composition comprising a particle containing a therapeutic agent covalently coupled to a hydrophobic block of a polymer, sometimes also referred to herein as the first hydrophobic block or more simply, the hydrophobic block. Depending upon the therapeutic agent, the composition and length of any linker coupling the therapeutic agent to the first hydrophobic block, and the composition and size of the first hydrophobic block, the therapeutic agent may be accommodated in the particle core, or alternatively, outside the particle core.

In one preferred embodiment, the particle comprises a hydrophobic core within a hydrophilic shell. The hydrophobic core and hydrophilic shell, in turn, may be formed by the self-assembly and aggregation of molecules having hydrophilic “heads” and hydrophobic “tails” whereby, in aqueous solution the hydrophilic shell comprises the hydrophilic heads of the molecules and the hydrophobic core comprises the hydrophobic tails of the molecules. In one preferred embodiment, the particle is a micelle having a critical micelle concentration, or “CMC”, of about 0.2 μg/ml to about 100 μg/ml.

The polymer to which the therapeutic agent is covalently coupled comprises, at a minimum, a hydrophobic polymeric block having a polymer backbone and pendant, i.e., side chain, groups. The therapeutic agent is pendant and covalently coupled to the backbone of the hydrophobic block.

In one preferred embodiment, the polymer to which the therapeutic agent is covalently coupled is a multiblock copolymer. Stated differently, in one embodiment, the polymer has at least two compositionally distinct polymer blocks. For example, the polymer may be a diblock polymer having two compositionally distinct polymer blocks. By way of further example, the polymer may be a triblock polymer having three compositions distinct polymer blocks. By way of further example, the polymer may have four or more compositionally distinct polymer blocks.

Advantageously, when the polymer is a multiblock copolymer, each of the blocks may possess somewhat different characteristics or provide a somewhat different function to the polymer. For example, in one embodiment, the polymer is a multiblock polymer comprising a hydrophilic polymeric block, sometimes referred to herein as the “first hydrophilic block,” or simply the “hydrophilic block.” The hydrophilic block may be used, for instance, to contribute water solubility to the copolymer, to aid in micelle formation, to target the copolymer to a cellular or other biological target, to shield a therapeutic agent that is associated with the copolymer, or a combination of two or more thereof. Optionally, the polymer may contain a second, compositionally distinct hydrophilic block, sometimes referred to herein as the “second hydrophilic block,” that complements the first hydrophilic block by providing a property or function not provided by the first hydrophilic block; for example, the second hydrophilic block may be used to provide means for attaching a therapeutic agent, contribute water solubility to the copolymer, aid in micelle formation, target the copolymer to a cellular or other biological target, shield a therapeutic agent that is associated with the copolymer, or a combination of two or more thereof. Alternatively, or additionally, the polymer may comprise, in addition to the hydrophobic block to which the therapeutic agent is conjugated, at least one additional compositionally distinct hydrophobic block. The additional, or second hydrophobic block may be used to decrease the water solubility of the copolymer, aid in micelle formation, destabilize a cellular membrane or other biological target, or a combination of two or more thereof. The copolymer may optionally possess further additional polymeric blocks that amplify the function of the copolymers of the present invention, or which introduce other functionalities or properties to the copolymer.

Regardless of the number of blocks, it is generally preferred that the number average molecular weight of the polymeric blocks, in combination, be about 5,000 to about 100,000 daltons. Additionally, in one embodiment, the Zeta potential of a polymeric solution containing a block polymer of the present invention (without an associated therapeutic agent such as a polynucleotide) is between ±6 mV (millivolt). In one preferred embodiment, the Zeta potential of the polymeric solution (without an associated therapeutic agent such as a polynucleotide) is between ±5 mV. In one preferred embodiment, the Zeta potential of the polymeric solution (without an associated therapeutic agent such as a polynucleotide) is between ±2 mV.

In general, the properties of the blocks of the polymer of the present invention may be tuned by selection of the monomeric residues constituting each of the blocks or the relative mole fractions. For example, when the polymer is a block copolymer comprising a hydrophilic block in addition to the first hydrophobic block, the difference in hydrophilicity may be achieved by the polymerization of different sets of monomers for the hydrophilic and hydrophobic blocks; alternatively, the same set of monomers may be used for the two blocks, but the hydrophilic block will contain a relatively greater percentage of hydrophilic monomeric residues to provide the hydrophilic block with an overall hydrophilic character whereas the hydrophobic block will contain a relatively greater percentage of hydrophobic monomeric residues to provide the hydrophobic block with an overall hydrophobic character. Similarly, when the polymer comprises first and second hydrophilic blocks, the difference between the two may be achieved by the polymerization of different sets of monomers for the first and second hydrophilic blocks; alternatively, the same set of monomers may be used for the two blocks, but the mole fraction of the monomers will be varied to provide the first and second hydrophilic blocks with different functions or properties. Similarly, when the polymer comprises first and second hydrophobic blocks, the difference between the two may be achieved by the polymerization of different sets of monomers for the first and second hydrophobic blocks; alternatively, the same set of monomers may be used for the two blocks, but the mole fraction of the monomers will be varied to provide the first and second hydrophobic blocks with different functions or properties.

When the polymer comprises at least two compositionally distinct polymer blocks, the blocks may be covalently coupled via a polymeric or non-polymeric linking moiety. For example, when the polymer comprises a hydrophilic block in addition to the hydrophobic block, the hydrophilic block may be coupled to the hydrophobic block by a series of amino acid residues, saccharide residues, nucleic acid residues, etc., to introduce a cleavage point or other functionality between the respective blocks. By way of further example, the blocks may be covalently coupled by a cleavable moiety such as a disulfide, a hydrazide, an ester, an acetal, or a phosphodiester linking moiety. In general, it is presently preferred that the hydrophilic block, when present, be immediately adjacent to the hydrophobic block.

In general, it is preferred that at least one of the polymer block(s) of the polymer of the present invention be a copolymer block and, still more preferred that at least one of polymer block(s) be a random copolymer block. Thus, for example, it is generally preferred that the hydrophobic block be a random copolymer comprising two or more compositionally distinct monomeric residues. When the polymer additionally comprises a hydrophilic block, the hydrophilic block or the hydrophobic block may be a random copolymer block comprising two or more compositionally distinct monomeric residues; in this embodiment, the hydrophilic block, the hydrophobic block or both of the hydrophobic and hydrophilic blocks may be a random copolymer block comprising two or more compositionally distinct monomeric residues. Additionally, when the polymer comprises at least two compositionally distinct hydrophilic blocks, the hydrophobic block or at least one of the hydrophilic blocks may be a random copolymer block comprising two or more compositionally distinct monomeric residues; in this embodiment, at least one of the hydrophilic blocks, the hydrophobic block or each of the hydrophobic and hydrophilic blocks may be a random copolymer block comprising two or more compositionally distinct monomeric residues. Additionally, when the polymer comprises at least two compositionally distinct hydrophobic blocks, at least one of the hydrophobic blocks may be a random copolymer block comprising two or more compositionally distinct monomeric residues. Additionally, when the polymer comprises at least two compositionally distinct hydrophobic blocks and a hydrophilic block, at least one of the hydrophobic blocks or the hydrophilic block may be a random copolymer block comprising two or more compositionally distinct monomeric residues. In some embodiments, the first (and/or second) hydrophilic block may be a homopolymer block(s). For example, in one embodiment, the polymer comprises a first hydrophilic block, the first hydrophilic block is a homopolymer and the first hydrophobic block is a random copolymer block. By way of further example, in one embodiment, the polymer comprises first and second hydrophilic blocks, the first and second hydrophilic blocks are compositionally distinct homopolymers and the hydrophobic block is a random copolymer. By way of further example, in one embodiment, the polymer comprises first and second hydrophilic blocks, one of the first and second hydrophilic blocks is a homopolymer and the other hydrophilic block and the hydrophobic block are random copolymer blocks. By way of yet further example, in one embodiment, each of the polymer blocks, hydrophilic or hydrophobic are compositionally distinct random copolymers.

The hydrophobic block and, optionally, other polymeric blocks, when present, comprise chain atoms and pendant groups covalently coupled to the chain atoms. Preferably, the chain atoms are carbon or a combination of (i) carbon and (ii) sulfur and/or oxygen atoms. Thus, for example, the repeat units of the hydrophobic polymer block, and optionally one or more other polymeric blocks, when present, are independently selected from the group consisting of substituted alkylene, substituted alkylene glycol, and substituted alkylene thioglycol repeat units, and combinations thereof. In one preferred embodiment, the chain atoms of the hydrophobic polymer block, and optionally one or more other polymeric blocks, when present, are carbon. In another embodiment, the polymer comprises a hydrophobic block and a hydrophilic block and the chain atoms of the hydrophobic and hydrophilic blocks are independently carbon or a combination of carbon, oxygen and sulfur atoms. Independent of the selection of the chain atoms, the pendant groups are preferably selected from the group consisting of hydrogen, hydrocarbyl, substituted hydrocarbyl, substituted carbonyl and heterocyclo.

In a preferred embodiment, the hydrophobic block of the polymer of the present invention is prepared by a method other than by stepwise coupling approaches involving a sequence of multiple individual reactions (e.g., such as known in the art for peptide synthesis or for oligonucleotide synthesis). For example, in one embodiment, the hydrophobic block is formed by chain-growth polymerization, sometimes referred to as addition polymerization. By way of further example, in one embodiment, the polymer comprises at least one other polymer block in addition to the hydrophobic block and at least two of the polymer blocks are formed by chain-growth polymerization. By way of further example, in one embodiment, each of the polymer blocks is formed by chain-growth polymerization.

Although each of the blocks may contain repeat units linked by amide bonds, formed for example, by the condensation reaction of amino acids or by the condensation reaction of species other than amino acids (e.g., diamines and dicarboxylic acids), it is generally preferred that the substantial majority of the residues constituting the hydrophobic block and the hydrophilic block (when present) not be peptidic. Stated differently, it is generally preferred that the substantial majority of the residues of the hydrophobic block and the hydrophilic block, when present, be other than amino acid residues linked by peptide bonds. For example, in one embodiment at least 90% of the residues constituting the hydrophilic block are other than amino acid residues linked by peptide bonds. By way of further example, in one embodiment the polymer comprises a second hydrophobic polymer block and at least 90% of the residues constituting the second hydrophobic block are other than amino acid residues linked by peptide bonds. By way of further example, in one embodiment the polymer comprises a hydrophilic block and at least 90% of the residues constituting the hydrophilic block are other than amino acid residues linked by peptide bonds. By way of further example, in one embodiment it is preferred that at least 90% of the residues constituting each of the blocks be other than amino acid residues linked by peptide bonds. Preferably, each block is a substantially non peptidic polymer (consists of a polymer other than an amino acid polymer).

In contrast, for clarity, notwithstanding and without prejudice to the foregoing, the targeting moieties and/or other biomolecular agents of the inventions can be an amino acid polymer (e.g., a peptide) or a nucleic acid polymer (e.g., an oligonucleotide) or a polysaccharide. In one preferred embodiment, peptides, saccharides, or nucleic acid residues are attached, as pendant groups, to the repeat units to increase water solubility, to provide shielding, to provide targeting, or as a therapeutic agents. or other hydrophilic or targeting groups are attached, as pendant groups to the repeat units. When attached as pendant groups, however, the peptides, saccharides, and nucleic acids do not provide peptide, glycosidic or phosphodiester bonds along the backbone of the polymer block which they would do if incorporated as repeat units.

Conveniently, the hydrophobic block and other blocks, when present, may be prepared from readily polymerizable monomers. For example, in one embodiment, the repeat units are residues of ethylenically unsaturated monomer(s). In another, the hydrophobic block and other blocks, when present, comprise repeat units independently derived from optionally substituted acrylic acid monomers, optionally substituted vinyl aryl monomers, optionally substituted acrylamide monomers, optionally substituted acrylate monomers and combinations thereof.

In one preferred embodiment, the hydrophobic block or one or more other blocks comprises repeat units of Formula 1

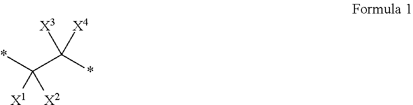

wherein * designates the point of attachment of the repeat unit of Formula 1 to other repeat units; each X¹ and X² is independently selected from the group consisting of hydrogen, hydrocarbyl, substituted hydrocarbyl, heterocyclo, and substituted carbonyl, provided, however, X¹ and X² are not, in the same repeat unit, selected from the group consisting of aryl, heteroaryl, heterosubstituted carbonyl, and combinations thereof; each X³ is independently hydrogen, alkyl or substituted alkyl, and each X⁴ is independently heterosubstituted carbonyl, aryl, or heteroaryl. For example, in one such embodiment, the hydrophobic block, and the hydrophilic block, when present, comprise repeat units corresponding to Formula 1 in which X⁴ is aryl or heteroaryl. In another such embodiment, the hydrophobic block, and the hydrophilic block, when present, comprise repeat units corresponding to Formula 1 in which X⁴ is —C(O)OX⁴⁰, —C(O)SX⁴⁰, or —C(O)NX⁴⁰X⁴¹, and X⁴⁰ and X⁴¹ are independently hydrogen, hydrocarbyl, substituted hydrocarbyl, heterohydrocarbyl, substituted heterohydrocarbyl, or heterocyclo. In another such embodiment, the polymer comprises a hydrophilic block and the hydrophobic and hydrophilic blocks comprise repeat units corresponding to Formula 1 in which X⁴ is —C(O)OX⁴⁰, —C(O)SX⁴⁰, or —C(O)NX⁴⁰X⁴¹, and X⁴⁰ and X⁴¹ are independently hydrogen, hydrocarbyl, substituted hydrocarbyl, heterohydrocarbyl, substituted heterohydrocarbyl, or heterocyclo. In another such embodiment, the polymer is a triblock polymer, comprising at least one hydrophilic block and at least one hydrophobic block, and each of the blocks of the triblock copolymer comprise repeat units corresponding to Formula 1 in which X⁴ is —C(O)OX⁴⁰, —C(O)SX⁴⁰, or —C(O)NX⁴⁰X⁴¹, and X⁴⁰ and X⁴¹ are independently hydrogen, hydrocarbyl, substituted hydrocarbyl, heterohydrocarbyl, substituted heterohydrocarbyl, or heterocyclo. In another such embodiment, the polymer is a multiblock polymer, comprising at least one hydrophilic block and at least one hydrophobic block, and each of the blocks of the multiblock copolymer comprise repeat units corresponding to Formula 1 in which X⁴ is —C(O)OX⁴⁰ or —C(O)NX⁴⁰X⁴¹ and X⁴⁰ and X⁴¹ are independently hydrogen, hydrocarbyl, substituted hydrocarbyl, heterohydrocarbyl, substituted heterohydrocarbyl, or heterocyclo.

Hydrophobic Block

In general, the hydrophobic block comprises, as a pendant group, a therapeutic agent such as a polynucleotide. The therapeutic agent may be a pendant group on the monomer prior to the polymerization and incorporation of the monomeric residue as a repeat unit. Alternatively, the monomer may contain a conjugatable pendant group that may be derivatized to covalently couple the therapeutic agent post-polymerization. In some instances, a conjugatable pendant group is a moiety bearing one or more reactive groups that can be used for post-polymerization introduction of additional functionalities via known in the art chemistries (such as the techniques described in Hermanson, G. T., Bioconjugate Techniques, 2nd ed. (Pierce Biotechnology, Thermo Fisher Scientific, Rockford, Ill.). Academic Press (an imprint of Elsevier): London, Amsterdam, Burlington, San Diego. 2008. 1202 pp.), for example, “click” chemistry (for example of “click” reactions, see Wu, P.; Fokin, V. V. Catalytic Azide-Alkyne Cycloaddition: Reactivity and Applications. Aldrichim. Acta, 2007, 40, 7-17). In certain embodiments, conjugatable side chains provided herein comprise one or more of any suitable electrophilic or nucleophilic functional group, such as but not limited to N-hydroxysuccinimide (NHS)ester, HOBt (1-hydroxybenzotriazole) ester, p-nitrophenyl ester, tetrafluorophenyl ester, pentafluorophenyl ester, pyridyl disulfide group, maleimide, aldehyde, ketone, anhydride, thiol, amine, hydroxyl, alkyl halide, or the like.

The hydrophobic block additionally comprises repeat units having pendant groups selected from the group consisting of hydrophobic species, cationic species, anionic species, zwitterionic species and non-charged hydrophilic species. In general, different repeat units in the hydrophobic block may independently comprise one or more of these species. For example, some repeat units may comprise only hydrophobic species. Other repeat units may comprise only cationic species. Other repeat units may comprise only zwitterionic species. Other repeat units may comprise only anionic species. Still others may comprise an anionic species and a hydrophobic species on the same, individual repeat unit. Still others may comprise a cationic species and a hydrophobic species on the same, individual repeat unit.

In one embodiment, the hydrophobic block comprises a population of repeat units having pendant hydrophobic species (used interchangeably herein with “hydrophobicity enhancers”). Exemplary hydrophobic species include optionally-substituted alkyl, alkaryl such as aryl-alkyl, alkyl-aryl, and alkyl-aryl-alkyl, heteroalkyl, aryl, and heteroaryl. In specific embodiments, the hydrophobic species is optionally substituted alkyl, alkaryl, or aryl. In some embodiments, hydrophobic block comprises a population of monomeric residues having pendant hydrophobic species selected from (C₂-C₈) alkyl, (C₂-C₈) alkenyl, (C₂-C₈) alkynyl, aryl, and heteroaryl, each of which may be optionally substituted. In certain embodiments, the population of monomeric residues can be derived from polymerization of (C₂-C₈) alkyl ethacrylate, a (C₂-C₈) alkyl methacrylate, or a (C₂-C₈) alkyl acrylate (each of which may be optionally substituted).

In a preferred embodiment, the hydrophobic block comprises anionic repeat units having a population of pendant anions that vary in number in a pH dependant manner. In general, the population of pendant anions is greater at pH 7.4 than at pH 5. In general, it is preferred that at least 90% of the population of these pH-sensitive anionic repeat units are non-charged at about pH 5. For example, in one such preferred embodiment, at least 99% of the population of these pH-sensitive anionic repeat units are non-charged at about pH 5. By way of further example, in one such embodiment the anionic repeat units comprise pendant groups that are in the form of carboxyl anions at pH 7.4 and carboxylic acids at pH 5.

It is also preferred that the hydrophobic block comprise a substantial number of anionic repeat units having a population of pendant anions that vary in number in a pH dependant manner. For example, in one embodiment, the hydrophobic block comprises at least 10 such residues. In another embodiment, it comprises as least 20 such residues. In another embodiment, it comprises at least 50 such residues. In another embodiment, it comprises at least 100 such residues. In such embodiments, the hydrophobic block will typically comprise about 10 to about 500 anionic residues.

In some preferred embodiments, the hydrophobic block comprises a population of anionic hydrophobic monomeric residues, i.e., monomeric residues comprising both hydrophobic species (e.g., a C₂-C₈ alkyl substituent) and species charged or chargeable to an anion. In each of such aforementioned embodiments, the hydrophobic block can be considered hydrophobic in the aggregate.

In general, therefore, the hydrophobic block may contain anionic repeat units, cationic repeat units, zwitterionic repeat units, a combination of two or more charged repeat units (e.g., anionic and cationic repeat units, anionic and zwitterionic repeat units, cationic and zwitterionic repeat units, or anionic, cationic and zwitterionic repeat units), substantially non-charged repeat units, or a combination thereof, provided that its overall character is hydrophobic. Stated differently, the hydrophobic block may contain any of a wide range of repeat units, hydrophobic or even hydrophilic, provided that the sum of the contributions of the repeat units comprised by the hydrophobic block provide a block having an overall hydrophobic character. When the repeat units contain ionizable groups, the contribution of an individual repeat unit to the overall hydrophilicity of the block of which it is a constituent may vary as a function of its pKa relative to the pH of the environment in which it is found. For example, propyl acrylic acid repeat units, —CH₂C(CH₂CH₂CH₃)(COOH)—, are predominantly ionized at pH 7 but not at pH 5 and thus, the hydrophobic contribution of propyl acrylic acid repeat units to a block is significantly greater at pH 5 than at pH 7. In general, therefore, it is preferred that the sum of the contributions of the repeat units constituting the hydrophobic block be such that the overall character of the block is hydrophobic at pH's that are less than physiological pH. For example, in one embodiment, the sum of the contributions is such that the overall character of the block is hydrophobic at a pH of about 5.0. By way of further example, in one embodiment, the sum of the contributions is such that the overall character of the block is hydrophobic at a pH of about 5.5. By way of further example, in one embodiment, the sum of the contributions is such that the overall character of the block is hydrophobic at a pH of about 6.0. By way of further example, in one embodiment, the sum of the contributions is such that the overall character of the block is hydrophobic at a pH of about 6.8. By way of further example, in one embodiment, the sum of the contributions of the repeat units is such that the overall character of the hydrophobic block is hydrophobic at a pH within the range of about 6.2 to 6.8.

In certain embodiments, a hydrophobic block described herein comprises monomeric residues resulting from the polymerization or copolymerization of a monomer comprising a hydrophobic species. Monomers comprising a hydrophobic species include, by way of non-limiting example, optionally substituted, (C₂-C₈)alkyl-ethacrylate, a (C₂-C₈)alkyl-methacrylate, a (C₂-C₈)alkyl-acrylate, styrene, (C₂-C₈)alkyl-vinyl, or the like. In certain embodiments, monomers comprising a hydrophobic species include, by way of non-limiting example, monomers of Formula VI:

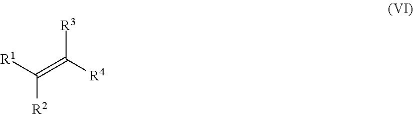

wherein:

R¹ and R² are each independently hydrogen, halogen, optionally substituted C₁-C₆ alkyl, optionally substituted alkenyl, optionally substituted alkynyl, optionally substituted cycloalkyl, optionally substituted aryl, or optionally substituted heteroaryl;

R³ is hydrogen, halogen, optionally substituted C₁-C₆ alkyl, or —C(═O)R⁵;

R⁴ is hydrogen, halogen, optionally substituted C₁-C₆ alkyl;

R⁵ is optionally substituted C₁-C₆ alkyl, —SR⁶, —OR⁶, or —NR⁷R⁸;

R⁶ is hydrogen, optionally substituted C₁-C₆ alkyl, optionally substituted alkenyl, optionally substituted alkynyl, optionally substituted cycloalkyl, optionally substituted aryl, or optionally substituted heteroaryl;

R⁷ and R⁸ are each independently hydrogen, optionally substituted alkyl, optionally substituted heteroalkyl, optionally substituted cycloalkyl, optionally substituted aryl; or R⁷ and R⁸ together with the nitrogen to which they are attached form an optionally substituted heterocycle.

In certain embodiments, in addition to a hydrophobic species, a hydrophobic block described herein further comprises an anionic species. In specific embodiments, the anionic species is anionic at about neutral pH. In specific embodiments, the hydrophobic block comprises a first monomer residue comprising a hydrophobic species and a second monomer residue comprising an anionic species.

In certain embodiments, a hydrophobic block described herein comprises a polymer block comprising a plurality of hydrophobic monomeric residues and a plurality of anionic monomeric residues. In some embodiments, a hydrophobic block described herein comprises a polymer block comprising a plurality of chargeable residues. In some embodiments, monomeric residues that are anionic, (1) are anionic at about neutral pH, a pH greater than about 7.2, or any pH greater than about 7.4, and (2) are non-charged at a pH of less than about 6, less than about 5.8, less than about 5.7, less than about 5.6, less than about 5.5, less than about 5.4, less than about 5.2, less than about 5.0, or less than about 4.5. In some embodiments, the monomeric residues have a pKa anywhere between about 4.5 and about 8.0, anywhere between about 5.5 and about 7.5, or anywhere between about 6.0 and about 7.0. In certain embodiments, monomers that when polymerized provide the anionic monomeric residues have a pKa anywhere between about 4.5 and about 8.0, anywhere between about 5.5 and about 7.5, or anywhere between about 6.0 and about 7.0.

In specific embodiments, anionic species which may be found on anionic monomeric residues described herein include, by way of non-limiting example, carboxylic acid, sulfonamide, boronic acid, sulfonic acid, sulfinic acid, sulfuric acid, phosphoric acid, phosphinic acid, and phosphorous acid groups, or the conjugate bases or anions thereof. In some embodiments, the anionic monomeric residue is a residue of (C₁-C₈)alkylacrylic acid, or acrylic acid. In certain embodiments monomers such as maleic-anhydride, (Scott M. Henry, Mohamed E. H. El-Sayed, Christopher M. Pirie, Allan S. Hoffman, and Patrick S. Stayton, pH-Responsive Poly(styrene-alt-maleic anhydride) Alkylamide Copolymers for Intracellular Drug Delivery. Biomacromolecules 2006, 7, 2407-2414) are used for introduction of anionic species by post-polymerization hydrolysis of the maleic anhydride monomeric units.

In certain embodiments, in addition to a hydrophobic species, a hydrophobic block described herein further comprises a cationic species. In some embodiments, in addition to a hydrophobic species and an anionic species, a hydrophobic block described herein further comprises a cationic species. In certain embodiments, in addition to a hydrophobic monomeric residue, a hydrophobic block described herein further comprises a cationic monomeric residue. In some embodiments, in addition to a hydrophobic monomeric residue and an anionic monomeric residue, a hydrophobic block described herein further comprises a cationic monomeric residue. In some embodiments, species and/or monomeric residues that are cationic are cationic at about neutral pH.

In some embodiments, a cationic monomeric residues described herein has a pKa ranging anywhere between about 6.0 and about 10.0, typically between about 6.2 and about 9.5, and in some embodiments between about 6.5 and about 8.5. Upon incorporation of the monomer into the polymer block, the pKa of the residue tends to decrease relative to the unpolymerized monomer; in general, therefore, the pKa of the incorporated repeat units will be between about 6.0 and 10.0, typically between about 6.2 and 9.0, and in some embodiments, between about 6.5 and 8.0.

In certain embodiments, the hydrophobic block comprises a monomeric species comprising an acyclic amine (e.g., an amine, an alkyl amine, a dialkyl amine, or the like), an acyclic imine (e.g., an imine, an alkyl imine, or the like), a cyclic amine (e.g., piperidine), a nitrogen containing heterocycle (e.g., pyridine or quinoline), or the like. In specific embodiments, a cationic species utilized herein includes a protonated acyclic amine (e.g., an amine, an alkyl amine, a dialkyl amine, or the like), an acyclic imine (e.g., an imine, an alkyl imine, or the like), a cyclic amine (e.g., piperidine), a nitrogen containing heterocycle (e.g., pyridine or quinoline), or the like.

Non-limiting examples of acyclic amines include methylamine, dimethylamine, ethylamine, diethylamine, propylamine, isopropylamine, diisopropylamine, diisopropylethylamine, n-butylamine, sec-butylamine, tert-butylamine, pentylamine, neo-pentylamine, iso-pentylamine, hexanamine or the like. Non-limiting examples of acyclic imines include methylimine, ethylimine, propylimine, isopropylimine, n-butylimine, sec-butylimine, pentylimine, neo-pentylimine, iso-pentylimine, hexylimine or the like. Non-limiting examples of cyclic amines include cyclopropylamine, cyclobutylamine, cyclopentylamine, cyclohexylamine, cycloheptylamine, piperidine, pyrazine, pyrrolidine, homopiperidine, azabicylcoheptane, diazabicycloundecane, or the like. Non-limiting examples of cyclic imines include cyclopropylimine, cyclobutylimine, cyclopentylimine, cyclohexylimine, cycloheptylimine, or the like. Non-limiting examples of nitrogen containing heteroaryls include imidazolyl, pyrrolyl, pyridyl, indolyl, or the like.

In some embodiments, a hydrophobic block of a polymer described herein comprises a plurality of monomeric residues of optionally substituted, amino(C₁-C₆)alkyl-ethacrylate, amino(C₁-C₆)alkyl-methacrylate, amino(C₁-C₆)alkyl-acrylate, (N—(C₁-C₆)alkyl-amino(C₁-C₆)alkyl-ethacrylate, N—(C₁-C₆)alkyl-amino(C₁-C₆)alkyl-methacrylate, N—(C₁-C₆)alkyl-amino(C₁-C₆)alkyl-acrylate, (N,N-di(C₁-C₆)alkyl-amino(C₁-C₆)alkyl-ethacrylate, N,N-di(C₁-C₆)alkyl-amino(C₁-C₆)alkyl-methacrylate, N,N-di(C₁-C₆)alkyl-amino(C₁-C₆)alkyl-acrylate, or a combination thereof. In specific embodiments, such monomeric residues constitute a cationic monomeric residue at neutral pH as described herein.

In certain embodiments, a hydrophobic block described herein comprises a plurality of anionic monomeric residues, a plurality of cationic monomeric residues, and a plurality of hydrophobic monomeric residues.

In certain specific embodiments, at about neutral pH (e.g., at about pH 7.4), the hydrophobic block has a substantially neutral overall charge. In still more specific embodiments, a substantially neutral overall charge of the hydrophobic block means that at least 60%, at least 70%, at least 80%, at least 90%, at least 95%, or at least 98% of the charge of either of the cationic monomeric residues or the anionic monomeric residues are neutralized by the charge of the of the other of the cationic monomeric residues or the anionic monomeric residues. In other words, in various embodiments, the ratio of the number of monomeric units in the hydrophobic block that are cationic at about neutral pH to the number of monomeric units in the hydrophobic block that are anionic at about neutral pH is between about 3:5 and about 5:3, about 7:10 and about 10:7, about 4:5 and about 5:4, about 9:10 and about 10:9, about 95:100 and about 100:95, or about 98:100 and about 100:98. Determination of charge ratios can be achieved in any suitable manner, e.g., by calculating the amount of charged species using the pKa and/or pKb values thereof.

In some embodiments, at about neutral pH (e.g., at about pH 7.4), the hydrophobic block comprises anionic species and cationic species in a ratio of about 10:1 to about 1:10, about 5:1 to about 1:5, about 4:1 to about 1:4, about 1:0 to about 1:4 (anionic species:cationic species). In a preferred embodiment, at about neutral pH (e.g., at about pH 7.4), the hydrophobic block comprises anionic species and cationic species in a ratio of about 1:1 (anionic species:cationic species). In some embodiments, at about neutral pH (e.g., at about pH 7.4), the hydrophobic block comprises anionic monomeric residues and cationic monomeric residues in a ratio of about 1:0 to about 1:4 (anionic monomeric residues:cationic monomeric residues). In a preferred embodiment, at about neutral pH (e.g., at about pH 7.4), the hydrophobic block comprises anionic monomeric residues and cationic monomeric residues in a ratio of about 1:1 (anionic monomeric residues:cationic monomeric residues).

In certain embodiments, at about neutral pH (e.g., at about pH 7.4), the hydrophobic block comprises hydrophobic monomeric residues, cationic monomeric residues, and anionic monomeric residues. In specific embodiments, the ratio of hydrophobic monomeric residues to charged monomeric residues (cationic monomeric residues plus anionic monomeric residues), is about 1:5 to about 5:1, or about 1:3 to about 3:1, or about 1:2 to about 3:1, or about 1:1, or about 1:2, or about 2:1.

In certain embodiments, a hydrophobic block, as described herein comprises a plurality of first monomeric residues derived from a first polymerizable monomer having a protonatable anionic species and a hydrophobic species, and optionally a plurality of second monomeric residues derived from a second polymerizable monomer having a deprotonatable cation species.

In one preferred embodiment, the hydrophobic block comprises repeat units corresponding to Formula 1

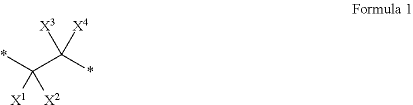

wherein * designates the point of attachment of the repeat unit of Formula 1 to other repeat units; each X¹ and X² is independently selected from the group consisting of hydrogen, hydrocarbyl, substituted hydrocarbyl, heterocyclo, and substituted carbonyl, provided, however, X¹ and X² are not, in the same repeat unit, selected from the group consisting of aryl, heteroaryl, heterosubstituted carbonyl, and combinations thereof; each X³ is independently hydrogen, alkyl or substituted alkyl, and each X⁴ is independently heterosubstituted carbonyl, aryl, or heteroaryl. For example, in one such embodiment the hydrophobic block comprises repeat units corresponding to Formula 1 and X¹ and X² are each hydrogen. In another such example, the hydrophobic block comprises repeat units corresponding to Formula 1, X¹ and X² are each hydrogen and X³ is hydrogen or alkyl. In a further example, the hydrophobic block comprises repeat units corresponding to Formula 1, X¹ and X² are each hydrogen, X³ is hydrogen or alkyl, and each X⁴ is independently heterosubstituted carbonyl. In a further example, the hydrophilic block comprises repeat units corresponding to Formula 1, X⁴ is —C(O)OX⁴⁰, —C(O)SX⁴⁰, or —C(O)NX⁴⁰X⁴¹, and X⁴⁰ and X⁴¹ are independently hydrogen, hydrocarbyl, substituted hydrocarbyl, heterohydrocarbyl, substituted heterohydrocarbyl, or heterocyclo. In a further example, the hydrophobic block comprises repeat units corresponding to Formula 1, X⁴ is —C(O)OX⁴⁵, —C(O)SX⁴⁵, or —C(O)NX⁴¹X⁴⁵, and X⁴¹ and X⁴⁵ are independently hydrogen, hydrocarbyl, substituted hydrocarbyl, heterohydrocarbyl, or heterocyclo. In a further example, the hydrophobic block comprises repeat units corresponding to Formula 1, X⁴ is —C(O)OX⁴⁵ or —C(O)NX⁴¹X⁴⁵, and X⁴¹ and X⁴⁵ are independently hydrocarbyl, heterohydrocarbyl, or heterocyclo. In a further example, the hydrophobic block comprises repeat units corresponding to Formula 1, X¹ and X² are each hydrogen, X³ is hydrogen or alkyl, X⁴ is —C(O)OX⁴⁵, and X⁴⁵ is hydrogen, hydrocarbyl, substituted hydrocarbyl, heterohydrocarbyl, substituted heterohydrocarbyl, or heterocyclo. In a further example, the hydrophobic block comprises repeat units corresponding to Formula 1, X¹ and X² are each hydrogen, X³ is hydrogen or alkyl, X⁴ is —C(O)OX⁴⁵, X⁴⁵ is a disulfide substituted alkyl moiety (for example, pyridyl disulfide substituted ethyl). In a further example, the hydrophobic block comprises repeat units corresponding to Formula 1, X¹ and X² are each hydrogen, X³ is hydrogen or alkyl, X⁴ is —C(O)NX⁴¹X⁴⁵, X⁴¹ is hydrogen, and X⁴⁵ is alkyl or substituted alkyl. In yet a further example, the hydrophobic block comprises repeat units corresponding to Formula 1, where X⁴ is —C(O)OX⁴⁶ or —C(O)NX⁴⁶X⁴¹, X⁴⁶ is substituted hydrocarbyl, substituted heterohydrocarbyl, or heterocyclo, X⁴⁶ comprises the polynucleotide, and the polynucleotide is covalently bound to the composition through X⁴⁶.

In one alternative embodiment, the hydrophobic block is a random copolymer comprising at least two compositionally distinct repeat units, each of which corresponds to Formula 1. For example, the hydrophobic block may be a random copolymer comprising (i) a first repeat unit corresponding to Formula 1 in which X⁴ is —C(O)OX⁴⁵ and (ii) a second repeat unit corresponding to Formula 1 in which X⁴ is —C(O)NX⁴¹X⁴⁵. Advantageously, when the hydrophobic block is a random copolymer comprising at least two compositionally distinct repeat units, one of the repeat units may provide a functional group for attaching a therapeutic agent such as a nucleic acid. Thus, for example, the hydrophobic block may comprise (i) a first repeat unit corresponding to Formula 1 wherein X⁴ is —C(O)OX⁴⁵, —C(O)SX⁴⁵, or —C(O)NX⁴¹X⁴⁵ and a therapeutic agent such as a nucleic acid is attached to the hydrophobic block via X⁴⁵ or X⁴⁵ comprises a functional group for attaching the therapeutic agent, and (ii) a compositionally distinct repeat unit corresponding to Formula 1. For example, X⁴⁵ may be an alkyl group substituted by N-hydroxysuccinimide (NHS)ester, HOBt (1-hydroxybenzotriazole) ester, p-nitrophenyl ester, tetrafluorophenyl ester, pentafluorophenyl ester, pyridyl disulfide group, maleimide, aldehyde, ketone, anhydride, thiol, amine, hydroxyl, alkyl halide, or the like.

In one preferred embodiment, the hydrophobic block comprises repeat units corresponding to Formula 1A

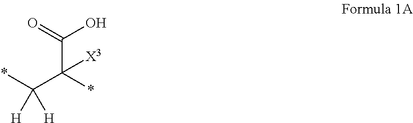

wherein * designates the point of attachment of the repeat unit of Formula 1A to other repeat units; and X³ is alkyl. Exemplary alkyls include methyl, ethyl, propyl and butyl. Typically, X³ is ethyl or propyl. In a preferred embodiment, X³ is propyl. It is also preferred that the hydrophobic block comprise a substantial number of anionic repeat units corresponding to Formula 1A. For example, in one embodiment, the hydrophobic block comprises at least 10 such residues. In another embodiment, it comprises as least 20 such residues. In another embodiment, it comprises at least 50 such residues. In another embodiment, it comprises at least 100 such residues. In such embodiments, the hydrophobic block will typically comprise about 10 to about 500 anionic residues.

In one preferred embodiment, the hydrophobic block comprises repeat units corresponding to Formula 1E

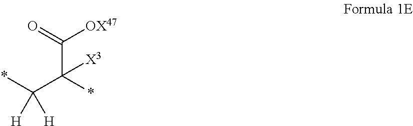

wherein * designates the point of attachment of the repeat unit of Formula 1E to other repeat units; and X³ and X⁴⁷ are independently alkyl. Exemplary alkyls include methyl, ethyl, propyl and butyl. Typically, X³ is methyl, ethyl or propyl and X⁴⁷ is independently methyl, ethyl, propyl or butyl. In one preferred embodiment, X³ is methyl and X⁴⁷ is butyl.

In one preferred embodiment, the hydrophobic block comprises repeat units corresponding to Formula 1C

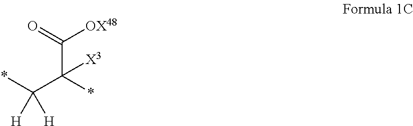

wherein * designates the point of attachment of the repeat unit of Formula 1C to other repeat units; X³ is alkyl, and X⁴⁸ is amino-substituted alkyl. Exemplary alkyls include methyl, ethyl, propyl and butyl. Typically, X³ is ethyl or propyl and X⁴⁸ is N,N-dialkylaminoalkyl, e.g., N,N-dimethylaminoethyl.

In one preferred embodiment, the hydrophobic block comprises repeat units corresponding to Formula 1-CON

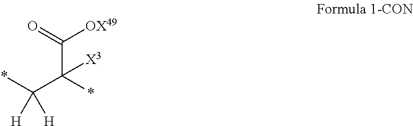

wherein * designates the point of attachment of the repeat unit of Formula 1-CON to other repeat units; X³ is hydrogen or alkyl, and X⁴⁹ is substituted hydrocarbyl, heterohydrocarbyl, or heterocyclo. In one embodiment, X⁴⁹ comprises a conjugatable group. For example, X⁴⁹ may be an alkyl group substituted by N-hydroxysuccinimide (NHS)ester, HOBt (1-hydroxybenzotriazole) ester, p-nitrophenyl ester, tetrafluorophenyl ester, pentafluorophenyl ester, pyridyl disulfide group, maleimide, aldehyde, ketone, anhydride, thiol, amine, hydroxyl, alkyl halide, or other conjugatable group. In an alternative embodiment, X⁴⁹ may be a conjugatable group selected from succinimidyl, benzotriazyl, p-nitrophenyl, tetrafluorophenyl, or pentafluorophenyl.

In one preferred embodiment, the hydrophobic block is a random copolymer comprising (i) repeat units corresponding to Formula 1A and Formula 1E, (ii) repeat units corresponding to Formula 1A and 1C, (iii) repeat units corresponding to Formula 1A, 1C and 1E, (iv) repeat units corresponding to Formula 1-CON and Formula 1A, (v) repeat units corresponding to Formula 1-CON, Formula 1A and Formula 1C, (vi) repeat units corresponding to Formula 1-CON, Formula 1A and Formula 1E, and (vii) repeat units corresponding to Formula 1-CON, Formula 1A and 1C, and (viii) repeat units corresponding to Formula 1-CON, Formula 1A, 1C and 1E. When the hydrophobic block comprises repeat units corresponding to Formula 1-CON, they will typically not constitute more than about 20% of the repeat units in the hydrophobic block. More typically, repeat units corresponding to Formula 1-CON, will not constitute more than about 15% of the repeat units in the hydrophobic block. In some embodiments, repeat units corresponding to Formula 1-CON, will constitute about 5% to about 10% of the repeat units in the hydrophobic block. In general, when the hydrophobic block is a random copolymer comprising repeat units corresponding to Formula 1A and Formula 1E (with or without repeat units corresponding to Formula 1C), the ratio of the number of repeat units corresponding to Formula 1A to the number of repeat units corresponding to Formula 1E in the third block is between about 20:1 and 1:4, respectively. For example, it is generally preferred that the ratio of the number of repeat units corresponding to Formula 1A to the number of repeat units corresponding to Formula 1E in the third block be between about 3:1 and 1:3, respectively. Additionally, it is generally preferred that the number of repeat units corresponding to Formula 1A exceed the number of repeat units corresponding to Formula 1C.

In certain embodiments, the hydrophobic block comprises monomeric residues derived from polymerization of conjugatable monomers having Formula VIII:

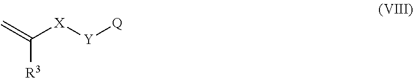

wherein:

X is selected from a group consisting of a covalent bond, C(O), a divalent C₅-C₁₀ aryl, and a divalent C₂-C₁₀ heteroaryl,

Y is a covalent bond or a linking group selected from optionally substituted divalent C₁-C₂₀ alkyl, optionally substituted divalent C₁-C₂₀ heteroalkyl, optionally substituted divalent C₁-C₂₀ alkenyl, optionally divalent substituted C₁-C₂₀ alkynyl, optionally substituted divalent C₁-C₂₀ cycloalkyl, optionally substituted divalent C₁-C₂₀ cycloheteroalkyl optionally substituted divalent C₅-C₁₀ aryl, or optionally substituted divalent C₃-C₁₀ heteroaryl,

R³ is selected from the group consisting of hydrogen and optionally substituted C₁-C₄ alkyl,

Q is the conjugatable group such as but not limited to N-hydroxysuccinimide (NHS)ester, HOBt (1-hydroxybenzotriazole) ester, p-nitrophenyl ester, tetrafluorophenyl ester, pentafluorophenyl ester, pyridyl disulfide group, maleimide, aldehyde, ketone, anhydride, thiol, amine, hydroxyl, alkyl halide, or the like.

In certain embodiments, the hydrophobic block corresponds to the formula:

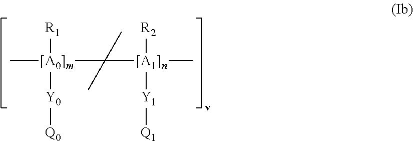

wherein:

A₀, and A₁ are selected from the group consisting of —C—C—, —C—, —C(O)(C)ₐC(O)O—, —O(C)ₐC(O)— and —O(C)_{b}O—;

a is 1-4;

b is 2-4;

Y₀, and Y₁ are independently selected from the group consisting of a covalent bond, (1C-10C)alkyl-, —C(O)O(2C-10C) alkyl-, —OC(O)(1C-10C) alkyl-, —O(2C-10C)alkyl- and —S(2C-10C)alkyl- —C(O)NR₆(2C-10C) alkyl-;

tetravalent carbon atoms of A₀-A₁ that are not fully substituted with R₁-R₂ and Y₀-Y₁ are completed with an appropriate number of hydrogen atoms;

each R₁, R₂, and R₆ are independently selected from the group consisting of hydrogen, —CN, alkyl, alkynyl, heteroalkyl, cycloalkyl, heterocycloalkyl, aryl and heteroaryl, any of which may be optionally substituted with one or more fluorine atoms;

Q₀ is a residue selected from the group consisting of residues which are hydrophilic at physiologic pH and are at least partially positively charged at physiologic pH (e.g., amino, alkylamino, ammonium, alkylammonium, guanidine, imidazolyl, pyridyl, or the like); conjugatable or functionizable residues (e.g. residues that comprise a reactive group, e.g., azide, alkyne, succinimide ester, tetrafluorophenyl ester, pentafluorophenyl ester, p-nitrophenyl ester, pyridyl disulfide, or the like); or hydrogen;

Q₁ is a residue which is hydrophilic at physiologic pH, and is at least partially positively charged at physiologic pH (e.g., amino, alkylamino, ammonium, alkylammonium, guanidine, imidazolyl, pyridyl, or the like); at least partially negatively charged at physiologic pH but undergoes protonation at lower pH (e.g., carboxyl, sulfonamide, boronate, phosphonate, phosphate, or the like); substantially neutral at physiologic pH (e.g., hydroxy, polyoxylated alkyl, polyethylene glycol, polypropylene glycol, thiol, or the like); or at least partially zwitterionic at physiologic pH (e.g., a monomeric residue comprising a phosphate group and an ammonium group at physiologic pH);

m is 0 to less than 1.0 (e.g., 0 to about 0.49);

n is greater than 0 to 1.0 (e.g., about 0.51 to about 1.0); wherein

m+n=1; and

v is from about 1 to about 25 kDa.

In certain embodiments, the conjugatable or functionizable residue of Q₀ is conjugated to at least one amino acid or at least one nucleotide.

In one preferred embodiment, the hydrophobic block comprises repeat units corresponding to Formula 1A, 1E, 1C and 1-CON wherein each X³ is independently hydrogen or alkyl, X⁴⁷ is alkyl, X⁴⁸ is amino-substituted alkyl, and X⁴⁹ comprises a conjugatable group. For example, X⁴⁹ may be an alkyl group substituted by N-hydroxysuccinimide (NHS)ester, HOBt (1-hydroxybenzotriazole) ester, p-nitrophenyl ester, tetrafluorophenyl ester, pentafluorophenyl ester, pyridyl disulfide group, maleimide, aldehyde, ketone, anhydride, thiol, amine, hydroxyl, alkyl halide, or other conjugatable group. Exemplary alkyls include methyl, ethyl, propyl and butyl. Typically, X³ is methyl, ethyl or propyl, X⁴⁸ is N,N-dialkylaminoalkyl, and X⁴⁹ is alkyl substituted by pyridyl disulfide. Thus, for example, in one embodiment, the hydrophobic block comprises repeat units corresponding to Formulae 1A, 1C, 1E, 1-CON and the relative mole ratio of these repeat units is about 25:25:40:10, respectively. In another exemplary embodiment, the hydrophobic block comprises repeat units corresponding to Formulae 1A, 1C, 1E, 1-CON and the relative mole ratio of these repeat units is about 25:25:45:5, respectively. In another exemplary embodiment, the hydrophobic block comprises repeat units corresponding to Formulae 1A, 1C, 1E, 1-CON and the relative mole ratio of these repeat units is about 20:20:50:10, respectively. In one preferred embodiment, the hydrophobic block comprises, as repeat units, the residues of 2-propylacrylic acid, N,N-dimethylaminoethyl methacrylate, butyl methacrylate, and pyridyldisulfide methacrylate ester. In certain preferred embodiments, the hydrophobic block comprises, as repeat units, the residues of 2-propylacrylic acid (a Formula 1A constituent), N,N-dimethylaminoethyl methacrylate (a Formula 1C constituent), butyl methacrylate (a Formula 1E constituent), and pyridyldisulfide methacrylate ester (a Formula 1-CON constituent) in any of the ratios disclosed herein for Formulae 1A, 1C, 1E, and 1-CON, respectively.

In general, the hydrophobic block comprises a plurality of repeat units, i.e., at least two. In certain embodiments, a hydrophobic block of a polymer described herein has a number average molecular weight of about 1,000 Dalton to about 200,000 Dalton, about 1,000 Dalton to about 100,000 Dalton, about 1,000 Dalton to about 100,000 Dalton, about 5,000 Dalton to about 50,000 Dalton, about 10,000 Dalton to about 50,000 Dalton, about 15,000 Dalton to about 35,000 Dalton, or about 20,000 Dalton to about 30,000 Dalton.

Hydrophilic Block

As previously noted, the polymer optionally comprises at least one hydrophilic block. In certain embodiments, therefore, the polymer will be a diblock polymer comprising a hydrophilic block and a hydrophobic block. In other embodiments, the polymer will be a multiblock polymer comprising two or more compositionally distinct hydrophilic blocks. It should be understood, therefore, that when the polymer comprises two or more hydrophilic blocks, each of the hydrophilic blocks may be independently selected from hydrophilic blocks described herein.

In some embodiments, the polymer comprises at least one hydrophilic block comprising a plurality of charged species (i.e., is polycationic, polyanionic, or both), and/or is associated with a molecule that comprises a plurality of charged species (e.g., a polynucleotide or polypeptide). In specific embodiments, the polymer comprises at least one hydrophilic block comprising a population of species that are charged at about neutral pH (i.e., is polycationic, polyanionic, or both at about neutral pH). In certain embodiments, at least one of the hydrophilic blocks is a shielding block. In specific embodiments, the shielding block is non-charged, e.g., at about neutral pH.

Hydrophilic block(s) may be used to contribute a range of properties or functions to the copolymer of the present invention. For example, the number and composition of the constituent units of the hydrophilic block(s) may be selected to impart the desired degree of water solubility/dispersability to the copolymer. Alternatively, or additionally, in those embodiments in which the polymers of the present invention are incorporated into micelles, the number and composition of the constituent units of the hydrophilic block(s) may be selected to provide micelles having a desired size, critical micelle concentration or other property. Independent of micelle formation, the number and composition of the constituent units of the hydrophilic block(s) may be selected to target the polymer to a cellular or other biological target. Alternatively, or additionally, the number and composition of the constituent units of the hydrophilic block(s) may be selected to shield a therapeutic agent that is associated with the copolymer.

Depending upon the desired properties and functionality, the hydrophilic block(s) may be a homopolymer block or a copolymer block. In those embodiments in which it is a copolymer block, it is preferably a random copolymer block. For example, the hydrophilic block(s) may be copolymer block(s) comprising two or more compositionally distinct monomeric residues. By way of further example, the hydrophilic block(s) may be copolymer block(s) comprising charged repeat units (i.e., cationic repeat units, anionic repeat units, zwitterionic repeat units or a combination thereof), non-charged repeat units, or a combination thereof. In one specific embodiment, the hydrophilic block(s) comprise(s) charged repeat units. In another specific embodiment, the hydrophilic block(s) comprise(s) cationic repeat units. In yet another embodiment, the hydrophilic block comprise(s) cationic repeat units and non-charged repeat units. In yet another embodiment, the hydrophilic block(s) comprise(s) zwitterionic repeat units and non-charged repeat units. In yet another embodiment, the hydrophilic block(s) comprise(s) exclusively non-charged repeat units and the block is non-charged at neutral pH. Furthermore, in those embodiments in which the hydrophilic block(s) comprise(s) cationic repeat units, the hydrophilic block(s) may comprise a combination of two or more compositionally distinct cationic repeat units; in those embodiments in which the hydrophilic block(s) comprise(s) anionic repeat units, the hydrophilic block may comprise a combination of two or more compositionally distinct anionic repeat units; in those embodiments in which the hydrophilic block(s) comprise(s) zwitterionic repeat units, the hydrophilic block may comprise a combination of two or more compositionally distinct zwitterionic repeat units; and in those embodiments in which the hydrophilic block(s) comprise(s) non-charged repeat units, the hydrophilic block(s) may comprise a combination of two or more compositionally distinct non-charged repeat units.

In general, the hydrophilic block(s) may contain anionic repeat units, cationic repeat units, zwitterionic repeat units, a combination of two or more charged repeat units (e.g., anionic and cationic repeat units, anionic and zwitterionic repeat units, cationic and zwitterionic repeat units, or anionic, cationic and zwitterionic repeat units), substantially non-charged repeat units, or a combination thereof, provided that its overall character is hydrophilic. Stated differently, the hydrophilic block(s) may contain any of a wide range of repeat units, hydrophilic or even hydrophobic, provided that the sum of the contributions of the repeat units comprised by the first hydrophilic block provide a block having an overall hydrophilic character. When the repeat units contain ionizable groups, the contribution of an individual repeat unit to the overall hydrophilicity of the block of which it is a constituent may vary as a function of its pKa relative to the pH of the environment in which it is found. For example, propyl acrylic acid repeat units, —CH₂C(CH₂CH₂CH₃)(COOH)—, are predominantly ionized at pH 7 but not at pH 5 and thus, the hydrophobic contribution of propyl acrylic acid repeat units to a block is significantly greater at pH 5 than at pH 7. In general, therefore, when the first hydrophilic block comprises ionizable cationic repeat units or ionizable anionic repeat units, it is preferred that the sum of the contributions of the repeat units constituting the first hydrophilic block be such that the overall character of the block is hydrophilic at physiological pH. For example, in one embodiment, it is preferred that the hydrophilic block(s) be hydrophilic over the range of pH from pH 5.0 to pH 7.5.

In some embodiments, the hydrophilic block is a substantially non-charged block. In specific embodiments, the hydrophilic block is substantially non-charged and shields a charged second block and/or a charged therapeutic agent associated with the hydrophobic block. For example, the hydrophilic block may be a polysaccharide block.

In some other embodiments, the hydrophilic block is a polyzwitterionic (i.e., comprising a plurality of both cationic and anionic species or monomeric residues at about neutral pH), polyanionic block (i.e., comprising a plurality of anionic monomeric residues at about neutral pH), or polycationic block (i.e., comprising a plurality of cationic monomeric residues at about neutral pH). In either of these embodiments, the hydrophilic block may comprise at least one conjugatable or functionizable monomeric residue that may be used, for example, to link a targeting group, a water-solubilizing group, or other functional group to the polymer block, post-polymerization.

In one preferred embodiment, the hydrophilic block is a hydrophilic polymer block suitable for (capable of/effective for) steric shielding of the polynucleotide. In this embodiment, the hydrophilic block comprises a plurality of monomeric residues having a shielding species, and the shielding species is effective for steric shielding of a polynucleotide associated with the polymer or a micelle comprising the polymer. For example, the shielding species may be effective for enhancing the stability of the polynucleotide against enzymatic digestion in plasma. Additionally, or alternatively, the shielding species may be effective for reducing toxicity of a polymer or micelle described (e.g., by shielding a charged second hydrophilic block and reducing the effective surface charge of a polymer or micelle). Additionally, or alternatively, the shielding species is effective for maintaining desirable surface properties of a micelle comprising the polymer. Additionally, or alternatively, the shielding species may be effective for increasing the circulation time in or decreasing its clearance rate by the kidneys.

Depending upon the desired properties, the shielding species of a polymer or micelle may be selected from a range of moieties. For example, the shielding species may be an alcohol, a phenol, a thiol, a polyoxylated alkyl (e.g., polyethylene glycol, polypropylene glycol, or the like), an ether, a thio-ether, or the like. In some embodiments, the hydrophilic block comprises a plurality of monomeric residues having a pendant group comprising a shielding oligomer (e.g., polyethylene glycol, polypropylene glycol, or the like). In specific embodiments, the shielding species of a polymer or micelle described herein is a polyoxylated alkyl with the structure of Formula II:

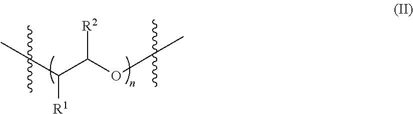

wherein each R¹ and R² are each independently selected from the group consisting of hydrogen, halogen, and optionally substituted C₁-C₃ alkyl, and n is an integer. In a preferred embodiment, the number of repeat units, n, will provide a shielding species having a molecular weight of about 40 to about 2000 daltons. In specific embodiments, n will be about 2-20; for example, n may be about 3-10 and, depending upon the polymer, about 4-5, about 8-9, or the like. In some embodiments, the shielding species corresponds to Formula II and has a molecular weight of about 40 to about 2000 dalton, or about 100 to about 2000 dalton.

In some embodiments, the hydrophilic block has a structure corresponding to Formula Ia-1:

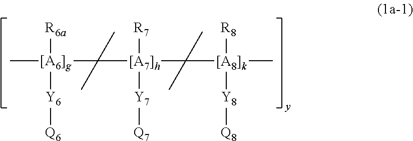

wherein:

each of A₆, A₇, and A₈ are independently selected from the group consisting of —C—C—, —C—, —C(O)(C)ₐC(O)O—, —O(C)ₐC(O)— and —O(C)_{b}O—;

a is 1-4;

b is 2-4;

each of Y₆, Y₇, and Y₈ are independently selected from the group consisting of a covalent bond, (1C-10C)alkyl-, (3C-6C)cycloalkyl, O-(1C-10C)alkyl, —C(O)O(2C-10C) alkyl-, —OC(O)(1C-10C) alkyl-, —O(2C-10C)alkyl-, —S(2C-10C)alkyl-, —C(O)NR₆(2C-10C) alkyl-, and aryl, any of which is optionally substituted;

tetravalent carbon atoms of A₆-A₈ that are not fully substituted with R₆ₐ, R₇, R₈ and Y₆-Y₈ are completed with an appropriate number of hydrogen atoms;

each R₆ₐ, R₇, and R₈ is independently selected from the group consisting of hydrogen, —CN, alkyl, alkynyl, heteroalkyl, cycloalkyl, heterocycloalkyl, aryl and heteroaryl, any of which may be optionally substituted with one or more fluorine atoms;

Q₆ is a residue selected from the group consisting of residues which are hydrophilic at physiologic pH and are substantially non-charged at about neutral pH (e.g., hydroxy, polyoxylated alkyl, polyethylene glycol, polypropylene glycol, thiol, or the like);

Q₇ is a residue selected from the group consisting of residues which are hydrophilic at physiologic pH, and are at least partially positively charged at about neutral pH (e.g., amino, alkylamino, ammonium, alkylammonium, guanidine, imidazolyl, pyridyl, or the like); at least partially negatively charged at about neutral pH but undergo protonation at lower pH (e.g., carboxyl, sulfonamide, boronate, phosphonate, phosphate, or the like); at least partially zwitterionic at physiologic pH (e.g., a monomeric residue comprising a phosphate group and an ammonium group at physiologic pH), or hydrogen;

Q₈ is selected from a group consisting of a conjugatable or functionizable residue (e.g. residues that comprise a reactive group, e.g., azide, alkyne, succinimide ester, tetrafluorophenyl ester, pentafluorophenyl ester, p-nitrophenyl ester, pyridyl disulfide, aldehyde, ketone, amine, or the like) or a targeting agent (e.g., sugar residue, folate, peptide, or the like);

g is 0 to 1;

h is 0 to less than 1;

k is 0 to 1;

wherein g+h+k=1; and

y is about 1 to about 100 kDa, or about 1 to about 30 kDa.

In some embodiments, the hydrophilic block has a structure corresponding to Formula Ia-2:

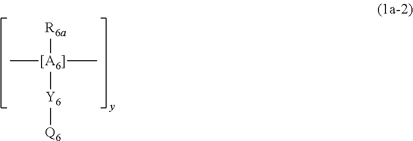

wherein:

A₆ is selected from the group consisting of —C—C—, —C—, —C(O)(C)ₐC(O)O—, —O(C)ₐC(O)— and —O(C)_{b}O—;

a is 1-4;

b is 2-4;

R⁴ is selected from the group consisting of hydrogen, and optionally substituted C₁-C₆ alkyl,

Y₆ is selected from the group consisting of a covalent bond, (1C-10C)alkyl-, (3C-6C)cycloalkyl, O-(1C-10C)alkyl, —C(O)O(2C-10C) alkyl-, —OC(O)(1C-10C) alkyl-, —O(2C-10C)alkyl-, —S(2C-10C)alkyl-, —C(O)NR₆(2C-10C) alkyl-, and aryl, any of which is optionally substituted;

tetravalent carbon atoms of A₆ that are not fully substituted with R₆ₐ and Y₆ is completed with an appropriate number of hydrogen atoms;

each R₆ₐ is independently selected from the group consisting of hydrogen, —CN, alkyl, alkynyl, heteroalkyl, cycloalkyl, heterocycloalkyl, aryl and heteroaryl, any of which may be optionally substituted with one or more fluorine atoms;

Q₆ is a residue selected from the group consisting of residues which are hydrophilic at physiologic pH and are substantially non-charged at physiologic pH (e.g., hydroxy, polyoxylated alkyl, polyethylene glycol, polypropylene glycol, thiol, or the like); and

y is about 1 to about 30 kDa.

In some embodiments, the hydrophilic block is a copolymer comprising monomeric residues corresponding to Formula Ia, and other monomeric residues. In preferred embodiments, the other monomeric residues are non-charged. In some embodiments, the other monomeric residues are non-hydrophobic. In certain embodiments, the other monomeric residues do not significantly affect the overall hydrophilicity of the hydrophilic block.

In one preferred embodiment, the hydrophilic block comprises repeat units corresponding to Formula 1

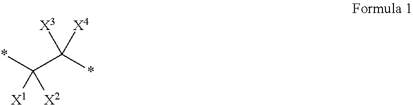

wherein * designates the point of attachment of the repeat unit of Formula 1 to other repeat units; each X¹ and X² is independently selected from the group consisting of hydrogen, hydrocarbyl, substituted hydrocarbyl, heterocyclo, and substituted carbonyl, provided, however, X¹ and X² are not, in the same repeat unit, selected from the group consisting of aryl, heteroaryl, heterosubstituted carbonyl, and combinations thereof; each X³ is independently hydrogen, alkyl or substituted alkyl, and each X⁴ is independently heterosubstituted carbonyl, aryl, or heteroaryl. For example, in one such embodiment, the hydrophilic block comprises repeat units corresponding to Formula 1 and X¹ and X² are each hydrogen. In another such example, the hydrophilic block comprises repeat units corresponding to Formula 1, X¹ and X² are each hydrogen and X³ is hydrogen or alkyl. In a further example, the hydrophilic block comprises repeat units corresponding to Formula 1, X¹ and X² are each hydrogen, X³ is hydrogen or alkyl, and each X⁴ is independently heterosubstituted carbonyl. In a further example, the hydrophilic block comprises repeat units corresponding to Formula 1, X⁴ is —C(O)OX⁴⁰, —C(O)SX⁴⁰, or —C(O)NX⁴⁰X⁴¹, and X⁴⁰ and X⁴¹ are independently hydrogen, hydrocarbyl, substituted hydrocarbyl, heterohydrocarbyl, substituted heterohydrocarbyl, or heterocyclo. In a further example, the hydrophilic block comprises repeat units corresponding to Formula 1, X⁴ is —C(O)OX⁴⁰ or —C(O)NX⁴⁰X⁴¹, and X⁴⁰ and X⁴¹ are independently hydrogen, hydrocarbyl, substituted hydrocarbyl, heterohydrocarbyl, substituted heterohydrocarbyl, or heterocyclo. In a further example, the hydrophilic block comprises repeat units corresponding to Formula 1, X¹ and X² are each hydrogen, X³ is hydrogen or alkyl, X⁴ is —C(O)OX⁴⁰, and X⁴⁰ is hydrogen, hydrocarbyl, substituted hydrocarbyl, heterohydrocarbyl, substituted heterohydrocarbyl, or heterocyclo. In a further example, the hydrophilic block comprises repeat units corresponding to Formula 1, X¹ and X² are each hydrogen, X³ is hydrogen or alkyl, X⁴ is —C(O)OX⁴⁰, X⁴⁰ is —(CH₂CH₂O)ₜX⁴⁰⁰, t is a positive integer, and X⁴⁰⁰ is alkyl, substituted alkyl, or heterocyclo. In a further example, the hydrophilic block comprises repeat units corresponding to Formula 1, X¹ and X² are each hydrogen, X³ is hydrogen or alkyl, X⁴ is —C(O)OX⁴⁰, X⁴⁰ is —(CH₂CH₂O)ₜX⁴⁰⁰, t is a positive integer, and X⁴⁰⁰ comprises a targeting moiety. In a further example, the hydrophilic block comprises repeat units corresponding to Formula 1 and the repeat units corresponding to Formula 1 constitute a majority of the total number of repeat units in the hydrophilic block. In a further example, the hydrophilic block comprises repeat units corresponding to Formula 1 and the repeat units corresponding to Formula 1 constitute at least 75% of the total number of repeat units in the hydrophilic block. In a further example, the hydrophilic block comprises repeat units corresponding to Formula 1 and the repeat units corresponding to Formula 1 constitute at least 90% of the total number of repeat units in the hydrophilic block.

In one alternative embodiment, the hydrophilic block is a random copolymer comprising at least two compositionally distinct repeat units, at least one of which corresponds to Formula 1. In another alternative embodiment, the hydrophilic block is a random copolymer comprising at least two compositionally distinct repeat units, each of which corresponds to Formula 1. For example, the hydrophilic block may be a random copolymer comprising (i) a first repeat unit corresponding to Formula 1 in which X⁴ is —C(O)OH and (ii) a compositionally distinct second repeat unit corresponding to Formula 1. In a further example, the hydrophilic block is a random copolymer comprising at least two compositionally distinct repeat units, each of which corresponds to Formula 1, and the repeat units corresponding to Formula 1 constitute a majority of the total number of repeat units in the hydrophilic block. In a further example, the hydrophilic block is a random copolymer comprising at least two compositionally distinct repeat units, each of which corresponds to Formula 1, and the repeat units corresponding to Formula 1 constitute at least 90% of the total number of repeat units in the hydrophilic block.

In another preferred embodiment, the hydrophilic block comprises repeat units corresponding to Formula 1ETS

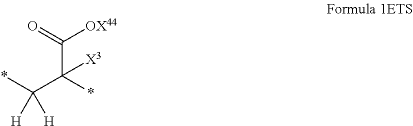

wherein * designates the point of attachment of the repeat unit of Formula 1ETS to other repeat units, X³ is alkyl, and X⁴⁴ is a targeting or shielding moiety. For example, X⁴⁴ may be a polyol, a vitamin, a peptide, or other moiety that has a binding affinity for a cellular or biological target. For example, in one such embodiment the hydrophilic block comprises repeat units corresponding to Formula 1ETS and X⁴⁴ is a polyol. In a further example, the hydrophilic block comprises repeat units corresponding to Formula 1ETS and the repeat units corresponding to Formula 1ETS may constitute at least 2% of the total number of repeat units in the hydrophilic block. In a further example, the hydrophilic block comprises repeat units corresponding to Formula 1ETS and the repeat units corresponding to Formula 1ETS may constitute at least 5% of the total number of repeat units in the hydrophilic block. In a further example, the hydrophilic block is a random copolymer comprising at least two repeat units, one corresponding to Formula 1ETS and the other being a compositionally distinct repeat unit corresponding to Formula 1. In a further example, the hydrophilic block is a random copolymer comprising at least two repeat units, one corresponding to Formula 1ETS and another corresponding to Formula 1 wherein X⁴ is —COOH.

In certain embodiments, the hydrophilic block comprises a plurality of monomeric residues obtained by the polymerization or copolymerization of a monomer of Formula IIIb:

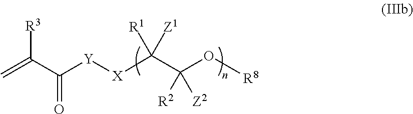

wherein:

n is an integer ranging from 2 to 20;

X is —(CR¹R²)ₘ— wherein m is 0-10, and wherein one or more (CR¹R²) unit is optionally substituted with —NR¹R², —OR¹ or —SR¹,

Y is —O—, —NR⁴— or —(CR¹R²)—,

each R¹, R², R³, Z¹ and Z² are independently selected from the group consisting of hydrogen, halogen, and optionally substituted C₁-C₃ alkyl,

R⁴ is selected from the group consisting of hydrogen, and optionally substituted C₁-C₆ alkyl,

R⁸ is hydrogen or (CR¹R²)ₘR⁹, wherein m is 0-10, and wherein one or more (CR¹R²) unit is optionally substituted with —NR¹R², —OR¹ or —SR¹, and

R⁹ is hydrogen, halogen, optionally substituted C₁-C₃ alkyl, polyol, vitamin, peptide, small molecule having a molecular weight of 200-1200 Daltons, or a conjugatable group.

In certain embodiments, the hydrophilic block comprises a plurality of monomeric residues obtained by the polymerization or copolymerization of a monomer of Formula IIIc:

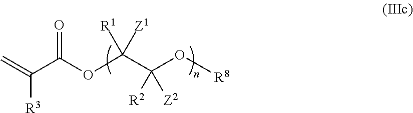

wherein:

n is an integer ranging from 2 to 20;

X is —(CR¹R²)ₘ— wherein m is 0-10, and wherein one or more (CR¹R²) unit is optionally substituted with —NR¹R², —OR¹ or —SR¹,

Y is —O—, —NR⁴— or —(CR¹R²)—,

each R¹, R², R³, Z¹ and Z² are independently selected from the group consisting of hydrogen, halogen, and optionally substituted C₁-C₃ alkyl,

R⁴ is selected from the group consisting of hydrogen, and optionally substituted C₁-C₆ alkyl,

R⁸ is hydrogen or (CR¹R²)ₘR⁹, wherein m is 0-10, and wherein one or more (CR¹R²) unit is optionally substituted with —NR¹R², —OR¹ or —SR¹, and

R⁹ is hydrogen, halogen, optionally substituted C₁-C₃ alkyl, polyol, vitamin, peptide, small molecule having a molecular weight of 200-1200 Daltons, or a conjugatable group.

For example, in one embodiment, the hydrophilic block comprises a plurality of monomeric residues corresponding to Formula IIIc, R¹, R², Z¹ and Z² are hydrogen, R³ is hydrogen or C₁-C₃ alkyl, n is 2-20 and R⁹ is a polyol, vitamin, peptide, or small molecule having a molecular weight of 200-1200 Daltons.

In certain embodiments, the hydrophilic block comprises a plurality of monomeric residues obtained by the polymerization or copolymerization of a monomer of Formula III:

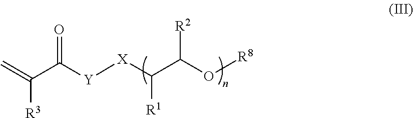

wherein:

X is (CR¹R²)ₘ, wherein m is 0-10, and wherein one or more (CR¹R²) unit is optionally substituted with NR¹R², OR¹ or SR¹,

Y is O, NR⁴, or CR¹R²,

each R¹, R², R³ and R⁹ are independently selected from the group consisting of hydrogen, halogen, optionally substituted C₁-C₃ alkyl, or a targeting group, such as but not limited to galactose, N-acetyl galactosamine, folate, RGD peptide,

R⁴ is selected from the group consisting of hydrogen, and optionally substituted C₁-C₆ alkyl,

n is an integer ranging from 2 to 20,

R⁸ is (CR¹R²)ₘR⁹, wherein m is 0-10, and wherein one or more (CR¹R²) unit is optionally substituted with NR¹R², OR¹ or SR¹.

In specific embodiments, the hydrophilic block comprises a plurality of monomeric residues obtained by the polymerization or copolymerization of a monomer of Formula IIIa:

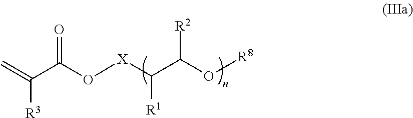

wherein:

X is (CR¹R²)ₘ, wherein m is 0-10, and wherein one or more (CR¹R²) unit is optionally substituted with NR¹R², OR¹ or SR¹,

each R¹, R², R³ and R⁹ are independently selected from the group consisting of hydrogen, halogen, and optionally substituted C₁-C₃ alkyl,

n is an integer ranging from 2 to 20,

R⁸ is (CR¹R²)ₘR⁹, wherein m is 0-10, and wherein one or more (CR¹R²) unit is optionally substituted with NR¹R², OR¹ or SR¹.

In one embodiment, the hydrophilic block comprises a plurality of monomeric residues corresponding to Formula IIIa and R³ is methyl (i.e., the monomer of Formula III is a PEGMA).

In certain embodiments, the hydrophilic block comprises a plurality of monomeric residues obtained by the polymerization or copolymerization of a monomer of Formula IV:

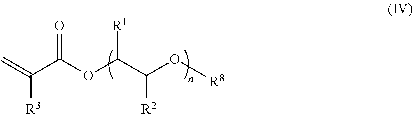

wherein:

each R¹, R² and R³ are independently selected from the group consisting of hydrogen, halogen, and optionally substituted C₁-C₃ alkyl,

n is an integer ranging from 2 to 20,

R⁸ is (CR¹R²)ₘR⁹, wherein m is 0-10, and wherein one or more (CR¹R²) unit is optionally substituted with NR¹R², OR¹ or SR¹.

In one embodiment, the hydrophilic block comprises a plurality of monomeric residues corresponding to Formula IV, R³ is methyl and n is 2-20.

In general, the hydrophilic block comprises a plurality of repeat units, i.e., at least two. In some embodiments, a hydrophilic block of a polymer described herein has a number average molecular weight of about 1,000 Dalton to about 50,000 Dalton, about 2,000 Dalton to about 30,000 Dalton, about 5,000 Dalton to about 20,000 Dalton, or about 7,000 Dalton to about 15,000 Dalton. In specific embodiments, the hydrophilic block is of about 7,000 Dalton, 8,000 Dalton, 9,000 Dalton, 10,000 Dalton, 11,000 Dalton, 12,000 Dalton, 13,000 Dalton, 14,000 Dalton, or 15,000 Dalton.

Block Ratios

In certain embodiments, the polymer of the present invention is a block copolymer comprising a hydrophilic and a hydrophobic block. In this embodiment, the block copolymer has a ratio of a number-average molecular weight (with the number average molecular weight of the first block, i.e., hydrophilic block, represented by Mₙ^{1st}, the number average molecular weight of the hydrophobic block represented by Mₙ^{2nd}, of Mₙ^{1st}):(Mₙ^{2nd}) of about 2:1 to about 1:9. In some embodiments, (Mₙ^{1st}):(Mₙ^{2nd}) is about 1:1 to about 1:3.

Polymerization

In certain embodiments, the hydrophobic and hydrophilic blocks of the block copolymer comprise monomeric residues derived from a polymerizable monomer. As noted previously, the block copolymer may also comprise one or more other blocks in addition to the hydrophobic block and, in those instances, at least one of the additional blocks may also comprise monomeric residues derived from a polymerizable monomer. In specific embodiments, any of the monomers polymerized or copolymerized to provide the hydrophilic or hydrophobic blocks include an ethylenically unsaturated monomer. In more specific embodiments, ethylenically unsaturated monomers include, by way of non-limiting example, acrylic monomers, a vinylic monomer, and the like having at least one carbon double or triple bond. Non-limiting examples of ethylenically unsaturated monomers include an alkyl (alkyl)acrylate, a methacrylate, an acrylate, an alkylacrylamide, a methacrylamide, an acrylamide, a styrene, an allylamine, an allylammonium, a diallylamine, a diallylammonium, an N-vinyl formamide, a vinyl ether, a vinyl sulfonate, an acrylic acid, a sulfobetaine, a carboxybetaine, a phosphobetaine, or maleic anhydride In some embodiments, the ethylenically unsaturated monomer is an acrylic monomer selected from an optionally substituted acrylic acid, an optionally substituted acrylamide, and an optionally substituted acrylate. In certain embodiments, the ethylenically unsaturated monomer is selected from optionally C₁-C₈ alkyl-substituted acrylic acid, an optionally C₁-C₈ alkyl-substituted acrylamide, and an optionally C₁-C₈ alkyl-substituted acrylate.

In various embodiments, any monomer suitable for providing membrane destabilizing block copolymers may be used. In some embodiments, monomers suitable for use in the preparation of the polymers (including, e.g., the membrane destabilizing block copolymers) include, by way of non-limiting example, one or more of the following monomers: methyl methacrylate, ethyl acrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, alpha-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, acrylates and styrenes selected from glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N-n-butylmethacrylamide, N-methylolacrylamide, N-ethylolacrylamide, vinyl benzoic acid (all isomers), diethylaminostyrene (all isomers), alpha-methylvinyl benzoic acid (all isomers), diethylamino alpha-methylstyrene (all isomers), p-vinylbenzenesulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropylmethacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, triethoxysilylpropyl acrylate, tributoxysilylpropyl acrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, vinyl acetate, vinyl butyrate, vinyl benzoate, vinyl chloride, vinyl fluoride, vinyl bromide, maleic anhydride, N-arylmaleimide, N-phenylmaleimide, N-alkylmaleimide, N-butylimaleimide, N-vinylpyrrolidone, N-vinylcarbazole, butadiene, isoprene, chloroprene, ethylene, propylene, 1,5-hexadienes, 1,4-hexadienes, 1,3-butadienes, 1,4-pentadienes, vinylalcohol, vinylamine, N-alkylvinylamine, allylamine, N-alkylallylamine, diallylamine, N-alkyldiallylamine, alkylenimine, acrylic acids, alkylacrylates, acrylamides, methacrylic acids, alkylmethacrylates, methacrylamides, N-alkylacrylamides, N-alkylmethacrylamides, styrene, vinylnaphthalene, vinyl pyridine, ethylvinylbenzene, aminostyrene, vinylpyridine, vinylimidazole, vinylbiphenyl, vinylanisole, vinylimidazolyl, vinylpyridinyl, vinylpolyethyleneglycol, dimethylaminomethylstyrene, trimethylammonium ethyl methacrylate, trimethylammonium ethyl acrylate, dimethylamino propylacrylamide, trimethylammonium ethylacrylate, trimethylanunonium ethyl methacrylate, trimethylammonium propyl acrylamide, dodecyl acrylate, octadecyl acrylate, or octadecyl methacrylate monomers, or combinations thereof.

In some embodiments, functionalized versions of these monomers are optionally used. A functionalized monomer, as used herein, is a monomer comprising a masked or non-masked functional group, e.g. a group to which other moieties can be attached following the polymerization. The non-limiting examples of such groups are primary amino groups, carboxyls, thiols, hydroxyls, azides, and cyano groups. Several suitable masking groups are available (see, e.g., T. W. Greene & P. G. M. Wuts, Protective Groups in Organic Synthesis (2nd edition) J. Wiley & Sons, 1991. P. J. Kocienski, Protecting Groups, Georg Thieme Verlag, 1994).

Polymers described here are prepared in any suitable manner. Suitable synthetic methods used to produce the polymers provided herein include, by way of non-limiting example, cationic, anionic and free radical polymerization. In some instances, when a cationic process is used, the monomer is treated with a catalyst to initiate the polymerization. Optionally, one or more monomers are used to form a copolymer. In some embodiments, such a catalyst is an initiator, including, e.g., protonic acids (Bronsted acid) or Lewis acids, in the case of using Lewis acid some promoter such as water or alcohols are also optionally used. In some embodiments, the catalyst is, by way of non-limiting example, hydrogen iodide, perchloric acid, sulfuric acid, phosphoric acid, hydrogen fluoride, chlorosulfonic acid, methansulfonic acid, trifluoromehtanesulfonic acid, aluminum trichloride, alkyl aluminum chlorides, boron trifluoride complexes, tin tetrachloride, antimony pentachloride, zinc chloride, titanium tetrachloride, phosphorous pentachloride, phosphorus oxychloride, or chromium oxychloride. In certain embodiments, polymer synthesis is performed neat or in any suitable solvent. Suitable solvents include, but are not limited to, pentane, hexane, dichloromethane, chloroform, or dimethyl formamide (DMF). In certain embodiments, the polymer synthesis is performed at any suitable reaction temperature, including, e.g., from about −50° C. to about 100° C., or from about 0° C. to about 70° C.

In certain embodiments, the polymers are prepared by the means of a free radical polymerization. When a free radical polymerization process is used, (i) the monomer, (ii) optionally, the co-monomer, and (iii) an optional source of free radicals are provided to trigger a free radical polymerization process. In some embodiments, the source of free radicals is optional because some monomers may self-initiate upon heating at high temperature. In certain instances, after forming the polymerization mixture, the mixture is subjected to polymerization conditions. Polymerization conditions are those conditions that cause at least one monomer to form at least one polymer, as discussed herein. Such conditions are optionally varied to any suitable level and include, by way of non-limiting example, temperature, pressure, atmosphere, ratios of starting components used in the polymerization mixture and reaction time. The polymerization is carried out in any suitable manner, including, e.g., in solution, dispersion, suspension, emulsion or bulk.

In some embodiments, initiators are present in the reaction mixture. Any suitable initiator is optionally utilized if useful in the polymerization processes described herein. Such initiators include, by way of non-limiting example, one or more of alkyl peroxides, substituted alkyl peroxides, aryl peroxides, substituted aryl peroxides, acyl peroxides, alkyl hydroperoxides, substituted alkyl hydroperoxides, aryl hydroperoxides, substituted aryl hydroperoxides, heteroalkyl peroxides, substituted heteroalkyl peroxides, heteroalkyl hydroperoxides, substituted heteroalkyl hydroperoxides, heteroaryl peroxides, substituted heteroaryl peroxides, heteroaryl hydroperoxides, substituted heteroaryl hydroperoxides, alkyl peresters, substituted alkyl peresters, aryl peresters, substituted aryl peresters, or azo compounds. In specific embodiments, benzoylperoxide (BPO) and/or AIBN are used as initiators.

In some embodiments, polymerization processes are carried out in a living mode, in any suitable manner, such as but not limited to Atom Transfer Radical Polymerization (ATRP), nitroxide-mediated living free radical polymerization (NMP), ring-opening polymerization (ROP), degenerative transfer (DT), or Reversible Addition Fragmentation Transfer (RAFT). Using conventional and/or living/controlled polymerizations methods, various polymer architectures can be produced, such as but not limited to block, graft, star and gradient copolymers, whereby the monomer units are either distributed statistically or in a gradient fashion across the chain or homopolymerized in block sequence or pendant grafts. In other embodiments, polymers are synthesized by Macromolecular design via reversible addition-fragmentation chain transfer of Xanthates (MADIX) (“Direct Synthesis of Double Hydrophilic Statistical Di- and Triblock Copolymers Comprised of Acrylamide and Acrylic Acid Units via the MADIX Process”, Daniel Taton, et al., Macromolecular Rapid Communications, 22, No. 18, 1497-1503 (2001)).

In certain embodiments, Reversible Addition-Fragmentation chain Transfer or RAFT is used in synthesizing ethylenic backbone polymers of this invention. RAFT is a living polymerization process. RAFT comprises a free radical degenerative chain transfer process. In some embodiments, RAFT procedures for preparing a polymer described herein employs thiocarbonylthio compounds such as, without limitation, dithioesters, dithiocarbamates, trithiocarbonates and xanthates to mediate polymerization by a reversible chain transfer mechanism. In certain instances, reaction of a polymeric radical with the C═S group of any of the preceding compounds leads to the formation of stabilized radical intermediates. Typically, these stabilized radical intermediates do not undergo the termination reactions typical of standard radical polymerization but, rather, reintroduce a radical capable of re-initiation or propagation with monomer, reforming the C═S bond in the process. In most instances, this cycle of addition to the C═S bond followed by fragmentation of the ensuing radical continues until all monomer has been consumed or the reaction is quenched. Generally, the low concentration of active radicals at any particular time limits normal termination reactions.

In some embodiments, polymers of the present invention have a low polydispersity index (PDI) or differences in chain length. Polydispersity index (PDI) can be determined in any suitable manner, e.g., by dividing the weight average molecular weight of the polymer chains by their number average molecular weight. The number average molecule weight is sum of individual chain molecular weights divided by the number of chains. The weight average molecular weight is proportional to the square of the molecular weight divided by the number of molecules of that molecular weight. Since the weight average molecular weight is always greater than the number average molecular weight, polydispersity is always greater than or equal to one. As the numbers come closer and closer to being the same, i.e., as the polydispersity approaches a value of one, the polymer becomes closer to being monodisperse in which every chain has exactly the same number of constitutional units. Polydispersity values approaching one are achievable using radical living polymerization. Methods of determining polydispersity, such as, but not limited to, size exclusion chromatography, dynamic light scattering, matrix-assisted laser desorption/ionization chromatography and electrospray mass chromatography are well known in the art. In some embodiments, the polymers (e.g., membrane destabilizing polymers) provided herein have a polydispersity index (PDI) of less than 2.0, or less than 1.8, or less than 1.6, or less than 1.5, or less than 1.4, or less than 1.3, or less than 1.2. In some embodiments, the polymer is a block copolymer (e.g., membrane destabilizing block copolymers) comprising a hydrophilic block and a hydrophobic block and having a polydispersity index (PDI) of less than 2.0, or less than 1.8, or less than 1.6, or less than 1.5, or less than 1.4, or less than 1.3, or less than 1.2.

Polymerization processes described herein optionally occur in any suitable solvent or mixture thereof. Suitable solvents include water, alcohol (e.g., methanol, ethanol, n-propanol, isopropanol, butanol), tetrahydrofuran (THF) dimethyl sulfoxide (DMSO), dimethylformamide (DMF), acetone, acetonitrile, hexamethylphosphoramide, acetic acid, formic acid, hexane, cyclohexane, benzene, toluene, dioxane, methylene chloride, ether (e.g., diethyl ether), chloroform, and ethyl acetate. In one aspect, the solvent includes water, and mixtures of water and water-miscible organic solvents such as DMF.

In certain embodiments, poly(DMAEMA) and other polymeric entities used herein (e.g., copolymers or copolymer blocks of BMA, DMAEMA and PAA) are prepared in any suitable manner. In one embodiment, poly(DMAEMA/PEGMA) is prepared by co-polymerizing DMAEMA and PEGMA in the presence of the RAFT CTA, ECT, and a radical initiator. In some embodiments, a block, poly(DMAEMA/PEGMA) macroCTA is used to prepare a series of diblock, triblock or other multiblock copolymers where the hydrophobic block contains BMA, DMAEMA and PAA. In other specific embodiments, the orientation of the blocks on the diblock, triblock or other multiblock polymer is reversed, such that upon self-assembly, the w end of the polymer is exposed on the hydrophilic segment of the micelle. In various embodiments, this is achieved in any suitable manner, including a number of ways synthetically. For example, in some embodiments, the synthesis of the block copolymers described herein begins with the preparation of the PAA/BMA/DMAEMA core-forming hydrophobic block, and the shell-forming hydrophilic, charged block is added in the second synthetic step by subjecting the resulting PAA/BMA/DMAEMA macroCTA to a second RAFT polymerization step. Alternate approaches include reducing the PAA/BMA/DMAEMA macroCTA to form a thiol end and then covalently attaching a pre-formed hydrophilic, charged polymer to the formed thiol. This synthetic approach provides a method for introduction of a reactive group on the omega-end of the polymeric chain exposed to the surface of micelle thus providing alternate approaches to chemical conjugation to the micelle.

In some embodiments, block copolymers are synthesized by chemical conjugation of several polymer blocks that are prepared by separate polymerization processes.

In certain embodiments, the ethylenically unsaturated monomer is selected from, by way of non-limiting example:

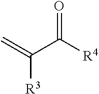

wherein

R³ is hydrogen, halogen, hydroxyl, or optionally substituted C₁-C₃ alkyl;

R⁴ is —SR⁵, —OR⁵, —NR⁶R⁷, or

R⁴ is C₁-C₄₀ alkyl or C₁-C₄₀ polyoxylatedalkyl, optionally substituted by halogen, cyano, hydroxyl, alkoxy, thiol, alkylthio, silylalkyl, silylaryl, —NR⁹R¹⁰, cycloalkyl, heterocycloalkyl, —C(═O)OR⁹, —S(═O)OR⁹, —S(═O)₂OR⁹, —C(═O)NR⁹R¹⁰, —S(═O)NR⁹R¹⁰, —S(═O)₂NR⁹R¹⁰, a cleavable group or a functionizable group;

R⁵ is C₁-C₄₀ alkyl, C₁-C₄₀ polyoxylatedalkyl, C₁-C₄₀ alkenyl, C₁-C₄₀ alkynyl, cycloalkyl, heterocycloalkyl, aryl, or heteroaryl, optionally substituted by halogen, cyano, hydroxyl, alkoxy, thiol, alkylthio, silylalkyl, silylaryl, —NR⁹R¹⁰, cycloalkyl, heterocycloalkyl, —C(═O)OR⁹, —S(═O)OR⁹, —S(═O)₂OR⁹, —C(═O)NR⁹R¹⁰, —S(═O)NR⁹R¹⁰, —S(═O)₂NR⁹R¹⁰, a cleavable group or a functionizable group;

R⁶ and R⁷ are each independently hydrogen, C₁-C₄₀ alkyl, C₁-C₄₀ polyoxylatedalkyl, C₁-C₄₀ alkenyl, C₁-C₄₀ alkynyl, cycloalkyl, heterocycloalkyl, aryl, or heteroaryl, optionally substituted by halogen, cyano, hydroxyl, alkoxy, thiol, alkylthio, silylalkyl, silylaryl, —NR⁹R¹⁰, cycloalkyl, heterocycloalkyl, —C(═O)OR⁹, —S(═O)OR⁹, —S(═O)₂OR⁹, —C(═O)NR⁹R¹⁰, —S(═O)NR⁹R¹⁰, —S(═O)₂NR⁹R¹⁰, a cleavable group or a functionizable group; or

R⁶ and R⁷ together with the nitrogen to which they are attached form an optionally substituted heterocycle;

R⁹ and R¹⁰ are each independently hydrogen, optionally substituted C₁-C₆ alkyl, optionally substituted cycloalkyl, optionally substituted aryl, optionally substituted heteroaryl, or

R⁹ and R¹⁰ together with the nitrogen to which they are attached form a heterocycle.

Micelle

In certain embodiments, micelles are formed from a plurality of polymers comprising a hydrophilic block and a hydrophobic block as described elsewhere herein. In certain embodiments, a micelle described herein comprises a plurality of block copolymers, the micelle comprising a core and a shell. In specific embodiments, the core of the micelle comprises the hydrophobic blocks of the plurality of block copolymers and the shell comprises the hydrophilic blocks of the block copolymers. Moreover, in more specific embodiments, the shell comprises an inner layer and an outer layer (relative to one another; further layers are also possible), the hydrophilic block forming the outer layer and the second hydrophilic block forming the inner layer of the micelle shell.

In specific embodiments, a plurality of any one or more polymer described herein is assembled into a micelle. In specific embodiments, such a micelle is stable in at about neutral pH (e.g., is stable in an aqueous medium at about pH 7.4).

In some embodiments, a micelle described herein comprises any number of polymers described herein, e.g., about 10 to about 100 of the copolymers described herein per micelle, or about 20 to about 60 of the copolymers described herein per micelle.

In one embodiment, a micelle described herein (without an associated therapeutic agent such as a polynucleotide) has a Zeta potential that is between ±6 mV (millivolt). In one preferred embodiment, a micelle described herein (without an associated therapeutic agent such as a polynucleotide) has a Zeta potential that is between ±5 mV. In one preferred embodiment, a micelle described herein (without an associated therapeutic agent such as a polynucleotide) has a Zeta potential that is between ±2 mV.

In general, the micelles described herein may have a critical micelle concentration, CMC, ranging from about 0.2 μg/ml to about 100 μg/ml. For example, in one preferred embodiment, the micelles have a CMC of about 0.2 μg/ml to about 20 μg/ml. In specific embodiments, the micelle has a critical micelle concentration, CMC, ranging from about 0.5 μg/ml to about 10 μg/ml. In certain embodiments, a micelle described herein comprises a plurality of block copolymers described herein, the plurality of block copolymers described herein having a polydispersity index (PDI) of about 1.1 to about 1.7, or about 1.1 to about 1.4. In some embodiments, a micelle comprises a plurality of copolymers described herein wherein the hydrophobic block(s) and any hydrophilic block(s), when present, of the block copolymer have a polydispersity index of not more than 1.5.

In certain embodiments, a micelle described herein comprise a plurality of copolymers, as described herein, wherein at least one or more of the plurality of copolymers is covalently crosslinked to the second polymer, whereby the polymeric micelle is a crosslinked polymeric micelle. In specific embodiments, the second polymer is also a copolymer as described herein. In some embodiments, the block copolymer is covalently crosslinked to the hydrophobic block of the second polymer. In specific embodiments, the hydrophobic blocks of two copolymers described herein are cross-linked to each other. In certain embodiments, the block copolymer comprises a plurality of monomeric residues derived from controlled radical polymerization of an ethylenic monomer, at least one such monomer being a bis-functional crosslinking monomer.

In some embodiments, a micelle provided herein is characterized by one or more of the following: (1) the micelle is formed by spontaneous self association of block copolymers to form organized assemblies (e.g., micelles) upon dilution from a water-miscible solvent (such as but not limited to ethanol) to aqueous solvents (for example phosphate-buffered saline, pH 7.4); (2) the micelle is stable to dilution (e.g., down to a polymer concentration of 100 ug/ml, 50 ug/ml, 10 ug/ml, 5 ug/ml or 1 ug/ml, which constitutes the critical stability concentration or the critical micelle concentration (CMC)); (3) the micelle is stable to high ionic strength of the surrounding media (e.g. 0.5M NaCl); and/or (4) the micelle has an increasing instability as the concentration of organic solvent increases, such organic solvents including, but not limited to dimethylformamide (DMF), dimethylsulfoxide (DMSO), and dioxane. In some embodiments, a micelle provided herein is characterized by having at least two of the aforementioned properties. In some embodiments, a micelle provided herein is characterized by having at least three of the aforementioned properties. In some embodiments, a micelle provided herein is characterized by having all of the aforementioned properties.

In some embodiments, a micelle provided herein self-assembles at any suitable concentration. In certain embodiments, a micelle provided herein self-assembles (e.g., has a critical micelle concentration (CMC), or the minimum concentration at which a micelle forms) of about 2 μg/mL, about 5 μg/mL, about 8 μg/mL, about 10 μg/mL, about 20 μg/mL, about 25 μg/mL, about 30 μg/mL, about 40 μg/mL, about 50 μg/mL, about 60 μg/mL, about 70 μg/mL, about 80 μg/mL, about 90 μg/mL, about 100 μg/mL, or greater. In certain embodiments, a micelle provided herein self assembles at least one concentration between about 1 μg/mL and about 100 μg/mL.

In some embodiments, the micelles provided herein are prepared by spontaneous self-assembly of the polymers described herein. In certain embodiments, the polymers described herein assemble into the micelles provided herein upon dilution of a solution of the polymer in water-miscible organic solvent into aqueous media. In some embodiments, the micelles provided herein are formed spontaneously upon dissolution of the polymer directly in aqueous media. In some embodiments, the micelles do not require the presence of a polynucleotide for micelle formation.

In some embodiments, the micelles are stable to dilution in an aqueous solution. In specific embodiments, the micelles are stable to dilution at physiologic pH (including the pH of circulating blood in a human) with a critical stability concentration (e.g., a critical micelle concentration (CMC)) of approximately 50 to approximately 100 μg/mL, or approximately 10 to approximately 50 μg/mL, or less than 10 μg/mL. For example, in a preferred embodiment, the average hydrodynamic particle size for the micelle, as determined by dynamic light scattering techniques, does not change more than approximately 30% within the 5 minute period following dilution from a greater to a lesser concentration in an aqueous solution at a pH of 7.4, preferably at 37° C., the lesser concentration being greater than the midpoint polymer concentration of the transitional change following the concentration at which the micelle forms, as determined by uptake of a hydrophobic probe molecule (for example, the pyrene fluorescence assay).

In some embodiments, a micelle provided herein is stable in an aqueous medium. In certain embodiments, a micelle provided herein is stable in an aqueous medium at a selected pH, e.g., about physiological pH (e.g., the pH of circulating human plasma). In specific embodiments, a micelle provided herein is stable at about a neutral pH (e.g., at a pH of about 7.4) in an aqueous medium. In specific embodiments, the aqueous medium is animal (e.g., human) serum or animal (e.g., human) plasma. In certain embodiments, a micelle provided herein is stable in human serum and/or human plasma. In specific embodiments, the micelle is stable in circulating human plasma. It is to be understood that stability of the micelle is not limited to designated pH, but that it is stable at pH values that include, at a minimum, the designated pH. In specific embodiments, a micelle described herein is substantially less stable at an acidic pH than at a pH that is about neutral. In more specific embodiments, a micelle described herein is substantially less stable at a pH of about 5.8 than at a pH of about 7.4.

In specific embodiments, the micelle is stable at a concentration of about 10 μg/mL, or greater (e.g., at about a neutral pH). In some embodiments, the micelle is stable at a concentration of about 100 μg/mL, or greater (e.g., at about a neutral pH).

In one embodiment, the micelle is a heterogeneous polymeric micelle comprising a polymer of the present invention and at least one additional compositionally distinct polymer. Other compositionally distinct polymers include, for example, other block copolymers comprising a hydrophilic block and a hydrophobic block. Advantageously, the hydrophobic block of the other block copolymer(s) can associate with the hydrophobic block of the polymer of the present invention through hydrophobic interactions to form a hydrophobic core. By way of example, the heterogeneous micelle comprises (i) polymers having a therapeutic agent covalently coupled to a hydrophobic block thereof (the polymer optionally additionally comprising a hydrophilic block) and (ii) a multiblock copolymer comprising hydrophilic block(s) and hydrophobic block(s) but having no therapeutic agent covalently coupled thereto. Preferably, the heterogeneous micelle is stable in aqueous medium at a physiologically relevant pH (e.g., pH 7.4). In some embodiments, the heterogeneous polymeric micelle comprises one or more additional compositionally distinct polymers, such as a third polymer that is compositionally distinct from each of the first polymer and the second polymer. Generally, each block of a block copolymer (e.g., of the first polymer and/or the second polymer) can be a homopolymer or a random copolymer, in each case linear or non linear (e.g., branched), and in each case crosslinked or uncrosslinked, and can generally comprise one or more monomeric residues derived from polymerization of a polymerizable monomer (e.g., using controlled living radical polymerization approaches).

Targeting

In certain embodiments, polymers or micelles described herein comprise at least one targeting moiety (e.g., a moiety that targets a specific cell or type of cell). In specific instances, the polymers and micelles provided herein are useful for delivery of therapeutic agents to specifically targeted cells of an individual. In certain instances, the efficiency of the cell uptake of the polymers or the micelles is enhanced by incorporation of targeting moieties into the polymers or micelles, or into or on the surface of the micelles. A “targeting moiety” (used interchangeably with “targeting agent”) recognizes the surface of a cell (e.g., a select cell). In some embodiments, targeting moieties recognize a cell surface antigen or bind to a receptor on the surface of the target cell. In certain other embodiments, the polymers or micelles described herein comprise a plurality of second monomeric units in addition to a plurality of targeting moiety-bearing monomeric units, wherein the second monomeric unit serves as a spacer unit affording groups of targeting moieties spatially positioned to maximize their binding to a multivalent receptor or target. Suitable targeting moieties include, by way of non-limiting example, antibodies, antibody-like molecules, or peptides, such as an integrin-binding peptides such as RGD-containing peptides, or small molecules, such as vitamins, e.g., folate, sugars such as lactose, galactose, N-acetyl galactose amine or other small molecules. Cell surface antigens include a cell surface molecule such as a protein, sugar, lipid or other antigen on the cell surface. In specific embodiments, the cell surface antigen undergoes internalization. Examples of cell surface antigens targeted by the targeting moieties of the micelles provided herein include, but are not limited, to the transferrin receptor type 1 and 2, the EGF receptor, HER2/Neu, VEGF receptors, integrins, NGF, CD2, CD3, CD4, CD8, CD19, CD2O, CD22, CD33, CD43, CD38, CD56, CD69, and the asialoglycoprotein receptor.

Targeting moieties (with or without spacer units) are attached, in various embodiments, to either end of a polymer (e.g., block copolymer) of the micelle, or to a side chain of a monomeric unit, or incorporated into a polymer block. Attachment of the targeting moiety to the polymer is achieved in any suitable manner, e.g., by any one of a number of conjugation chemistry approaches including but not limited to amine-carboxyl linkers, amine-sulfhydryl linkers, amine-carbohydrate linkers, amine-hydroxyl linkers, amine-amine linkers, carboxyl-sulfhydryl linkers, carboxyl-carbohydrate linkers, carboxyl-hydroxyl linkers, carboxyl-carboxyl linkers, sulfhydryl-carbohydrate linkers, sulfhydryl-hydroxyl linkers, sulfhydryl-sulfhydryl linkers, carbohydrate-hydroxyl linkers, carbohydrate-carbohydrate linkers, and hydroxyl-hydroxyl linkers. In specific embodiments, “click” chemistry is used to attach the targeting ligand to the block copolymers forming the micelles provided herein (for example of “click” reactions, see Wu, P.; Fokin, V. V. Catalytic Azide-Alkyne Cycloaddition: Reactivity and Applications. Aldrichim. Acta 2007, 40, 7-17). A large variety of conjugation chemistries are optionally utilized (see, for example, Bioconjugation, Aslam and Dent, Eds, Macmillan, 1998 and chapters therein, and Hermanson, G. T. (2008). Bioconjugate Techniques: 2^{nd} Edition. New York: Academic Press,). In some embodiments the linker is physiologically labile or contains a physiologically labile bond.

In some embodiments, targeting ligands are attached to a monomer and the resulting compound is then used in the polymerization synthesis of a polymer described herein (e.g., block copolymer) utilized in a micelle described herein. In certain embodiments, the monomers bearing such targeting ligands are vinylic monomers. In some instances, the monomers bearing such targeting ligands have the following Formula VII:

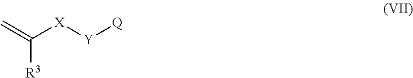

wherein:

X is selected from a group consisting of a covalent bond —C(O)O—, —C(O)NH—, a divalent C₅-C₁₀ aryl, and a divalent C₂-C₁₀ heteroaryl,

Y is a covalent bond or a linking group selected from optionally substituted divalent C₁-C₂₀ alkyl, optionally substituted divalent C₁-C₂₀ heteroalkyl, optionally substituted divalent C₁-C₂₀ alkenyl, optionally divalent substituted C₁-C₂₀ alkynyl, optionally substituted divalent C₃-C₂₀ cycloalkyl, optionally substituted divalent C₁-C₂₀ cycloheteroalkyl, optionally substituted divalent C₅-C₁₀ aryl, or optionally substituted divalent C₃-C₁₀ heteroaryl,

R³ is selected from the group consisting of hydrogen and optionally substituted C₁-C₄ alkyl,

Q is the targeting group selected from the group comprising a sugar residue, a peptide, a vitamin, or a small molecule (MW of less than 1.5 KDa) having an affinity for a cell-surface receptor or antigen.

In some embodiments, targeting moieties are attached to a block of a first block copolymer, or to a block of a second block copolymer in a mixed micelle. In some embodiments, the targeting ligand is attached to the sense or antisense strand of siRNA bound to a polymer or a polymer of the micelle. In certain embodiments, the targeting agent is attached to a 5′ or a 3′ end of the sense or the antisense strand.

In certain embodiments, polymers or micelles described herein comprise at least one targeting moiety at the alpha end of the polymer. In certain embodiments, targeting groups can be included at the alpha end of the polymer by the construction of a CTA (chain transfer agent) that incorporates the targeting group(s). In certain other embodiments, the incorporated targeting group(s) is tethered away from the reactive center of the CTA via a linker group, for example, a polyethylene glycol. In certain other embodiments, a CTA comprising a folic acid residue is used as the RAFT CTA for the preparation of polymers described herein. Alternatively, targeting groups can be included at the alpha end of the polymer by the modification of a chemical group on a macro-CTA with a moiety possessing a targeting group, for example by, but not limited to, the reaction of a carboxylic acid on the macro CTA with an alcohol possessing a targeting group.

In certain embodiments, polymers or micelles described herein comprise at least one targeting moiety at the omega end of the polymer. In certain embodiments, targeting groups can be included at the omega end of the polymer by synthesis of an additional polymer block, the additional block polymerized with a vinyl monomer possessing the targeting group. In certain other embodiments, targeting groups can be included at the omega end of the polymer by synthesis of an additional polymer block, the additional block polymerized with a vinyl monomer for attaching the targeting group. In certain other embodiments, targeting groups can be included at the omega end of the polymer by synthesis of an additional polymer block, the additional block polymerized with a vinyl monomer possessing the targeting group. In certain other embodiments, the polymerization is terminated by the addition of a maleimido monomer, wherein the maleimido monomer incorporates the targeting group(s). In certain other embodiments, the polymerization is terminated by the addition of a maleimido monomer, wherein the maleimido monomer incorporates functionality for attaching the targeting group(s) for example as described by Henry et al (End-Functionalized Polymers and Junction-Functionalized Diblock Copolymers Via RAFT Chain Extension with Maleimido Monomers. Scott M. Henry, Anthony J. Convertine, Danielle S. W. Benoit, Allan S. Hoffman and Patrick S. Stayton, Bioconjugate Chem., 2009, 20 (6), pp 1122-1128). In yet other embodiments, targeting groups can be included at the omega end of the polymer by synthetic modification of the Z group on the macro CTA, for example, reduction of a trithiocarbonyl group to a thiol group, followed to conjugation of the targeting group to the thiol on the polymer.

In certain embodiments, a micelle or polymer described herein comprises a mechanism for targeting the polymer, micelle, or therapeutic agent for a selected cell. In some embodiments, targeting of the polymer, micelle, or therapeutic agent is achieved by providing a copolymer described herein, or a micelle comprising such a copolymer, comprising a targeting moiety. In some embodiments, the targeting moiety is a ligand having affinity for one or more receptors effective for mediating endocytosis. In specific embodiments, the targeting moiety is covalently coupled to the first block.

Membrane Destabilization

In some embodiments, a polymer or micelle described herein (including portions, such as polymer subunits, thereof) is membrane-destabilizing at a pH of about 6.5 or lower, preferably at a pH ranging from about 5.0 to about 6.5, or at a pH of about 6.2 or lower, preferably at a pH ranging from about 5.0 to about 6.2, or at a pH of about 6.0 or lower, preferably at a pH ranging from about 5.0 to about 6.0. For example, in one embodiment, the polymer or micelle is membrane-destabilizing at a pH of or less than about 6.2, of or less than about 6.5, of or less than about 6.8, of or less than about 7.0. In certain embodiments, membrane destabilization is of any cellular membrane such as, by way of non-limiting example, an extracellular membrane, an intracellular membrane, a vesicle, an organelle, an endosome, a liposome, or a red blood cell. In some embodiments, membrane destabilizing polymers (e.g., copolymers) or membrane destabilizing block copolymers provided herein are membrane destabilizing (e.g., in an aqueous medium) at an endosomal pH.

In some embodiments, a polymer or micelle described herein (including portions, such as polymer subunits, thereof) is hemolytic at pH of or less than about 6.2, of or less than about 6.5, of or less than about 6.8, of or less than about 7.0. In further or alternative embodiments, the polymer or micelle (including portions, such as polymer subunits, thereof) is substantially non-hemolytic at pH greater than about 7.0. In specific embodiments, a polymer or micelle (including portions, such as polymer subunits, thereof) described herein is hemolytic at given concentration and a pH of about 6.2, and substantially non-hemolytic at the same concentration and at a pH greater than about 7.0. In more specific embodiments, the concentration is between 2 and 18 ug/mL, where there is 50-100% hemolysis at pH 5.8 and little or no significant hemolysis at pH 7.4. In certain embodiments, the hemolytic nature of a polymer or micelle (including portions, such as polymer subunits, thereof) described herein is determined in any suitable manner, e.g., by use of any standard hemolysis assay, such as an in vitro hemolysis assay.

In certain embodiments, a polymer or micelle described herein (including portions, such as polymer subunits, thereof) is endosome disruptive. In some embodiments, a polymer or micelle described herein (including portions, such as polymer subunits, thereof) is endosome disruptive at pH of or less than about 6.2, of or less than about 6.5, of or less than about 6.8, of or less than about 7.0. In certain embodiments, a polymer In certain embodiments, the endosome disruptive nature of a polymer or micelle (including portions, such as polymer subunits, thereof) described herein is determined in any suitable manner, e.g., by use of any standard hemolysis assay, such as an in vitro endosomolysis assay, or an in vivo non-human mammal endosomolysis assay.

Therapeutic Agents

Provided in certain embodiments is a polymer or micelle, as described herein in combination with a therapeutic agent. In specific embodiments, the therapeutic agent is a polynucleotide (e.g., oligonucleotide) or a peptide. The therapeutic agent may be used prophylactically, as a vaccine or to treat a medical condition.

In various embodiments, research reagents, diagnostic agents, and/or therapeutic agents are attached to the block copolymer or a micelle containing the block copolymers in any suitable manner. In specific embodiments, attachment is achieved through covalent bonds, non-covalent interactions, static interactions, hydrophobic interactions, or the like, or combinations thereof. In some embodiments, the research reagents, diagnostic agents, and/or therapeutic agents are attached to an intermediate or second block of block copolymers, or micelles thereof. In certain embodiments, the research reagents, diagnostic agents, or therapeutic agents form the intermediate or second block of a block copolymer, or micelle thereof. In some embodiments, the research reagents, diagnostic agents, or therapeutic agents are in the shell of the micelle.

In some embodiments, provided herein is a micelle comprising a first therapeutic agent in the shell of the micelle and a second therapeutic agent in the core of the micelle. In specific embodiments, the first therapeutic agent is a polynucleotide. And the second therapeutic agent is a hydrophobic drug. In certain embodiments, provided herein is a micelle comprising a hydrophobic drug (e.g., small molecule hydrophobic drug) in the core of the micelle.

In certain embodiments, provided herein is a micelle comprising at least 1-5, 5-250, 5-1000, 250-1000, at least 2, at least 5, at least 10, at least 20, or at least 50 polymers with attached therapeutic agents. In some embodiments, provided herein is a composition comprising a plurality of micelles described herein, wherein the micelles therein comprise, on average, at least 1-5, 5-250, 5-1000, 250-1000, at least 2, at least 5, at least 10, at least 20, or at least 50 polymers with attached therapeutic agents.

In some embodiments, therapeutic agents, diagnostic agents, etc., are selected from, by way of non-limiting example, at least one nucleotide (e.g., a polynucleotide), at least one carbohydrate or at least one amino acid (e.g., a peptide). In specific embodiments, the therapeutic agent is a polynucleotide, an oligonucleotide, a gene expression modulator, a knockdown agent, an sRNA, an RNAi agent, a dicer substrate, an miRNA, an shRNA, an antisense oligonucleotide, or an aptamer. In other specific embodiments, the therapeutic agent is an aiRNA (Asymmetric RNA duplexes mediate RNA interference in mammalian cells. Xiangao Sun, Harry A Rogoff, Chiang J Li Nature Biotechnology 26, 1379-1382 (2008)). In certain embodiments, the therapeutic agent is a protein, peptide, enzyme, antibody, or antibody fragment. In some embodiments, the therapeutic agent is a carbohydrate, or a small molecule with a molecular weight of greater than about 500 Daltons.

In some embodiments, a polynucleotide associated with a polymer or micelle described herein is an oligonucleotide gene expression modulator. In certain embodiments, the polynucleotide is an oligonucleotide aptamer. In some embodiments, the polynucleotide is an oligonucleotide knockdown agent. In certain embodiments, the polynucleotide is an interfering RNA. In some embodiments, the polynucleotide is an oligonucleotide selected from an sRNA, an antisense oligonucleotide, a dicer substrate, an miRNA, an aiRNA or an shRNA. In specific embodiments, the polynucleotide is a sRNA.

In some embodiments, a therapeutic agent (e.g., oligonucleotide) is chemically conjugated to the polymer or to the micelle and/or to one or more polymer of the micelle by any suitable chemical conjugation technique. In some embodiments, micelles containing an RNAi agent are formed by conjugation of the RNAi agent with an already formed micelle comprising a plurality of polymers (e.g., block copolymers). In other embodiments, micelles containing an RNAi agent are formed by conjugation of the RNAi agent with a polymer (e.g., a membrane destabilizing block copolymer) and subsequently forming the micelle in any suitable manner, e.g., by self assembly of the resulting conjugates into a micelle comprising the RNAi agent. In various embodiments, such a micelle optionally further comprises unconjugated polymers (e.g., block copolymers) that are similar, identical, or different than those conjugated to the RNAi agent. The covalent bond between a polymer and a therapeutic agent of a micelle described herein is, optionally, non-cleavable, or cleavable. In certain embodiments, a precursor of one or more RNAi agent (e.g. a dicer substrate) is attached to the micelle or to the polymeric units of the micelle by a non-cleavable bond. In some embodiments, one or more RNAi agent is attached through a cleavable bond. In certain embodiments, the cleavable bonds utilized in the micelles described herein include, by way of non-limiting example, disulfide bonds (e.g., disulfide bonds that dissociate in the reducing environment of the cytoplasm). In some embodiments, covalent association between a micelle (including the components thereof) and a therapeutic agent (e.g., an oligonucleotide or sRNA) is achieved through any suitable chemical conjugation method, including but not limited to amine-carboxyl linkers, amine-sulfhydryl linkers, amine-carbohydrate linkers, amine-hydroxyl linkers, amine-amine linkers, carboxyl-sulfhydryl linkers, carboxyl-carbohydrate linkers, carboxyl-hydroxyl linkers, carboxyl-carboxyl linkers, sulfhydryl-carbohydrate linkers, sulfhydryl-hydroxyl linkers, sulfhydryl-sulfhydryl linkers, carbohydrate-hydroxyl linkers, carbohydrate-carbohydrate linkers, and hydroxyl-hydroxyl linkers. In some embodiments, conjugation is also performed with pH-sensitive bonds and linkers, including, but not limited to, hydrazone and acetal linkages. Any other suitable conjugation method is optionally utilized as well, for example a large variety of conjugation chemistries are available (see, for example, Bioconjugation, Aslam and Dent, Eds, Macmillan, 1998 and chapters therein).

In specific embodiments, the agent delivered by the means of the polymer or the micelle provided herein is a diagnostic agent. In some embodiments, the diagnostic agent is a diagnostic imaging agent, e.g., an agent useful in imaging the mammalian vascular system which includes but is not limited to position emission tomography (PET) agents, computerized tomography (CT) agents, magnetic resonance imaging (MRI) agents, nuclear magnetic imaging agents (NMI), fluoroscopy agents and ultrasound contrast agents. Such diagnostic agents include radioisotopes of such elements as iodine (I), including ¹²³I, ¹²⁵I, ¹³¹I, etc., barium (Ba), gadolinium (Gd), technetium (Tc), including ⁹⁹Tc, phosphorus (P), including ³¹P, iron (Fe), manganese (Mn), thallium (Tl), chromium (Cr), including ⁵¹Cr, carbon (C), including ¹⁴C, or the like, fluorescently labeled compounds, or their complexes, chelates, adducts and conjugates. In other embodiments, the diagnostic agent is a marker gene that encode proteins that are readily detectable when expressed in a cell (including, but not limited to, β-galactosidase, green fluorescent protein, luciferase, and the like) and labeled nucleic acid probes (e.g., radiolabeled or fluorescently labeled probes). In some embodiments, covalent conjugation of diagnostics agents to the polymers or the micelles provided herein is achieved according to a variety of conjugation processes. In some embodiments, a radiolabeled monomer (e.g., a ¹⁴C-labeled monomer) is incorporated into the polymeric backbone of the micelle. In some embodiments, a polymer or a micelle associated with a diagnostic agent comprises a targeting moiety.

Method for Preparing Micelle Composition

In a preferred embodiment, a micelle comprising a polymer of the present invention may be prepared by in a process comprising the steps of (i) dissolving a polymer having a hydrophobic block in a water miscible solvent, (ii) dissolving a polynucleotide in an aqueous solution and (iii) combining the two to conjugate the polynucleotide to the hydrophobic block of the polymer and form the micelle.

Pharmaceutical Compositions

The compositions comprising a polymer or a polymeric micelle and an agent, such as a biomolecular agent (e.g., a polynucleotide), can be a pharmaceutical composition. Such pharmaceutical composition can comprise, for example, a polymer or a polymeric micelle, a biomolecular agent, such as a polynucleotide, and a pharmaceutically acceptable excipient.

Polymer and micelle compositions provided herein (e.g., those attached to one or more RNAi agent therapeutic agent, such as one or more oligonucleotide) are optionally provided in a composition (e.g., pharmaceutically acceptable composition). In some embodiments, a polymer or micelle composition provided herein is administered to a patient in any suitable manner, e.g., with or without stabilizers, buffers, and the like, to form a pharmaceutical composition. In some embodiments, the polymer or micelle composition provided herein is formulated and used as tablets, capsules or elixirs for oral administration, suppositories for rectal administration, sterile solutions, suspensions or solutions for injectable administration, and any other suitable compositions.

Provided are pharmaceutically acceptable formulations of a polymer or micelle composition comprising at least one RNAi therapeutic agent described herein. These formulations include salts of the above compounds, e.g., acid addition salts, e.g., salts of hydrochloric, hydrobromic, acetic acid, and benzene sulfonic acid. A pharmacological composition or formulation refers to a composition or formulation in a form suitable for administration, e.g., systemic administration, into a cell or patient, including for example a human. Suitable forms, in part, depend upon the use or the route of entry, e.g., oral, transdermal, or by injection. Thus, in specific embodiments wherein the polymer or micelle composition comprises and is delivering a polynucleotide, the formulation is in a form that does not prevent the polymer or micelle composition and, more specifically, the polynucleotide (e.g., oligonucleotide or siRNA) from reaching a target cell with the polynucleotide intact and/or functional. For example, in certain embodiments, pharmacological compositions injected into the blood stream are soluble and/or dispersible. Moreover, pharmaceutical compositions described herein are, preferably, non-toxic. In some embodiments, wherein a polymer or micelle composition provided herein is administered for therapeutic benefit, a therapeutic effective amount of the composition comprising an RNAi therapeutic agent (e.g., a polynucleotide, such as an siRNA) is administered. In an exemplary embodiment, a therapeutically effective amount includes a sufficient amount of a polymer or micelle composition provided herein to provide about 10 mg or less of siRNA per kg of individual.

In some embodiments, pharmaceutical compositions comprising a polymer or micelle composition, which comprise an RNAi therapeutic agent (e.g., a polynucleotide, such as an siRNA), are administered systemically. As used herein, “systemic administration” means in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular. In some embodiments, the compositions are administered topically.

In some embodiments, the compositions are prepared for storage or administration and include a pharmaceutically effective amount of the therapeutic agent comprising a polymer or micelle composition provided herein in a pharmaceutically acceptable carrier or diluent. Any acceptable carriers or diluents are optionally utilized herein. Specific carriers and diluents and are described, e.g., in Remington's Pharmaceutical Sciences, Mack Publishing Co., A. R. Gennaro Ed., 1985. For example, preservatives, stabilizers, dyes and flavoring agents are optionally added. These include sodium benzoate, sorbic acid and esters of p-hydroxybenzoic acid. In addition, antioxidants and suspending agents are optionally used. As used herein, the term “pharmaceutically acceptable carrier” means a non-toxic, inert solid, semi-solid or liquid filler, diluent, encapsulating material or formulation auxiliary of any type. Some examples of materials optionally used as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. In some embodiments, the pharmaceutical compositions provided herein are administered to humans and/or to animals, orally, rectally, parenterally, intracistemally, intravaginally, intranasally, intraperitoneally, topically (as by powders, creams, ointments, or drops), bucally, or as an oral or nasal spray.

In various embodiments, liquid dosage forms for oral administration include pharmaceutically acceptable emulsions, microemulsions, solutions, suspensions, syrups, and elixirs. In addition to the active ingredients (i.e., micelle-oligonucleotide complexes provided herein), the liquid dosage forms optionally further contain inert diluents or excipients, such as by way of non-limiting example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions optionally also include adjuvants such as wetting agents, emulsifying and suspending agents, sweetening, flavoring, and perfuming agents.

In some embodiments, injectable preparations, for example, sterile injectable aqueous or oleaginous suspensions are formulated according in any suitable manner, e.g., using dispersing agents, wetting agents and/or suspending agents. The sterile injectable preparation is, optionally, a sterile injectable solution, suspension, or emulsion in a nontoxic parenterally acceptable diluent or solvent, for example, as a solution in 1,3-butanediol. Among the acceptable vehicles and solvents that are optionally employed are water, Ringer's solution, U.S.P. and isotonic sodium chloride solution. In addition, sterile, fixed oils are conventionally employed as a solvent or suspending medium. For this purpose any bland fixed oil is optionally employed including synthetic mono- or diglycerides. In additional embodiments, fatty acids such as oleic acid are used in the preparation of injectables. In a specific embodiment, the polymer or micelle composition provided herein is solubilized in a carrier fluid comprising 1% (w/v) sodium carboxymethyl cellulose and 0.1% (v/v) Tween 80.

In some embodiments, the injectable formulations are sterilized, for example, by filtration through a bacteria-retaining filter, or by incorporating sterilizing agents in the form of sterile solid compositions which are optionally dissolved or dispersed in sterile water or other sterile injectable medium prior to use.

In certain embodiments, compositions for rectal or vaginal administration are suppositories. Suppositories are optionally prepared by mixing the therapeutic agent comprising a polymer or micelle composition provided herein with suitable non-irritating excipients or carriers such as cocoa butter, polyethylene glycol, or a suppository wax which are solid at ambient temperature but liquid at body temperature and therefore melt in the rectum or vaginal cavity and release a polymer or micelle composition provided herein.

Suitable solid dosage forms for oral administration include, by way of non-limiting example, capsules, tablets, pills, powders, and granules. In such solid dosage forms, a polymer or micelle composition provided herein comprising an RNAi therapeutic agent (e.g., oligonucleotide) are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type are also optionally employed as fillers in soft and hard-filled gelatin capsules using such excipients as lactose or milk sugar as well as high molecular weight polyethylene glycols and the like.

In some embodiments, the solid dosage forms of tablets, dragees, capsules, pills, and granules are prepared with coatings and shells such as enteric coatings and other suitable coatings. They optionally contain opacifying agents. In certain embodiments, they are of a composition that they release the active ingredient(s) only, or preferentially, in a certain part of the intestinal tract, optionally, in a delayed manner. Examples of suitable embedding compositions include, by way of non-limiting example, polymeric substances and waxes.

Solid compositions of a similar type are optionally employed as fillers in soft and hard-filled gelatin capsules using such excipients as lactose or milk sugar as well as high molecular weight polyethylene glycols and the like.

Dosage forms for topical or transdermal administration of an inventive pharmaceutical composition include, by way of non-limiting example, ointments, pastes, creams, lotions, gels, powders, solutions, sprays, inhalants, or patches. In some embodiments, therapeutic agents comprising a polymer or micelle composition provided herein are admixed under sterile conditions with a pharmaceutically acceptable carrier and, optionally, one or more preservative, one or more buffer, or a combination thereof (e.g., as may be required). Ophthalmic formulation, ear drops, and eye drops are also contemplated as being within the scope of this invention.

Ointments, pastes, creams, and gels provided herein optionally contain, in addition to the polymer or micelle composition provided herein, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays optionally contain, in addition to a polymer or micelle composition provided herein, excipients such as lactose, talc, silicic acid, aluminum hydroxide, calcium silicates, and polyamide powder, or mixtures of these substances. In some embodiments, sprays additionally contain customary propellants such as chlorofluorohydrocarbons.

Transdermal patches have the added advantage of providing controlled delivery of a compound to the body. Such dosage forms are made in any suitable manner, e.g., by dissolving or dispensing the microparticles or nanoparticles in a proper medium. Absorption enhancers are optionally used to increase the flux of the compound across the skin. The rate can be controlled by either providing a rate controlling membrane or by dispersing a polymer or micelle composition provided herein in a polymer matrix or gel.

In some aspects of the invention, a polymer or micelle composition provides some properties (e.g. mechanical, thermal, etc.) that are usually performed by excipients, thus decreasing the amount of such excipients required for the formulation.

Therapeutic Uses

Compositions comprising polymers or polymeric micelles and an agent such as a polynucleotide can be used in various methods.

Generally, such compositions can be used for example in a method for intracellular delivery of an agent such as a polynucleotide. The composition comprising a polymer or a polymeric micelle and an agent (e.g., a polynucleotide) associated therewith can be exposed to and contacted with a with a cell surface (e.g., via directed targeting) in a medium at a first pH. The composition is introduced into an endosomal membrane within the cell, for example, through endocytosis and, in some embodiments, through receptor mediated endocytosis. The endosomal membrane is destabilized (e.g., by a constituent polymer or block thereof, which is a membrane destabilizing polymer), thereby delivering the composition or the agent (e.g., polynucleotide) to the cytosol of the cell. The medium can be an in vitro medium. The medium can be an in vitro medium such as a physiological medium.

Generally, for example, such compositions can be used for modulating the activity of an intracellular target in a cell. The agent, such as a polynucleotide, can be delivered to the cytosol of a cell according to the method described in the immediately preceding paragraph. The agent (e.g., polynucleotide) is allowed to interact with the intracellular target, thereby modulating the activity of the intracellular target.

More specifically, for example, in some embodiments, the compositions comprising polymers or polymeric micelles (e.g., micelles) provided herein are useful in treating a subject at risk for or afflicted with disorders associated with and/or caused by high plasma levels or cholesterol, apolipoprotein b, and/or LDL cholesterol, e.g., hypercholesterolemia. In certain embodiments, the treatment comprises providing a polymeric micelle and a therapeutic agent (e.g., an oligonucleotide agent) associated therewith, wherein the therapeutic agent silences (e.g., by cleavage) a gene, or a gene product that promotes such condition. In some embodiments the therapeutic agent (e.g., an oligonucleotide or RNAi agent) silences proprotein convertase subtilisin/kexin type 9 (PCSK9) gene responsible for regulation of low density lipoprotein (LDLR) levels and function and, thus, polymers or polymeric micelles comprising such therapeutic agents are used to treat a subject having or at risk for a disorder characterized by unwanted PCSK9 expression, e.g., disorders associated with and/or caused by high plasma levels or cholesterol, apolipoprotein b, and/or LDL cholesterol, e.g., hypercholesterolemia. In some embodiments, the polymers or polymeric micelles deliver a PCSK9 silencing polynucleotide agent (e.g., siRNA) to a cell expressing PCSK9. In some embodiments, the cell is a liver cell.

In some embodiments, the polymers or polymeric micelles (e.g., micelles) provided herein are useful in treating a subject at risk for or afflicted with unwanted cell proliferation (e.g., malignant or nonmalignant cell proliferation). The treatment comprises providing a composition comprising a polymer or a polymeric micelle and a therapeutic agent (e.g., an oligonucleotide agent), wherein the therapeutic agent can silence (e.g., by cleavage) a gene or a gene product that promotes unwanted cell proliferation; and administering a therapeutically effective dose of the polymer or polymeric micelle to a subject (e.g., a human subject.). In some embodiments, the therapeutic agent is a polynucleotide (e.g., an oligonucleotide) that is homologous to and can silence (e.g., by cleavage) a gene.

In certain embodiments, the gene is, but is not limited to, a growth factor or growth factor receptor gene, a phosphatase, a kinase, e.g., a protein tyrosine, serine, or threonine kinase gene, an adaptor protein gene, a gene encoding a G protein superfamily molecule, or a gene encoding a transcription factor. In some instances, the composition comprises a polymer or a polymeric micelle and a polynucleotide that silences a gene that is expressed in a specific tissue or organ including, but not limited to, lung, pancreas, liver, kidney, ovary, muscle, skin, breast, colon, stomach, and the like.

In some embodiments, the oligonucleotide agent silences one or more of the following genes the PDGF beta gene, and thus can be used to treat a subject having or at risk for a disorder characterized by unwanted PDGF beta expression, e.g., testicular and lung cancers; an Erb-B gene (e.g., Erb-B-2 or Erb-B-3), and thus can be used to treat a subject having or at risk for a disorder characterized by unwanted Erb-B expression, e.g., breast or lung cancer; the Src gene, and thus can be used to treat a subject having or at risk for a disorder characterized by unwanted Src expression, e.g., colon cancers; the CRK gene, and thus can be used to treat a subject having or at risk for a disorder characterized by unwanted CRK expression, e.g., colon and lung cancers; the GRB2 gene, and thus can be used to treat a subject having or at risk for a disorder characterized by unwanted GRB2 expression, e.g., squamous cell carcinoma; the RAS gene, and thus can be used to treat a subject having or at risk for a disorder characterized by unwanted RAS expression, e.g., pancreatic, colon and lung cancers and chronic leukemia; the MEKK gene, and thus can be used to treat a subject having or at risk for a disorder characterized by unwanted MEKK expression, e.g., squamous cell carcinoma, melanoma, or leukemia; the JNK gene, and thus can be used to treat a subject having or at risk for a disorder characterized by unwanted JNK expression, e.g., pancreatic or breast cancers; the RAF gene, and thus can be used to treat a subject having or at risk for a disorder characterized by unwanted RAF expression, e.g., lung cancer or leukemia; the Erk1/2 gene, and thus can be used to treat a subject having or at risk for a disorder characterized by unwanted Erk1/2 expression, e.g., lung cancer; the PCNA(p21) gene, and thus can be used to treat a subject having or at risk for a disorder characterized by unwanted PCNA expression, e.g., lung cancer; the MYB gene, and thus can be used to treat a subject having or at risk for a disorder characterized by unwanted MYB expression, e.g., colon cancer or chronic myelogenous leukemia; the c-MYC gene, and thus can be used to treat a subject having or at risk for a disorder characterized by unwanted c MYC expression, e.g., Burkitt's lymphoma or neuroblastoma; the JUN gene, and thus can be used to treat a subject having or at risk for a disorder characterized by unwanted JUN expression, e.g., ovarian, prostate or breast cancers; the FOS gene, and thus can be used to treat a subject having or at risk for a disorder characterized by unwanted FOS expression, e.g., skin or prostate cancers; the BCL 2 gene, and thus can be used to treat a subject having or at risk for a disorder characterized by unwanted BCL-2 expression, e.g., lung or prostate cancers or Non Hodgkin lymphoma; the Cyclin D gene, and thus can be used to treat a subject having or at risk for a disorder characterized by unwanted Cyclin D expression, e.g., esophageal and colon cancers; the VEGF gene, and thus can be used to treat a subject having or at risk for a disorder characterized by unwanted VEGF expression, e.g., esophageal and colon cancers; the EGFR gene, and thus can be used to treat a subject having or at risk for a disorder characterized by unwanted EGFR expression, e.g., breast cancer; the Cyclin A gene, and thus can be used to treat a subject having or at risk for a disorder characterized by unwanted Cyclin A expression, e.g., lung and cervical cancers; the Cyclin E gene, and thus can be used to treat a subject having or at risk for a disorder characterized by unwanted Cyclin E expression, e.g., lung and breast cancers; the WNT-1 gene, and thus can be used to treat a subject having or at risk for a disorder characterized by unwanted WNT 1 expression, e.g., basal cell carcinoma; the beta catenin gene, and thus can be used to treat a subject having or at risk for a disorder characterized by unwanted beta catenin expression, e.g., adenocarcinoma or hepatocellular carcinoma; the c-MET gene, and thus can be used to treat a subject having or at risk for a disorder characterized by unwanted c-MET expression, e.g., hepatocellular carcinoma; the PKC gene, and thus can be used to treat a subject having or at risk for a disorder characterized by unwanted PKC expression, e.g., breast cancer; the NFKB gene, and thus can be used to treat a subject having or at risk for a disorder characterized by unwanted NFKB expression, e.g., breast cancer; the STAT3 gene, and thus can be used to treat a subject having or at risk for a disorder characterized by unwanted STAT3 expression, e.g., prostate cancer; the survivin gene, and thus can be used to treat a subject having or at risk for a disorder characterized by unwanted survivin expression, e.g., cervical or pancreatic cancers; the Her2/Neu gene, and thus can be used to treat a subject having or at risk for a disorder characterized by unwanted Her2/Neu expression, e.g., breast cancer; the Topoisomerase I gene, and thus can be used to treat a subject having or at risk for a disorder characterized by unwanted Topoisomerase I expression, e.g., ovarian and colon cancers; the Topoisomerase II alpha gene, and thus can be used to treat a subject having or at risk for a disorder characterized by unwanted Topoisomerase II expression, e.g., breast and colon cancers.

In other embodiments the oligonucleotide agent silences mutations in one of the following genes: the p73 gene, and thus can be used to treat a subject having or at risk for a disorder characterized by unwanted p73 expression, e.g., colorectal adenocarcinoma; the p21(WAF1/CIP1) gene, and thus can be used to treat a subject having or at risk for a disorder characterized by unwanted p21(WAF1/CIP1) expression, e.g., liver cancer; the p27(KIP1) gene, and thus can be used to treat a subject having or at risk for a disorder characterized by unwanted p27(KIP1) expression, e.g., liver cancer; the PPM1 D gene, and thus can be used to treat a subject having or at risk for a disorder characterized by unwanted PPM1 D expression, e.g., breast cancer; the RAS gene, and thus can be used to treat a subject having or at risk for a disorder characterized by unwanted RAS expression, e.g., breast cancer; the caveolin I gene, and thus can be used to treat a subject having or at risk for a disorder characterized by unwanted caveolin I expression, e.g., esophageal squamous cell carcinoma; the MIB I gene, and thus can be used to treat a subject having or at risk for a disorder characterized by unwanted MIB I expression, e.g., male breast carcinoma (MBC); MTAI gene, and thus can be used to treat a subject having or at risk for a disorder characterized by unwanted MTAI expression, e.g., ovarian carcinoma; the M68 gene, and thus can be used to treat a subject having or at risk for a disorder characterized by unwanted M68 expression, e.g., human adenocarcinomas of the esophagus, stomach, colon, and rectum.

In some embodiments the oligonucleotide agent silences mutations in tumor suppressor genes and thus can be used as a method to promote apoptotic activity in combination with chemotherapeutics. In some embodiments the in the tumor suppressor gene is selected from one or more of the following tumor suppressor genes: the p53 tumor suppressor gene, the p53 family member DN p63, the pRb tumor suppressor gene, the APC1 tumor suppressor gene, the BRCA1 tumor suppressor gene, the PTEN tumor suppressor gene.

In some embodiments the oligonucleotide agent silences one of the following fusion genes: mLL fusion genes, e.g., mLL AF9, and thus can be used to treat a subject having or at risk for a disorder characterized by unwanted mLL fusion gene expression, e.g., acute leukemias; the BCR/ABL fusion gene, and thus can be used to treat a subject having or at risk for a disorder characterized by unwanted BCR/ABL fusion gene expression, e.g., acute and chronic leukemias; the TEL/AML1 fusion gene, and thus can be used to treat a subject having or at risk for a disorder characterized by unwanted TEL/AML1 fusion gene expression, e.g., childhood acute leukemia; the EWS/FLI1 fusion gene, and thus can be used to treat a subject having or at risk for a disorder characterized by unwanted EWS/FLI1 fusion gene expression, e.g., Ewing Sarcoma; the TLS/fusion gene, and thus can be used to treat a subject having or at risk for a disorder characterized by unwanted TLS/FUS1 fusion gene expression, e.g., Myxoid liposarcoma; the PAX3/FKHR fusion gene, and thus can be used to treat a subject having or at risk for a disorder characterized by unwanted PAX3/FKHR fusion gene expression, e.g., Myxoid liposarcoma; the AML1/ETO fusion gene, and thus can be used to treat a subject having or at risk for a disorder characterized by unwanted AML1/ETO fusion gene expression, e.g., acute leukemia.

In some aspects herein the compositions comprising the polymers or polymeric micelles and an agent, such as a polynucleotide, provide therapeutic agents for treating a subject, e.g., a human, at risk for or afflicted with a disease or disorder that may benefit by angiogenesis inhibition e.g., cancer or retinal degeneration. The treatment comprises providing a polymer or a polymeric micelle comprising an oligonucleotide agent, wherein said oligonucleotide agent is homologous to and/or can silence, e.g., by cleavage, a gene that mediates angiogenesis (e.g., VEGF R1, VEGF R2, or gene encoding signaling proteins for these receptors' pathways); and administering a therapeutically effective dosage of said polymer or polymeric micelle comprising the oligonucleotide agent to a subject, e.g., a human subject.

In some embodiments the oligonucleotide agent silences one of the following genes: the alpha v integrin gene, and thus can be used to treat a subject having or at risk for a disorder characterized by unwanted alpha V integrin, e.g., brain tumors or tumors of epithelial origin; the Flt-1 receptor gene, and thus can be used to treat a subject having or at risk for a disorder characterized by unwanted Flt-1 receptors, e.g., cancer and rheumatoid arthritis; the tubulin gene, and thus can be used to treat a subject having or at risk for a disorder characterized by unwanted tubulin, e.g., cancer and retinal neovascularization.

In some aspects the composition comprising polymers pr polymeric micelles and an oligonucleotide agent relate to a method of treating a subject infected with a virus or at risk for or afflicted with a disorder or disease associated with a viral infection. The method comprises providing a polymer or a polymeric micelle comprising an oligonucleotide agent, wherein said oligonucleotide agent is homologous to and/or can silence, e.g., by cleavage, a viral gene or a cellular gene that mediates viral function, e.g., entry or growth; and administering a therapeutically effective dose of said oligonucleotide agent to a subject, e.g., a human subject.

In some embodiments, the composition comprising polymers or polymeric micelles and an oligonucleotide agent are useful in treatment of subjects infected with the Human Papilloma Virus (HPV) or at risk for or afflicted with a disorder mediated by HPV, e.g., cervical cancer.

In some embodiments, a composition comprising a polymer or a polymeric micelle and an oligonucleotide agent silencing expression of a HPV gene is reduced. In some embodiments, the HPV gene is selected from the group of E2, E6, or E7.

In another embodiment the expression of a human gene that is required for HPV replication is reduced.

In some embodiments, the composition comprises a polymer or a polymeric micelle and an oligonucleotide agent useful in treating patients infected by the Human Immunodeficiency Virus (HIV) or at risk for or afflicted with a disorder mediated by HIV, e.g., Acquired Immune Deficiency Syndrome (AIDS). In some embodiments, the expression of an HIV gene is reduced. In other embodiments, the HIV gene is CCR5, Gag, or Rev. In some embodiments the expression of a human gene that is required for HIV replication is reduced. In some embodiments, the gene is CD4 or Tsg101.

In some embodiments, the composition comprises a polymer or a polymeric micelle and an oligonucleotide agent useful for treating patients infected by the Hepatitis B Virus (HBV) or at risk for or afflicted with a disorder mediated by HBV, e.g., cirrhosis and heptocellular carcinoma. In one embodiment, the expression of a HBV gene is reduced. In another embodiment, the targeted HBV gene encodes one of the groups of the tail region of the HBV core protein, the pre cregious (pre c) region, or the cregious (c) region. In other embodiments, a targeted HBV RNA sequence is comprised of the poly(A) tail. In some embodiments, the expression of a human gene that is required for HBV replication is reduced.

In some embodiments, the composition comprises a polymer or a polymeric micelle and an oligonucleotide agent useful for treating patients infected with or at risk for or afflicted with a disorder mediated by a virus selected from the following viruses: the Hepatitis A Virus (HAV); Hepatitis C Virus (HCV); any of the group of Hepatitis Viral strains comprising Hepatitis D, E, F, G, or H; the Respiratory Syncytial Virus (RSV); the herpes Cytomegalovirus (CMV); the herpes Epstein Barr Virus (EBV); Kaposi's Sarcoma associated Herpes Virus (KSHV); the JC Virus (JCV); myxovirus (e.g., virus causing influenza), rhinovirus (e.g., virus causing the common cold), or coronavirus (e.g., virus causing the common cold); the St. Louis Encephalitis flavivirus; the Tick borne encephalitis flavivirus; the Murray Valley encephalitis flavivirus; the dengue flavivirus; the Simian Virus 40 (SV40); the encephalomyocarditis virus (EMCV); the measles virus (MV); the Varicella zoster virus (VZV); an adenovirus (e.g., virus causing a respiratory tract infection); the poliovirus; or a poxvirus (a poxvirus causing smallpox). In some embodiments, the expression of a human gene that is required for the replication of these viruses is reduced.

In some embodiments, the composition comprises a polymer or a polymeric micelle and an oligonucleotide agent useful for treating patients infected by the Herpes Simplex Virus (HSV) or at risk for or afflicted with a disorder mediated by HSV, e.g., genital herpes and cold sores, as well as life threatening or sight impairing disease, e.g., mainly in immunocompromised patients. In some embodiments, the expression of a HSV gene is reduced. In other embodiments, the targeted HSV gene encodes DNA polymerase or the helicase primase. In some embodiments the expression of a human gene that is required for HSV replication is reduced.

In some embodiments, the composition comprises a polymer or a polymeric micelle and an oligonucleotide agent useful for treating patients infected by the West Nile Virusor at risk for or afflicted with a disorder mediated by West Nile Virus. In some embodiments, the expression of a West Nile Virus gene is reduced. In other preferred embodiments, the West Nile Virus gene is selected from the group comprising E, NS3, or NS5. In some embodiments, the expression of a human gene that is required for West Nile Virus replication is reduced.

In some embodiments, the polymer or polymeric micelle comprises an oligonucleotide agent useful for treating patients infected by the Human T Cell Lymphotropic Virus (HTLV) or a disease or disorder associated with this virus, e.g., leukemia or myelopathy. In some embodiments, the expression of a HTLV gene is reduced. In some embodiments, the HTLV1 gene is the Tax transcriptional activator. In some embodiments, the expression of a human gene that is required for HTLV replication is reduced.

In some aspects, the composition comprises a polymer or a polymeric micelle and an oligonucleotide agent useful for treating a subject infected with a pathogen, e.g., a bacterial, amoebic, parasitic, or fungal pathogen. The method of treatment comprises providing a polymer or a polymeric micelle comprising an oligonucleotide agent, wherein said oligonucleotide is homologous to and/or can silence, e.g., by cleavage of a pathogen gene or a gene involved in the pathogen's growth; and administering a therapeutically effective dose of said oligonucleotide agent to a subject, e.g., a human subject. The target gene can be selected from a gene involved in the pathogen's growth, cell wall synthesis, protein synthesis, transcription, energy metabolism, e.g., the Krebs cycle, or toxin production.

Thus, in some embodiments, the composition comprises a polymer or a polymeric micelle and an oligonucleotide agent useful for of treating patients infected by a plasmodium that causes malaria. In some embodiments, the expression of a plasmodium gene is reduced. In other embodiments, the gene is apical membrane antigen 1 (AMA1). In some embodiments, the expression of a human gene that is required for plasmodium replication is reduced.

In some embodiments, the polymer or polymeric micelle comprises an oligonucleotide agent useful for treating patients infected by Mycobacterium ulcerans, Mycobacterium tuberculosis, Mycobacterium leprae, Staphylococcus aureus, Streptococcus pneumoniae, Streptococcus pyogenes, Chlamydia pneumoniae, Mycoplasma pneumoniae, or a disease or disorder associated with any of these pathogens. In some embodiments, the expression of a bacterial gene and/or a human gene that is required for the replication of these bacteria is reduced.

In some embodiments, the diseases treated by the compositions comprising a polymer or a polymeric micelle and an agent as provided herein may be systemic or present in a specific tissue, e.g., the lung, skin, liver, breast, kidney, pancreas, CNS, or the like. In certain aspects, the oligonucleotide silences a gene that mediates or is involved in a metabolic disease or disorder, e.g., diabetes, obesity, and the like. In certain embodiments, the oligonucleotide silences a gene that mediates or is involved in a pulmonary disease or disorder, e.g., chronic obstructive pulmonary disease (COPD), cystic fibrosis, or lung cancer. In some aspects herein, the polymers or polymeric micelles comprise an oligonucleotide agent useful for and/or related to a method of treating a subject, e.g., a human, at risk for or afflicted with a disease or disorder characterized by an unwanted immune response, e.g., an inflammatory disease or disorder or an autoimmune disease or disorder. The method comprises providing a polymer or a polymeric micelle comprising an oligonucleotide agent, wherein said oligonucleotide agent is homologous to and/or can silence, e.g., by cleavage, a gene that mediates an unwanted immune response; and administering said oligonucleotide agent to a subject, e.g., a human subject. In some embodiments, the disease or disorder is an ischemia or reperfusion injury, e.g., ischemia or reperfusion injury associated with acute myocardial infarction, unstable angina, cardiopulmonary bypass, surgical intervention, e.g., angioplasty, e.g., percutaneous transluminal coronary angioplasty, the response to a transplanted organ or tissue, e.g., transplanted cardiac or vascular tissue; or thrombolysis. In other embodiments, the disease or disorder is restenosis, e.g., restenosis associated with surgical intervention, e.g., angioplasty, e.g., percutaneous transluminal coronary angioplasty. In other embodiments, the disease or disorder is Inflammatory Bowel Disease, e.g., Crohn's Disease or Ulcerative Colitis. In some embodiments, the disease or disorder is inflammation associated with an infection or injury. In other embodiments, the disease or disorder is asthma, allergy, lupus, multiple sclerosis, diabetes, e.g., type II diabetes, arthritis, e.g., rheumatoid or psoriatic. In certain embodiments, the oligonucleotide agent silences an integrin or co ligand thereof, e.g., VLA4, VCAM, ICAM. In other embodiments the oligonucleotide agent silences a selectin or co ligand thereof, e.g., P selectin, E selectin (ELAM), I selectin, P selectin glycoprotein 1 (PSGL 1). In certain embodiments, the oligonucleotide agent silences a component of the complement system, e.g., C3, C5, C3aR, C5aR, C3, convertase, and C5 convertase. In some embodiments, the oligonucleotide agent silences a chemokine or receptor thereof, e.g., TNFI, TNFJ, IL 1I, IL 1J, IL 2, IL 2R, IL 4, IL 4R, IL 5, IL 6, IL 8, TNFRI, TNFRII, IgE, SCYA11, and CCR3. In other embodiments the oligonucleotide agent silences GCSF, Gro1, Gro2, Gro3, PF4, MIG, Pro Platelet Basic Protein (PPBP), MIP 1I, MIP 1J, RANTES, MCP 1, MCP 2, MCP 3, CMBKR1, CMBKR2, CMBKR3, CMBKR5, AIF 1, or I 309.

In some aspects, the composition comprises a polymer or a polymeric micelle and an oligonucleotide agent useful for treating a subject, e.g., a human, at risk for or afflicted with a neurological disease or disorder. The method comprises providing a polymer or a polymeric micelle comprising an oligonucleotide agent, wherein said oligonucleotide is homologous to and/or can silence, e.g., by cleavage, a gene that mediates a neurological disease or disorder; and administering a therapeutically effective dose of said oligonucleotide agent to a subject, e.g., a human. In some embodiments, the disease or disorder is Alzheimer Disease or Parkinson Disease. In certain embodiments, the oligonucleotide agent silences an amyloid family gene, e.g., APP; a presenilin gene, e.g., PSEN1 and PSEN2, or I synuclein. In other embodiments, the disease or disorder is a neurodegenerative trinucleotide repeat disorder, e.g., Huntington disease, dentatorubral pallidoluysian atrophy, or a spinocerebellar ataxia, e.g., SCA1, SCA2, SCA3 (Machado Joseph disease), SCA7 or SCA8. In some embodiments, the oligonucleotide agent silences HD, DRPLA, SCA1, SCA2, MJD1, CACNL1A4, SCA7, or SCA8.

In certain aspects the composition comprises a polymer or a polymeric micelle and an oligonucleotide agent capable of cleaving or silencing more than one gene. In these embodiments, the oligonucleotide agent is selected so that it has sufficient homology to a sequence found in more than one gene, e.g., a sequence conserved between these genes. Thus in some embodiments, an oligonucleotide agent targeted to such sequences effectively silences the entire collection of genes.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. The following examples provide various illustrative embodiments of the invention as well as synthesis methods and various biological and other activity parameters. The examples, however, provide details concerning only some of the embodiments of the invention and are not intended to be limiting. It is intended that the following claims define the scope of the invention and that methods and structures within the scope of these claims and their equivalents be covered thereby.

### EXAMPLES

Throughout the description of the present invention, various known acronyms and abbreviations are used to describe monomers or monomeric residues derived from polymerization of such monomers. Without limitation, unless otherwise noted: “BMA” (or the letter “B” as equivalent shorthand notation) represents butyl methacrylate or monomeric residue derived therefrom; “DMAEMA” (or the letter “D” as equivalent shorthand notation) represents N,N-dimethylaminoethyl methacrylate or monomeric residue derived therefrom; “Gal” refers to galactose or a galactose residue, optionally including hydroxyl-protecting moieties (e.g., acetyl) or to a pegylated derivative thereof; “Nag” refers to N-acetyl galactosamine or a N-Acetyl galactosamine residue, optionally including hydroxyl-protecting moieties (e.g., acetyl) or to a pegylated derivative thereof; “HPMA” represents 2-hydroxypropyl methacrylamide or monomeric residue derived therefrom; “HPMA-E” represents 2-hydroxypropyl methacrylate or monomeric residue derived therefrom; “MAA” represents methylacrylic acid or monomeric residue derived therefrom; “MAA(NHS)” represents N-hydroxyl-succinimide ester of methacrylic acid or monomeric residue derived therefrom; “PAA” (or the letter “P” as equivalent shorthand notation) represents 2-propylacrylic acid or monomeric residue derived therefrom, “PEGMA” refers to the pegylated methacrylic monomer, CH₃O(CH₂CH₂O)ₙC(O)C(CH₃)CH₂ or monomeric residue derived therefrom (PEGMAₙ; n=7-8; n=4-5). In each case, any such designation indicates the monomer (including all salts, or ionic analogs thereof), or a monomeric residue derived from polymerization of the monomer (including all salts or ionic analogs thereof), and the specific indicated form is evident by context to a person of skill in the art.

Structures of the monomers used in the preparation of the polymers:

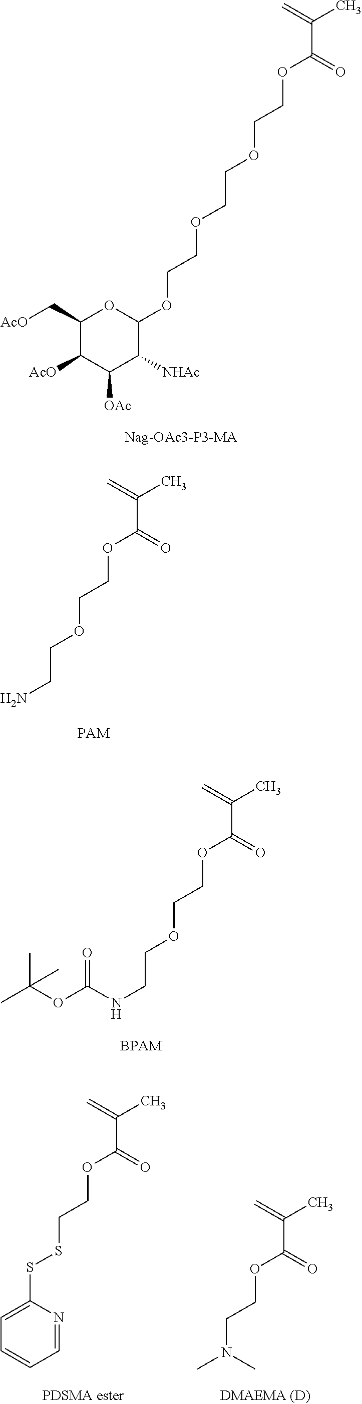

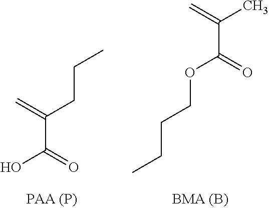

¹H NMR spectra were recorded on Bruker AV301 in deuterated solvents as indicated in each experiment at 25° C. Gel permeation chromatography (GPC) was used to determine molecular weights and polydispersities (PDI, M_{w}/Mₙ) of the macroCTAs and copolymer samples in DMF using a Viscotek GPCmax VE2001 and refractometer VE3580 (Viscotek, Houston, Tex.). HPLC-grade DMF containing 1.0 wt % LiBr was used as the mobile phase. UV/Vis spectroscopy was performed using a NanoDrop UV/Vis spectrometer (path length 0.1 cm). Particle sizes of the polymer and polymer-siRNA conjugate particles were measured by dynamic light scattering using a Malvern Zetasizer Nano ZS. Cyano-4-(ethylsulfanylthiocarbonyl) sulfanylpentanoic acid (ECT) was used as the chain transfer agent (CTA) in the synthesis of MacroCTAs, and azobisisobutyronitrile (AIBN) (Wako chemicals) was used as the radical initiator in all polymerization reactions, unless stated otherwise.

### Example 1

### Synthesis of p[Nag-P3-MA]_{15.2 KDa}-p[PAM]_{10.7 KDa}-p[D₂₅-B₄₀-P₂₅-PDSMA₁₀]_{51.0 KDa}

### Example 1-1

### Synthesis of p[Nag-OAc3-P3-MA]-MacroCTA (polymer P1)

Nag-OAc3-P3-MA monomer (2.0 g, 3.65 mmoles), ECT (21 mg, 0.07311 mmoles) need to define ECT, and AIBN (1.2 mg, 0.007311 mmoles; CTA: AIBN 10:1) and DMF (1.85 ml) were introduced under nitrogen into a sealed vial. The monomer concentration was 2 M without counting the volume of the monomer. The mixture was then degassed by bubbling nitrogen into the mixture for 30 minutes and then was placed into a reaction block (67-68° C.; stirring speed 350 rpm). After 6 hrs, the reaction was stopped by placing the vial in ice and exposing the mixtures to air. The purification of the final polymer was done by dialysis against methanol for 24 hours. The structure and composition were verified by ¹H NMR, which also verified the absence of residual vinyl groups from un-incorporated monomers. Purity of the polymer was confirmed by GPC analysis. M_{n,GPC}=22000 g/mol, dn/dc=0.058, PDI=1.15.

### Example 1-2

### Synthesis of p[Nag-OAc3-P3-MA]-b-p[BPAM] MacroCTA (polymer P2)

TABLE 1

| Name | FW (g/mol) | Equiv. | mol | Weight | Actual weight |
|---|---|---|---|---|---|
| BPAM | 273.33 | 100 | 3.3 × 10⁻³ | 0.911 g | 0.970 g |
| MacroCTA | 12000 | 1 | 3.3 × 10⁻⁵ | 0.4 g | 0.398 g |
| AIBN | 164.21 | 0.05 | 1.6 × 10⁻⁶ | 0.274 mg | 0.274 mg |

DMF = 0.9 g;
[BPAM] = 1.83M;
N₂ Purging: 30 min;
Temp. = 68° C.;
Time = 65 m
p[Nag-OAc3-P3-MA] as macroCTA;
M_{n, GPC} = 22000 g/mol

BPAM, p[Nag-OAc3-P3-MA] as macroCTA, AIBN and DMF were added to a 20 mL glass vial. The reaction vial was then degassed by bubbling nitrogen into the mixture for 30 minutes. The polymerization reaction was started by putting the reaction vial in a pre-heated reaction block at 68° C. (stirring speed 350 rpm). The reaction was allowed to proceed for 65 min (assuming 50% of conversion). The reaction was stopped by placing the vial in ice and exposing the mixture to air. The polymer was precipitated from DMF/acetone (1:1) into hexane/ether 70/30 (v/v) solvent mixture. Then, the polymer was dissolved in acetone and precipitated into hexane/ether 70/30 (two times). The resulting polymer was dried under vacuum for ˜10 hours. The structure and composition were verified by ¹H NMR. No signals of vinyl groups due to the presence of monomer impurities were observed. The di-block copolymer was soluble in acetone, DMF, CDCl₃, etc. The dn/dc was determined from the area under the RI traces at different injection volume (40, 60, 80, and 100 uL). The line passed through the origin of a linear plot of refractive index (RI) vs. concentration; dn/dc=0.05175; Mₙ=37000 g/mol; PDI=1.147. The ¹H NMR spectrum confirmed the presence of BPAM, and was consistent for the structure. The GPC RI trace was monomodal.

### Example 1-3

### Synthesis of polymer P2: p[Nag-OAc3-P3-MA]-b-p[BPAM]-b-p[BMA-PAA-DMAEMA-PDSMA] Polymer P3

TABLE 2

| Name | FW (g/mol) | Equiv. | mol | Weight | Actual weight |
|---|---|---|---|---|---|
| BMA | 142.20 | 196.8 | 2.127 × 10⁻³ | 0.302 g | 0.306 g |
| PAA | 114.14 | 123 | 1.329 × 10⁻³ | 0.152 g | 0.153 g |
| DMAEMA | 157.21 | 123 | 1.329 × 10⁻³ | 0.209 g | 0.210 g |
| PDSMA | 255.36 | 49.2 | 5.318 × 10⁻⁴ | 0.136 g | 0.155 g |
| MacroCTA | 37000 | 1 | 1.081 × 10⁻⁵ | 0.4 g | 0.400 g |
| AIBN | 164.21 | 0.1 | 1.081 × 10⁻⁶ | 0.18 mg | 0.18 mg |

DMF = 0.88 g;
N₂ purging: 30 min;
Temp. = 68° C.;
polymerization time = 10 h
[Monomer]:[CTA] = 492:1;
[CTA]:[AIBN] = 10:1
[BMA]:[PAA]:[DMAEMA]:[PDSMA] = 40:25:25:10

BMA and DMAEMA were purified by passing through a basic alumina column just before use. BMA, PAA, PDSMA, DMAEMA, [Nag-OAc3-P3-MA]-b-p[BPAM]-macroCTA (M_{n,GPC}=37000 g/mol; PDI=1.147), AIBN and DMF were added to a 20 mL glass vial in the amounts indicated in Table 1. The total monomer concentration was 3M. The reaction mixture was then degassed by bubbling nitrogen into the mixture for 30 minutes. The polymerization reaction was started by putting the reaction vial to a pre-heated reaction block at 68° C. (stirring speed 350 rpm). The reaction was allowed to proceed for 10 hours (assuming 50% of conversion). The reaction was stopped by placing the vial in ice and exposing the mixture to air. The polymer was precipitated from DMF/acetone (1:1) into hexane/ether 70/30 (v/v) solvent mixture. Then, the polymer was dissolved in acetone and precipitated into hexane/ether 70/30 (two times). The resulting polymer was dried under vacuum for ˜10 hours. The structure and composition were verified by ¹H NMR. No vinyl groups due to impurities of unreacted monomers were observed. The final triblock copolymer was soluble in acetone, DMF, Ethanol, CDCl₃, etc. The dn/dc was determined from the area under the RI traces at different injection volume (60, 80, 100, and 120 uL). The line passed through the origin of a linear plot of RI vs. concentration; dn/dc=0.060683; Mₙ=88,000 g/mol; PDI=2.00. ¹H NMR spectrum confirmed the presence of all the monomeric units in the polymer. The GPC RI trace was bimodal.

Boc protecting groups were removed by treating the polymer with TFA in dichloromethane (70/30, 1 hr), followed by precipitation with Et₂O. The resulting solid was taken up in DMF and preciptated with Et₂O (2 times) and dried under vacuum.

### Example 1-4

### Preparation of the polymer P4

p[Nag-P3-MA]_{15.2 KDa}-p[PAM]_{10.7 KDa}-p[D₂₅-B₄₀-P₂₅-PDSMA₁₀]_{51.0 KDa}

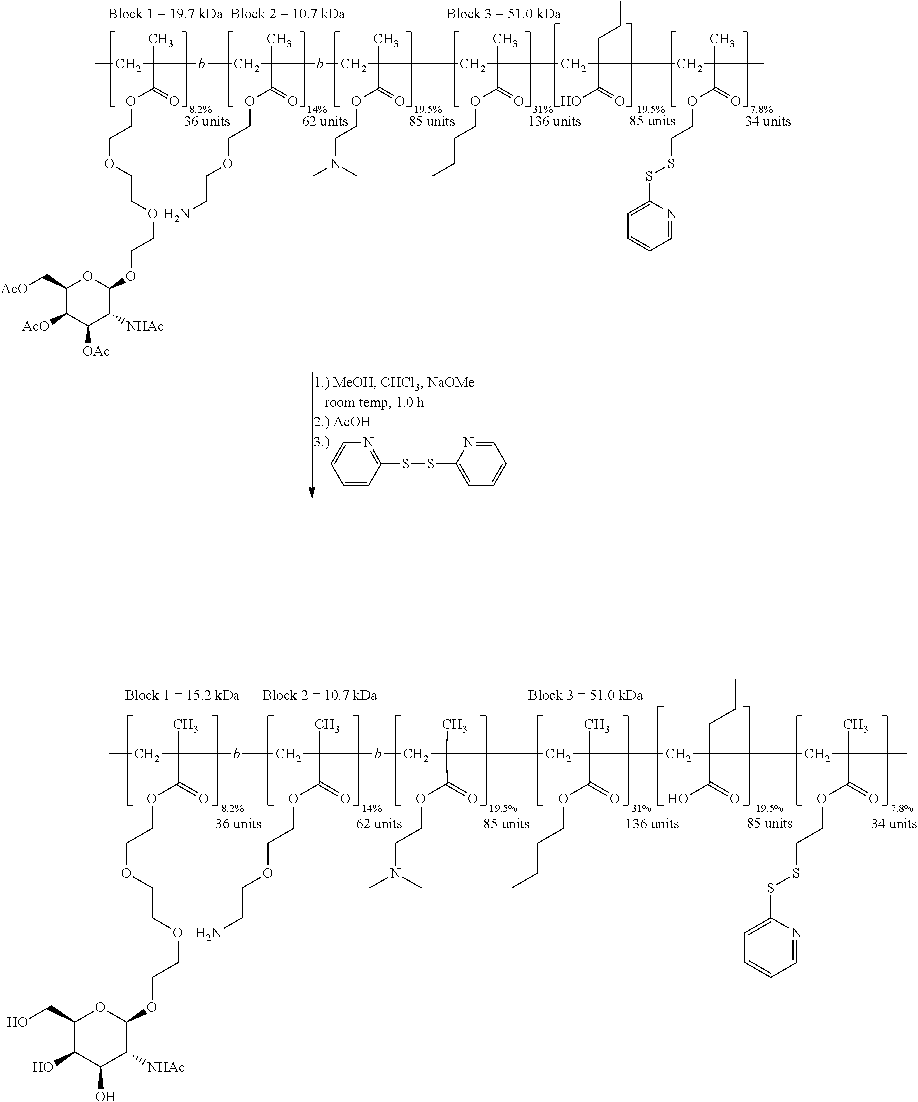

To a 50 mL one-neck round-bottom flask was added the polymer P3 (100 mg, 538 μmol polymer containing 44 μmol galactose and 42 μmol pyridyl disulfide groups) followed by anhydrous methanol (2.9 mL), anhydrous chloroform (1.5 mL) and sodium methoxide (143 μL, 14.3 mg, solution of 100 mg/mL in anhydrous MeOH, 264 μmol, 6 equivalents relative to galactose). This mixture was stirred under an atmosphere of argon at room temperature for 1.0 hour. Then glacial acetic acid (7.56 μL, 132 μmol, 3 equivalents relative to galactose) was added to the reaction followed by 2,2′-dipyridyl disulfide (18.5 mg, 84 μmol, 2 equivalents relative to pyridyl disulfide). This mixture was stirred at room temperature for 1.0 hour under a flow of argon gas. After the capping with 2,2′-dipyridyl disulfide for 1.0 hour the reaction was then precipitated into Et₂O (2×50 mL). After the polymer was precipitated it was then transferred to a round-bottom flask and the solvent was evaporated using a rotary evaporator providing 65.1 mg (69%) of a clear film. NMR analysis of the purified polymer was performed using CD₃OD indicating that the precipitation process did not remove all NaOAc from the reaction mixture judging by the presence of a singlet at 1.9 ppm. After precipitation the polymer it was dissolved in absolute EtOH at 100 mg/mL and then directly used in conjugation reactions with siRNA. The NaOAc impurity was present in the final product and in siRNA formulations using this polymer.

### Example 2

### Synthesis of p[DMAEMA]_{14 KDa}-p[PAA₂₅-BMA₅₀-PDSMA₂₅]_{40 KDa}

### Example 2-1

### Synthesis of p[DMAEMA] MacroCTA

DMAEMA (Aldrich, 98%) was passed through a small alumina column just before use to remove the inhibitor. DMAEMA (3.01 g, 150 eq.), ECT (33.8 mg, 1 eq.) and AIBN (2.3 mg, 0.1 eq.) were placed in a glass vial equipped with a rubber septa. The reaction vial was purged with dry nitrogen for 30 min, then placed in a preheated reaction block at 60° C. (steering speed 350 RPM). The reaction was allowed to proceed for 2 hrs 20 min. The reaction was stopped by placing the vial in ice and exposing the mixture to air. The purification of the polymer was done by precipitation of the product from acetone into hexane/ether 80/20 (six times). The polymer was dried under vacuum for 8 h at RT. M_{n,GPC}=14,000 g/mol; PDI=1.123.

### Example 2-2

### Synthesis of p[DMAEMA]_{14 KDa}-p[PAA₂₅-BMA₅₀-PDSMA₂₅]_{40 KDa} Polymer P5

TABLE 3

| | | | | | Actual |
|---|---|---|---|---|---|
| Name | FW (g/mol) | Equiv. | mol | Weight | weight |
| BMA | 142.20 | 171 | 4.071 × 10⁻³ | 0.579 g | 0.583 g |
| PAA | 114.14 | 85.5 | 2.035 × 10⁻³ | 0.232 g | 0.240 g |
| PDSMA | 255.36 | 85.5 | 2.035 × 10⁻³ | 0.520 g | 0.537 g |
| pDMAEMA | 14000 | 1 | 2.381 × 10⁻⁵ | 0.333 g | 0.333 g |
| AIBN | 164.21 | 0.1 | 2.381 × 10⁻⁶ | 0.390 mg | 0.390 mg |

DMF = 1.24 g;
N₂ purging: 30 min;
Temp. = 68° C.;
polymerization time = 10 h
[BMA]:[PAA]:[PDSMA] = 50:25:25

pDMAEMA-macroCTA (PD-01-34B) was used for this experiment. M_{n,GPC}=14,000 g/mol; PDI=1.123. BMA was purified by passing through a basic alumina column just before use. BMA, PAA, PDSMA-E, pDMAEMA-macroCTA (CTA: Monomers=1:342), AIBN (CTA: AIBN=10:1) and DMF were taken into a 20 mL glass vial in the amounts indicated in Table 3. The total monomer concentration was 3 M. The reaction mixture was then degassed by bubbling nitrogen into the mixture for 30 minutes. The polymerization reaction was started by putting the reaction vial in a pre-heated reaction block at 68° C. (stirring speed 350 rpm). The reaction was allowed to proceed for 10 hours (assuming 50% of conversion). The reaction was stopped by placing the vial in ice and exposing the mixture to air. The purification of the polymer was done by precipitation of the product from DMF/acetone (1:1) into hexane/ether 75/25 (four times). The resulting polymer was dried under vacuum for ˜10 hours. The structure and composition were verified by ¹H NMR. The dn/dc was determined from the area under the RI traces at different injection volume (60, 80, 100, and 120 uL). The line passed through the origin of a linear plot of RI vs. concentration: dn/dc=0.072188; Mₙ=54000 g/mol; PDI=1.76. From ¹H NMR spectrum, composition was determined. In the copolymer: % BMA=57.0%, % PAA=20.3% and % PDSMA-E=22.7%. The GPC RI trace was bimodal.

### Example 3

### Synthesis of b[DMAEMA]_{14.6 KDa}-b[DMAEMA₂₅-BMA₄₀-PAA₂₅-PDSMA₁₀]_{41.4 KDa}

### Example 3-1

### Synthesis of polymer

### b[DMAEMA]_{14.6 KDa}-b[DMAEMA₂₅-BMA₄₀-PAA₂₅-PDSMA₁₀]_{41.4 KDa}

p[DMAEMA]-MacroCTA was obtained as described in Example 2-1, M_{n,GPC}=14,570 g/mol; PDI=1.128.

TABLE 4

| | | | | | Actual |
|---|---|---|---|---|---|
| Name | FW (g/mol) | Equiv. | mol | Weight | weight |
| BMA | 142.20 | 155.2 | 4.644 × 10⁻³ | 0.660 g | 0.665 g |
| PAA | 114.14 | 97.0 | 2.903 × 10⁻³ | 0.331 g | 0.334 g |
| DMAEMA | 157.21 | 97.0 | 2.903 × 10⁻³ | 0.456 g | 0.455 mg |
| PDSMA | 255.36 | 38.8 | 1.161 × 10⁻³ | 0.296 g | 0.295 mg |
| pDMAEMA | 14570 | 1 | 2.992 × 10⁻⁵ | 0.436 g | 0.436 mg |
| AIBN | 164.21 | 0.1 | 2.992 × 10⁻⁶ | 0.490 mg | 0.500 mg |

DMF = 1.9 g;
N₂ purging: 30 min;
Temp. = 68° C.;
polymerization time = 10 h
[BMA]:[PAA]:[DMAEMA]:[PDSMA] = 40:25:25:10
[Monomer]:[CTA] = 388:1;
[CTA]:[AIBN] = 10:1

BMA and DMAEMA were purified by passing through a basic alumina column just before use. BMA, PAA, PDSMA, DMAEMA, pDMAEMA-macroCTA, AIBN and DMF were taken in a 20 mL glass vial in the amounts indicated in table 4. The total monomer concentration was 3 M. The reaction mixture was degassed by bubbling nitrogen into the mixture for 30 minutes. The polymerization reaction was started by putting the reaction vial in a pre-heated reaction block at 68° C. (stirring speed 350 rpm). The reaction was allowed to proceed for 10 hours (assuming 50% of conversion) and then stopped by placing the vial in ice and exposing the mixture to air. The polymer was precipitated from DMF/acetone (1:1) into hexane/ether 75/25 (v/v) solvent mixture. Then, the polymer was dissolved in acetone and precipitated into hexane/ether 75/25 (two times). The resulting polymer was dried under vacuum for ˜10 hours. The structure and composition were verified by ¹H NMR. No vinyl groups of unreacted monomers were observed in the ¹H NMR spectrum. The final diblock copolymer was soluble in acetone, DMF, Ethanol, CDCl₃. dn/dc=0.065537, Mₙ=56000 g/mol; PDI=2.45. From ¹H NMR spectrum, composition was determined for the second block: % BMA=47.0%, % PAA=23.8%, % DMAEMA=21.7%, and % PDSMA=7.5%.

Polymer particle size was studied by DLS measurements. The polymer was dissolved in ethanol at 50 mg/mL concentration, and 20 uL of this solution was added to the 980 uL PB pH 7.4 to obtain 1 mg/mL solution. Particle size was measured at different dilutions. Following Z-average diameters (PDI) were obtained: 49.5 nm (0.092) at 1 mg/mL, 50.9 nm (0.150) at 0.2 mg/mL, 56.2 nm (0.204) at 0.04 mg/mL, 62.5 nm (0.145) at 0.008 mg/mL, 122.9 nm (0.494) at 0.0016 mg/mL, 282.7 nm (0.808) at 0.00032 mg/mL.

### Example 4

### Synthesis of b[Nag-P2-MA]_{33 KDa}-b[DMAEMA]_{8.1 KDa}-b[DMAEMA₂₅-BMA₄₀-PAA₂₅-PDSMA₁₀]_{35 KDa}

### Example 4-1

### Synthesis of b[Nag-OAc3-P2-MA]_{33kDa} MacroCTA

Nag-OAc3-P2-MA monomer (2.5 g, 4.967 mmoles), ECT (16.4 mg, 0.0621 mmoles), AIBN (0.3 mg, 0.00155 mmoles; CTA:AIBN 40:1) and DMF (1.65 ml) were introduced in a sealed vial under nitrogen. The total monomer concentration was 3 M without counting the volume of the Nag-OAc3-P2-MA monomer. The mixture was degassed by bubbling nitrogen into it for 30 minutes and placed in a pre-heated reaction block (67-68° C.; stirring speed 350 rpm). The reaction was allowed to proceed for 3 hrs 30 min and was stopped by placing the vial in ice and exposing the mixtures to air. The purification of the final polymer was done by dialysis against methanol for 24 hours. No vinyl groups due to the presence of un-reacted monomer were observed in the NMR spectrum. Mn=33400 g/mol; dn/dc=0.050182; PDI=1.17 (by GPC).

### Example 4-2

### Synthesis of b[Nag-OAc3-P2-MA]_{33kDa}-b-[DMAEMA]_{8.1 KDa} MacroCTA

DMAEMA (0.39 g, 2.47 mmoles), MacroCTA (0.55 g, 0.0165 mmoles; CTA:monomers 1:150), and AIBN (0.271 mg, 0.00165 mmoles; CTA: AIBN 10:1) and DMF (0.4 ml) were introduced in a sealed vial under nitrogen. The total monomer concentration was 3 M. The mixture was degassed by bubbling nitrogen into it for 30 minutes and placed in a pre-heated reaction block (67-68° C.; stirring speed 350 rpm). The reaction was allowed to proceed for 2 hrs (assuming 40% of conversion) and was stopped by placing the vial in ice and exposing the reaction mixture to air. The purification of the polymer was done by dialysis against methanol for 24 hours. The methanol was removed after and the polymer dried. Mn=41000 g/mol; dn/dc=0.054636; PDI=1.15 (by GPC).

### Example 4-3

### Synthesis of b[Nag-P2-MA]_{33 KDa}-b-[DMAEMA]_{8.1 KDa}-b-[DMAEMA₂₅-BMA₄₀-PAA₂₅-PDSMA₁₀] Polymer P7

BMA (0.207 g, 1.456 mmoles), PAA (0.104 g, 0.91 mmoles), DMAEMA (0.143 g, 0.91 mmoles), PDSMA (0.093 g, 0.364 mmoles), b[Nag-Ac—P2-MA]₃₃-b-[DMAEMA]₈ macroCTA (0.32 g, 0.0078 mmoles; 1:467 CTA:Monomers), AIBN (0.128 mg, 0.00078 mmoles; CTA:AIBN 10:1) and DMF (0.63 ml) were introduced under nitrogen into a sealed vial. The total monomer concentration was 3 M. The mixture was degassed by bubbling nitrogen into it for 30 minutes, and then placed after in a pre-heated reaction block (67-68° C.; stirring speed 350 rpm). The reaction was left 10 hrs (assuming 50% of conversion). The reaction was stopped by placing the vial in ice and exposing the mixture to air. The purification of the polymer was done by precipitation from acetone/DMF 1:1 into hexane/ether 75/25 (four times). The resulting polymer was dried under vacuum for at least 8 hours.

The resulting polymer (0.35 g, 0.143 mmoles of Nag pendant groups) was dissolved in a 3 ml mixture of CHCl₃: Methanol 1:2. The solution was stirred at room temperature until dissolution. A solution of sodium methoxide in methanol (1M, 1.1424 ml, 1.1424 mmoles; 1:2 Ac:NaOMe) was added to the mixture. The mixture was left stirring under nitrogen for 1 hour 15 minutes. The reaction was stopped and the mixture was dialysed against water for 1 day using 1K membrane. The polymer was recovered by lyophilization. GPC following third block synthesis and deprotection resulted in a slightly bi-modal polymer distribution, thereby making molecular weight determination not possible.

### Example 5

### Synthesis of p[Nag-P3-MA]_{13.1 KDa}-b-p[D₁₇-B₃₅-P₁₇-PDSMA_{6.7}]_{25.0 KDa}

### Example 5-1

### Synthesis of p[Nag-OAc3-P3-MA] MacroCTA

Nag-OAc3-P3-MA monomer (3.2 g, 5.848 mmoles), ECT (30.8 mg, 0.117 mmoles), and AIBN (0.96 mg, 0.005848 mmoles; CTA:AIBN 20:1) and DMF (2.92 ml) were introduced into a sealed vial under nitrogen. The monomer concentration was 2 M without counting the volume of the monomer. The mixture was then degassed by bubbling nitrogen into it for 30 minutes and placed in a preheated reaction block (67-68° C.; stirring speed 350 rpm). The reaction was allowed to proceed for 6 hrs. The reaction was stopped by placing the vial in ice and exposing the mixtures to air. The purification of the final polymer was done by dialysis against methanol for at least 24 hours. The product was characterized by ¹H NMR and GPC. dn/dc=0.057698, Mₙ=17100 g/mol; PDI=1.16.

### Example 5-2

### Synthesis of p[Nag-OAc3-P3-MA]_{17.1 KDa}-b-[D₂₅-B₄₀P₂₅-PDSMA₁₀]_{22.8 KDa} (Polymer P8)

BMA (0.276 g, 1.94 mmoles), PAA (0.139 g, 1.215 mmoles), DMAEMA (0.191 g, 1.215 mmoles), PDSMA (0.124 g, 0.486 mmoles), p[Nag-OAc3-P3-MA] MacroCTA (0.250 g, 0.0146 mmoles; 1:467 CTA:Monomers), AIBN (0.24 mg, 0.00146 mmoles; CTA:AIBN 10:1) and DMF (0.83 ml) were introduced under nitrogen in a sealed vial. The total monomer concentration was 3 M. The mixture was degassed by bubbling nitrogen into it for 30 minutes and then placed in a pre-heated block (67-68° C.; stirring speed 350 rpm). The reaction was allowed to proceed for 10 hrs (assuming 50% of conversion). The reaction was stopped by placing the vial in ice and exposing the mixture to air. The purification of the polymer was done by precipitation from Acetone/DMF 1:1 into hexane/ether 75/25 (four times). The resulting polymer was dried under vacuum for at least 8 hours. The product was characterized by ¹H NMR and GPC; Mn=29260 g/mol (not corrected); PDI=2.06; dn/dc=0.06339. B/P/D/PDSMA 40/25/25/10 (theoretical, based on monomer feed ratio).

### Example 5-3

### Deprotection of Polymer P8 to p[Nag-P3-MA]_{13.1 KDa}-b-[D₁₇-B₃₅-P₁₇-PDSMA_{6.7}]_{22.8 KDa}

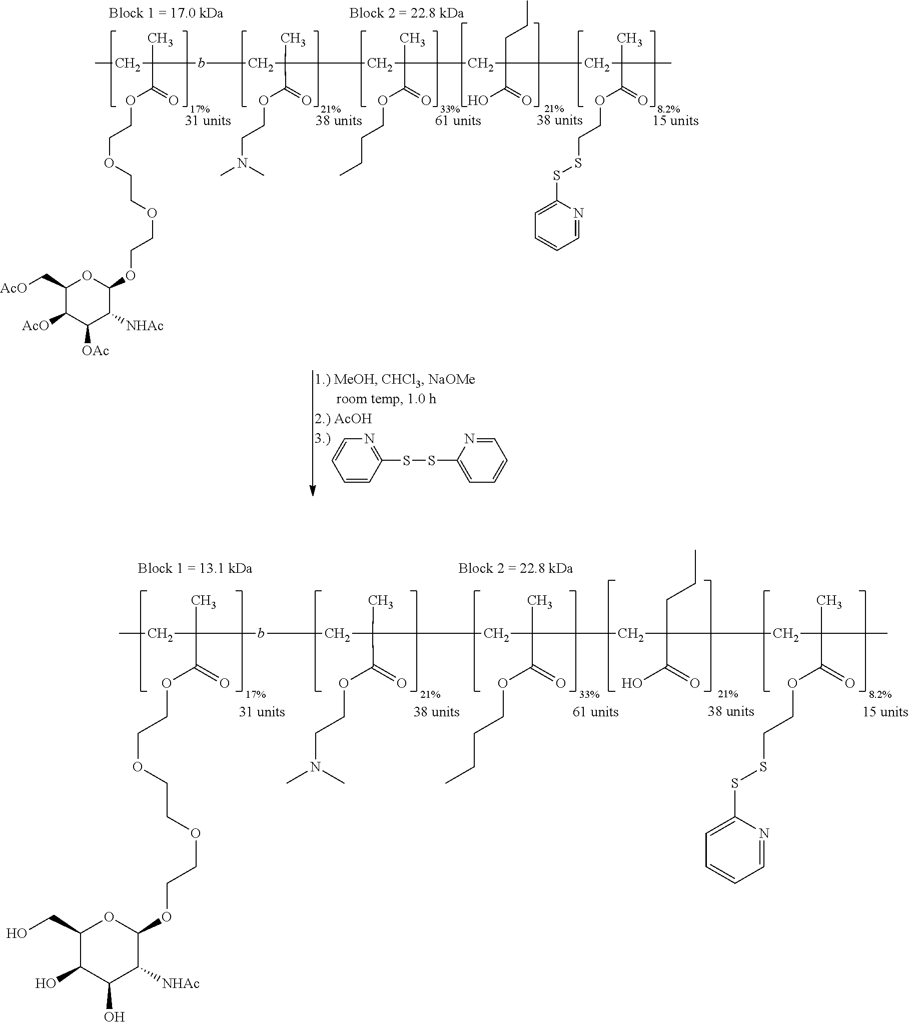

To a 50 mL one-neck round-bottom flask was added the polymer P8 (360 mg, 1653 μmol polymer, 281 μmol galactose, 136 μmol pyridyl disulfide) followed by anhydrous methanol (2.09 mL), anhydrous chloroform (1.5 mL) and sodium methoxide (910 μL, 91.0 mg, solution of 100 mg/mL in anhydrous MeOH, 1686 μmol, 6 equivalents relative to N-acetyl galactosamine). This mixture was stirred under an atmosphere of argon at room temperature to 1.0 hour. Then glacial acetic acid (48.3 μL, 843 μmol, 3 equivalents relative to galactose) was added to the reaction followed by 2,2′-dipyridyl disulfide (59.8 mg, 272 μmol, 2 equivalents relative to pyridyl disulfide on polymer). This mixture was stirred at room temperature for 2.0 hour under a flow of argon gas. After capping with 2,2′-dipyridyl disulfide for 2.0 hour the reaction was diluted with MeOH (5 mL) and filtered via gravity filtration. The filtered reaction solution was transferred to a dialysis membrane with a 2000 g/mol molecular weight cut off (Spectrum Labs, Spectra/Por Dialysis Membrane MWCO: 2000) and dialyzed against MeOH (2×500 mL) over 24 hours. After the dialysis was complete the polymer was transferred to a round-bottom flask and the solvent was evaporated using a rotary evaporator providing 271.5 mg (87%) of the product as a clear film. Polymer ¹H NMR analysis performed in CD₃OD confirmed the structure. After dialysis the polymer was dissolved in absolute EtOH at 100 mg/mL concentration and then directly used in conjugation reactions with siRNA. B/P/D/PDSMA ratio was determined from the NMR data.

### Example 6

### Synthesis of p[Nag-P3-MA]_{15.2 KDa}-p[PAM]_{10.7 KDa}-p[D25-B40-P25-PDSMA10]_{51.0 KDa} (Polymer 9) by deprotection of polymer P2

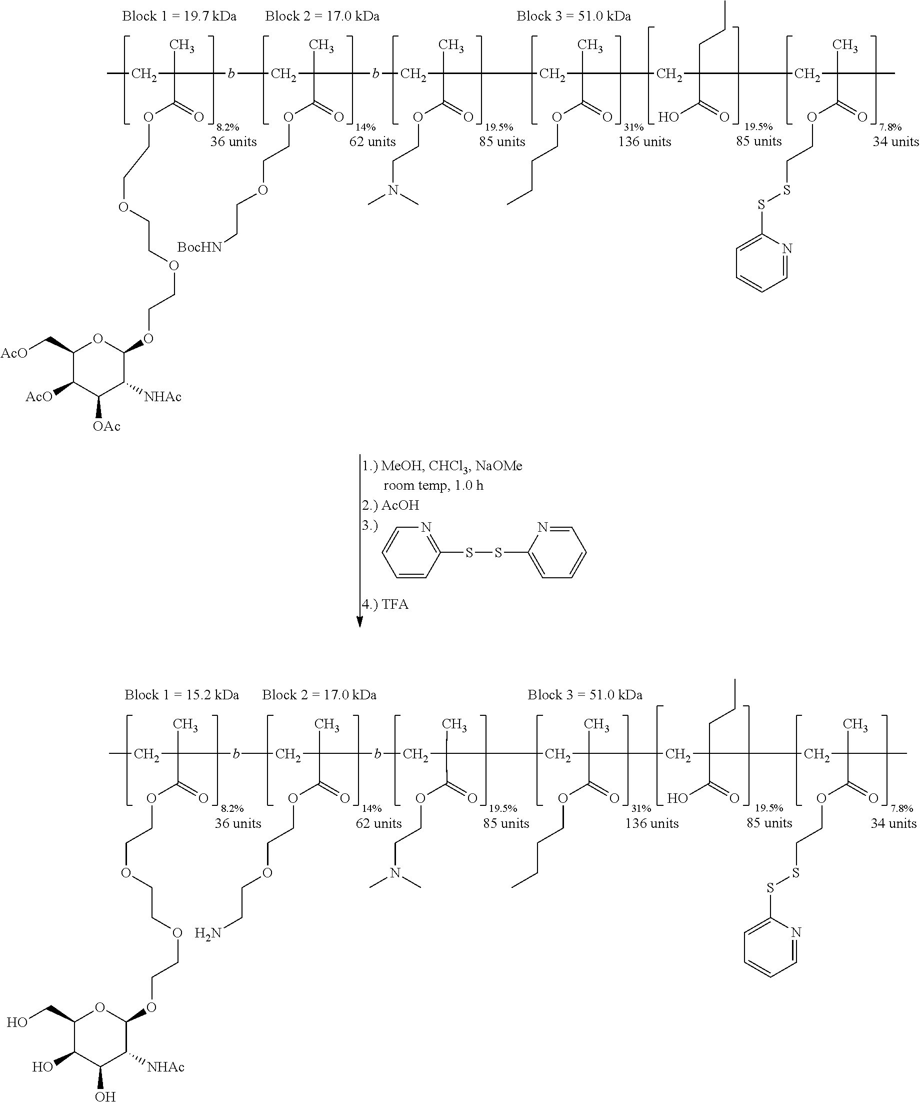

To a 50 mL one-neck round-bottom flask was added the polymer P2 (100 mg, 500 μmol polymer, 41 μmol galactose, 39 μmol pyridyl disulfide) followed by anhydrous methanol (2.9 mL), anhydrous chloroform (1.5 mL) and sodium methoxide (133 μL, 13.3 mg, solution of 100 mg/mL in anhydrous MeOH, 246 μmol, 6 equivalents relative to Nag-Ac residues). This mixture was stirred under an atmosphere of argon at room temperature for 1.0 hour. Then glacial acetic acid (7.03 μL, 123 μmol, 3 equivalents relative to Nag-Ac residues) was added to the reaction followed by 2,2′-dipyridyl disulfide (17.2 mg, 78 μmol, 2 equivalents relative to pyridyl disulfide). This mixture was stirred a room temperature for 1.0 hour under a flow of argon gas. After capping with 2,2′-dipyridyl disulfide for 1.0 hour the reaction was diluted with MeOH (5 mL) and filtered via gravity filtration. The filtered reaction solution was transferred to a dialysis membrane with a 2000 g/mol molecular weight cut off (Spectrum Labs, Spectra/Por Dialysis Membrane MWCO: 2000) and dialyzed against MeOH (2×500 mL) over 24 hours. After the dialysis was complete the polymer was transferred to a round-bottom flask and the solvent was evaporated using a rotary evaporator providing the crude product.

To the crude product (ca. 95 mg) was added trifluoroacetic acid (2.0 mL, TFA) and the mixture was stirred at room temperature for 2.0 hours under an inert atmosphere. After 2.0 hours the reaction mixture was rotary evaporated providing crude product. All the TFA was removed from the crude product by placing the compound on a high vacuum line (ca. 0.5 mm Hg) overnight. The next day the crude material was dissolved in MeOH (5.0 mL) and H₂O (1.0 mL) then treated with Dowex 1X2-400 (500 mg, washed with MeOH and CH₂Cl₂ then air dried prior to use). After stirring the mixture for 2.0 hours at room temperature the Dowex was removed via gravity filtration. The filtered reaction solution was then transferred to a dialysis membrane with a 2000 g/mol molecular weight cut off (Spectrum Labs, Spectra/Por Dialysis Membrane MWCO: 2000) and dialyzed against MeOH (1×1000 mL) for over 24 hours. After the dialysis was complete the polymer was transferred to a round-bottom flask and the solvent was evaporated using a rotary evaporator providing 69.3 mg (79%) of a clear film. ¹H NMR analysis of the purified product was performed in CD₃OD and confirmed the structure.

Although the pyridyl disulfide functionality on the polymer was easily detected by NMR spectroscopy this functionality was also observed and quantified using UV/Vis spectroscopy. The final polymer (70 mg) was dissolved in EtOH (700 μL) providing a polymer solution at a concentration of 100 mg/mL. An aliquot of this polymer solution (10 μL, 1.0 mg, 5.7 μmol) was treated with an aqueous solution of 1.0 M dithiothreitol (10 μL, 10.0 μmol, DTT) for 10 min before being diluted with H₂O (80 μL) giving a reduced polymer solution of 0.057 M. Since the expected incorporation of pyridyl disulfide should be 7.8% in the polymer one would expect the concentration of the leaving group pyridine-2-thione should be 4.44 mM (i.e., 0.057×0.078=0.00445 M) after the polymer is treated with DTT as described above. Since the molar absorptivity of pyridine-2-thione at 343 nm is ε=8.08×10³ M⁻¹ cm⁻¹ (Hermanson, G. T. Bioconjugate Techniques, 1996, 1^{St} ed. Academic Press, an imprint of Elsevier, page 66) and the path length of cuvette used was 0.1 cm the expected absorption for the reduced polymer solution above should be A=3.59 (i.e., A=8.08×10³ M⁻¹ cm⁻¹×0.1 cm×0.00445 M). After analysis of the reduced polymer solution by UV/Vis the experimentally determined absorption was A=2.044 at 343 nm. This demonstrated the presence of the pyridyl disulfide functionality on the polymer at the actual concentration of 4.44% total pyridyl disulfide incorporation in the polymer.

### Example 7

### Synthesis of p[PEGMA-PDSMA]-p[BMA-PAA-PAM]

### Example 7-1

### Synthesis of p[PEGMA-PDSMA] MacroCTA

TABLE 5

| Name | FW (g/mol) | Equiv. | mol | Weight | Actual weight |
|---|---|---|---|---|---|
| PEGMA | 475 | 40 | 3.712 × 10⁻³ | 1.763 g | 1.783 g |
| PDSMA | 255.36 | 10 | 9.279 × 10⁻⁴ | 0.237 g | 0.234 g |
| ECT | 263.4 | 1 | 9.279 × 10⁻⁵ | 24.40 mg | 24.500 mg |
| AIBN | 164.21 | 0.05 | 4.640 × 10⁻⁶ | 0.762 mg | 0.764 mg |

DMF = 2.0 g;
N₂ Purging: 30 min;
conduct polymerization at 68° C.;
polymerization time = 4 hr 15 m
[PEGMA]:[PDSMA] = 80:20

Reaction was performed under N₂ at 68° C. and stopped after 4 h 15 min. Conv.=30.0%. Mₙ=15500 g/mol; PDI=1.35; dn/dc=0.0511. From ¹H NMR spectrum, the composition was determined: % PEGMA=80.0% and % PDSMA=20.0%.

### Example 7-2

### Synthesis of p[PEGMA-PDSMA]-p[BMA-PAA-BPAM]Polymer P10

TABLE 6

| | FW | | | | Actual |
|---|---|---|---|---|---|
| Name | (g/mol) | Equiv. | mol | Weight | weight |
| BMA | 142.20 | 185 | 2.7094 × 10⁻³ | 0.3853 g | 0.3865 g |
| PAA | 114.14 | 92.5 | 1.355 × 10⁻³ | 0.155 g | 0.155 g |
| BPAM | 273.33 | 92.5 | 1.355 × 10⁻³ | 0.370 g | 0.371 g |
| p[PEGMA- | 15500 | 1 | 1.465 × 10⁻⁵ | 0.227 g | 0.227 g |
| PDSMA] | | | | | |
| AIBN | 164.21 | 0.1 | 1.465 × 10⁻⁶ | 0.241 mg | 0.242 mg |

DMF = 0.81 g;
N₂ Purging: 25 min;
Temp. = 68° C.;
polymerization time = 10 h;
[Monomer]:[CTA] = 370:1;
[CTA]:[AIBN] = 10:1;
[BMA]:[PAA]:[BPAM] = 50:25:25

BMA was purified by passing through a basic alumina column just before use. MacroCTA was placed in a 20 mL reaction glass vial. The required amount of DMF and AIBN stock solution in DMF were added to the vial. When the MacroCTA was dissolved, the required amounts of BMA, PAA and BPAM were added to the reaction vial. The monomer concentration was 3 M. The reaction mixture was then degassed by bubbling nitrogen into it for 30 minutes. The polymerization reaction was conducted for 10 h by putting the reaction vial in a pre-heated reaction block at 68° C. The reaction was stopped by placing the vial in ice and exposing the mixture to air. The polymer was precipitated from DMF into hexane/ether 75/25. Filtrate was colorless indicating no decomposition of PDS groups to 2-pyridine thione. Then, the polymer was dissolved in acetone and precipitated into hexane/ether 75/25 (two times). The resulting polymer was dried under vacuum for ˜10 h. The dn/dc was determined from the area under the RI traces at different injection volume (60, 80, 100, 120 uL). The line passes through the origin of a linear plot of RI vs. concentration; Mₙ=37000 g/mol; PDI=1.22; dn/dc=0.0560.

### Example 7-3

### Deprotection of Polymer P10 to p[PEGMA-PDSMA]-p[BMA-PAA-PAM] [PD-02-15] (Polymer P11)

The PDS group content was determined in Polymer P10 using UV/Vis. The polymer (22.5 mg) was dissolved in EtOH (0.9 mL) providing a polymer solution at a concentration of 25 mg/mL. An aliquot of this polymer solution (5 uL, 0.125 mg, 0.553

umol) was treated with an aqueous solution of 1.0 M dithiothreitol (10 uL, 10.0 umol, DTT) for 10 min before being diluted with H₂O (85 uL) giving a reduced polymer solution of 0.00553 M (0.553 umol/100 uL=0.00553 mol/L). Since the expected incorporation of pyridyl disulfide should be 4.3% in the polymer one would expect the concentration of the leaving group pyridine-2-thione should be 0.23779 mM (i.e., 0.00553×0.043=0.00023779 M) after the polymer is treated with DTT as described above. Using the molar absorptivity of pyridine-2-thione at 343 nm is e=8.08×10³ M⁻¹ cm⁻¹ (Hermanson, G. T. Bioconjugate Techniques, 1996, 1^{st} ed. Academic Press, an imprint of Elsevier, page 66) and the path length of the cuvette used 0.1 cm, the expected absorption for the reduced polymer solution above was A=0.192 (i.e., A=8.08×10³ M⁻¹ cm⁻¹×0.1 cm×0.00023779 M). After analysis of the reduced polymer solution by UV/Vis the experimentally determined absorption was A=0.185 at 343 nm. This demonstrated the presence of pyridyl disulfide functionality in the polymer with the actual concentration of 4.15% total pyridyl disulfide incorporation in the polymer (vs 4.3% theoretically calculated).

Trifluoroacetic acid (0.232 mL, 20 equiv. per Boc equiv.; MW of TFA=114.02 g/mol) was added to a solution of p[PEGMA-PDSMA]-p[BMA-PAA-BPAM] (0.1805 g) in dichloromethane (10 mL) cooled in an ice/water bath while purging with dry nitrogen. The reaction mixture was warmed to room temperature and stirred for 5 h.

After 5 h, 2 mL of methanol was added, and the mixture was stirred for 10 min. Approximately 6 mL dichloromethane was evaporated from the reaction mixture. The polymer was precipitated into hexane/ether 75/25 (v/v) solvent mixture. The resulting polymer was dried under vacuum for ˜10 hours and analyzed by ¹H NMR spectroscopy. The resonance signal of the Boc group in P10 at 1.46 ppm disappeared after deprotection reaction.

### Example 8

### Conjugation of siRNA to Polymer P11 and Knockdown Activity of the siRNA Polymeric Conjugates

Preparation of thiolated siRNA was as follows. To a 15 mL Falcon tube was added tris(2-carboxyethyl)phosphine hydrochloride (1.0 mg, 3.5 μmol, TCEP) followed by NaHCO₃ (1.2 mg, 14.0 μmol), H₂O (500 μL) and ApoB-SSC₆OH duplex (5.0 mg, Agilent Technologies). This mixture was allowed to stand at room temperature. After 30 min, 5.0 M NaCl (20.0 μL) was added followed by cold (−20° C.) 100% EtOH (5.0 mL). The mixture was placed into a −80° C. freezer for 30 min to achieve complete RNA precipitation. The Falcon tube was then centrifuged to pellet the RNA. The mother liquor was removed and the remaining RNA pellet was triturated using cold (+4° C.) 70% EtOH (1×1.0 mL). The remaining RNA pellet was then dissolved in isotonic glucose (5.0 mL, 5.05 wt % glucose, 10 mM HEPES, pH 7.4) to give an aqueous RNA solution with RNA concentration at 0.7 μg/μL (by UV analysis).

Polymer stocks were weighed fresh and brought up to 100 mg/ml in 100% EtOH with vigorous shaking. Polymer solutions are very viscous at this concentration. Reduced siRNA was adjusted to 0.7 μg/μL mg/ml in Isotonic Glucose solution (ITG), pH 7.4 and diluted, as indicated in protocol, with ITG, pH9. To this, the EtOH stock of polymer was added. The solution was vortexed and allowed to conjugate overnight. The solutions were adjusted using ITG, pH4 (brought down from pH6.5 using acetic acid). The knockdown activity of the resulting conjugated siRNA formulations was tested as described below.

Particle size measurements were taken on the final conjugated siRNA formulation (approximately 10 mg/ml polymer): Z average 26.5 nm, PDI 0.334.

The conjugation reactions were analyzed by gel electrophoresis (20% polyacrylamide, 1×TBE gel from Invitrogen, 1×TBE buffer for ca. 1 h at 200 V, stained in 50 mL 1×TBE with 2.5 μL SYBR gold for 15 min). Aliquots of the 3.0 mL in vivo samples prepared above were withdrawn and a dilution series was prepared. For example, the sample (4.0 μL) was diluted with blue-dye loading buffer (6.0 μL) giving a sample with final RNA concentration of 0.04 μg/μL. Then 4 μL of this diluted sample was applied to the gel. Similarly, the sample (4.0 μL) was treated with DTT (1.0 μL, 1.0 M solution) for 10 minutes before being diluted further with 2.5% SDS (2.0 μL) and loading buffer (3.0 μL) giving a sample with final RNA concentration of 0.04 μg/μL. Then 4 μL of this reduced solution was also applied to the gel for analysis.

7-9 weeks old female Balb/C mice (Charles River Laboratories) were dosed with siRNA-polymer formulations via the tail vein at a dosing volume of 0.01 mL/g, n=5 per group. Final doses were as follows: 150 mg/kg polymer with 2 mg/kg ApoB siRNA; 75 mg/kg polymer with 1 mg/kg ApoB siRNA. Buffer solutions and solutions of non-targeted siRNA conjugates (ApoB siRNA conjugated to a diblock polymer b[HPMA-PDSMA]_{12.7 KDa}-b[D₂₅-B₅₀P₂₅]_{15.3 KDa} as described above) were used as the controls.

siRNA sequences for ApoB were as shown below:

Sense strand:

(SEQ ID NO: 1)
5′ OH—(CH₂)₆—S—S—(CH₂)₆-rGmUrCrAmUrCrArCrArCmUrGrArAmUrArCrCrArAmU-3′

Antisense strand:

(SEQ ID NO: 2)
5′-rArUrUrGrGrUrArUrUrCrArGrUrGrUrGrArUrGrArCrAr
C-3′

m=2′-O-methyl modified, r=ribonucleotide, d=deoxynucleotide

Animals were sacrificed 8-48 hours post dose. Blood was collected via cardiac puncture for serum isolation for blood chemistry analysis. Liver tissue was isolated (lower ⅔ of left lobe), placed in RNAlater (Ambion), and cut up in small pieces for improved tissue penetration. RNA was isolated from ˜50 mg of liver tissue using the MagMAx -96 for MicroArrays Total RNA Isolation kit (Ambion). RNA was converted to cDNA using the High Capacity cDNA Reverse Transcription Kit (Applied Biosystems). SYBR Green Real-Time PCR was performed to examine mRNA levels for siRNA target gene ApoB as well as normalize genes Calnexin 1 and Hprt1 (primer sequences are shown below).

| Name | Chemistry | Forward Primer: | Reverse Primer |
|---|---|---|---|
| Hprt1 | SYBR | 5′-CCTAAGATGAGCGCAAGTTGAA-3′ | 5′-CCACAGGACTAGAACACCTGCT-3′ |
| | | (SEQ ID NO: 3) | (SEQ ID NO: 4) |
| Calnexin | SYBR | 5′-ATGGAAGGGAAGTGGTTACTGT-3′ | 5′-GCTTTGTAGGTGACCTTTGGAG-3′ |
| | | (SEQ ID NO: 5) | (SEQ ID NO: 6) |
| ApoB | SYBR | 5′-AAGCACCTCCGAAAGTACGTG-3′ | 5′-CTCCAGCTCTACCTTACAGTTGA-3′ |
| | | (SEQ ID NO: 7) | (SEQ ID NO: 8) |

Step One Software v2.1 (Applied Biosystems) was used to normalize target genes to both normalizer genes. A cDNA aliquot from each buffer control sample was pooled and used to compare to each treated sample. In addition, each buffer control sample was compared to the pooled buffer samples.

The knockdown activity of the ApoB siRNA conjugates was 52% (dosed at 150 mg/kg polymer and 2 mg/kg siRNA, p=0.0069 compared to non-targeted control) and 41% (dosed at 75 mg/kg polymer and 1 mg/kg siRNA, p=0.075 compared to non-targeted control).

1. A composition comprising a block copolymer covalently conjugated to a polynucleotide, the block copolymer comprising a hydrophilic polymer block and a hydrophobic polymer block each comprising repeat units having chain atoms and pendant groups covalently coupled to the chain atoms, the polynucleotide being pendant and covalently coupled to the hydrophobic block, the hydrophobic block further comprising anionic repeat units having a population of pendant anions that varies in number in a pH dependant manner, the population being greater at pH 7.4 than at pH 5, wherein at least 90% of the repeat units of the hydrophilic and hydrophobic blocks are not the residues of amino acids linked by a peptidic bond.

2-13. (canceled)

14. The composition of claim 1 wherein the hydrophilic block comprises repeat units of Formula 1 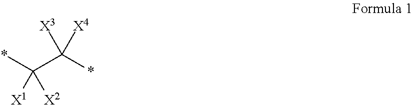
wherein
* designates the point of attachment of the repeat unit of Formula 1 to other repeat units;
each X¹ and X² is independently selected from the group consisting of hydrogen, hydrocarbyl, substituted hydrocarbyl, heterocyclo, and substituted carbonyl, provided that X¹ and X² are not, in the same repeat unit, selected from the group consisting of aryl, heteroaryl, heterosubstituted carbonyl, and combinations thereof;
each X³ is independently hydrogen, alkyl or substituted alkyl, and
each X⁴ is independently heterosubstituted carbonyl, aryl, or heteroaryl.

15-17. (canceled)

18. The composition of claim 14 wherein X⁴ is —C(O)OX⁴⁰ or —C(O)NX⁴⁰X⁴¹, and X⁴⁰ and X⁴¹ are independently hydrogen, hydrocarbyl, substituted hydrocarbyl, heterohydrocarbyl, substituted heterohydrocarbyl, or heterocyclo.

19. The composition of claim 14 wherein X¹ and X² are each hydrogen, X³ is hydrogen or alkyl, X⁴ is —C(O)OX⁴⁰, and X⁴⁰ is hydrogen, hydrocarbyl, substituted hydrocarbyl, heterohydrocarbyl, substituted heterohydrocarbyl, or heterocyclo.

20. The composition of claim 1 wherein the hydrophilic block comprises repeat units corresponding to Formula 1ETS: 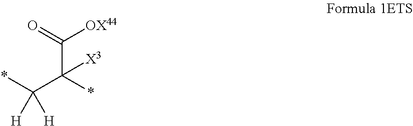
wherein * designates the point of attachment of the repeat unit of Formula 1ETS to other repeat units, X³ is alkyl, and X⁴⁴ is a targeting or shielding moiety.

21. The composition of claim 20 wherein X⁴⁴ is a polyol, vitamin, peptide or small molecule having a molecular weight of 200-1200 Daltons.

22. The composition of claim 1 wherein the hydrophilic block comprises repeat units derived from a polymerizable monomer having a formula 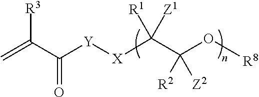
wherein:
n is an integer ranging from 2 to 20,
X is —(CR¹R²)m- wherein m is 0-10, and wherein one or more (CR¹R²) unit is optionally substituted with —NR¹—R², —OR¹ or —SR¹,
Y is —O—, —NR⁴— or —(CR¹R²)—,
each R¹, R², R³, Z¹ and Z² are independently selected from the group consisting of hydrogen, halogen, and optionally substituted C₁-C₃ alkyl,
R⁴ is selected from the group consisting of hydrogen, and optionally substituted C₁-C₆ alkyl,
R⁸ is hydrogen or (CR¹R²)ₘR⁹, wherein m is 0-10, and wherein one or more (CR¹R²) unit is optionally substituted with —NR¹R², —OR¹ or —SR¹, and
R⁹ is hydrogen, halogen, optionally substituted C₁-C₃ alkyl, polyol, vitamin, peptide or small molecule having a molecular weight of 200-1200 Daltons, or a conjugatable group.

23. The composition of claim 1 wherein the hydrophilic block comprises repeat units derived from a polymerizable monomer having a formula 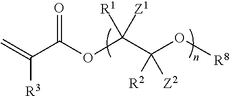
n is an integer ranging from 2 to 20,
each R¹, R², R³, Z¹ and Z² are independently selected from the group consisting of hydrogen, halogen, and optionally substituted C₁-C₃ alkyl,
R⁸ is hydrogen or (CR¹R²)ₘR⁹, wherein m is 0-10, and wherein one or more (CR¹R²) unit is optionally substituted with —NR¹R², —OR¹ or —SR¹, and
R⁹ is hydrogen, halogen, optionally substituted C₁-C₃ alkyl, polyol, vitamin, peptide or small molecule having a molecular weight of 200-1200 Daltons, or a conjugatable group.

24-28. (canceled)

29. The composition of claim 1 wherein the hydrophobic block comprise repeat units of Formula 1 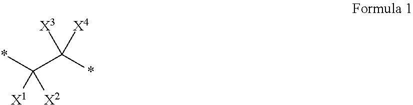
wherein
* designates the point of attachment of the repeat unit of Formula 1 to other repeat units;
each X¹ and X² is independently selected from the group consisting of hydrogen, hydrocarbyl, substituted hydrocarbyl, heterocyclo, and substituted carbonyl, provided that X¹ and X² are not, in the same repeat unit, selected from the group consisting of aryl, heteroaryl, heterosubstituted carbonyl, and combinations thereof;
each X³ is independently hydrogen, alkyl or substituted alkyl, and
each X⁴ is independently heterosubstituted carbonyl, aryl, or heteroaryl.

30-32. (canceled)

33. The composition of claim 29 wherein X⁴ is —C(O)OX⁴⁰, —C(O)SX⁴⁰, or —C(O)NX⁴⁰X⁴¹, and X⁴⁰ and X⁴¹ are independently hydrogen, hydrocarbyl, substituted hydrocarbyl, heterohydrocarbyl, substituted heterohydrocarbyl, or heterocyclo.

34-35. (canceled)

36. The composition of claim 29 wherein X¹ and X² are each hydrogen, X³ is hydrogen or alkyl, X⁴ is —C(O)OX⁴⁵, and X⁴⁵ is hydrogen, hydrocarbyl, substituted hydrocarbyl, heterohydrocarbyl, substituted heterohydrocarbyl, or heterocyclo.

37. The composition of claim 29 wherein X¹ and X² are each hydrogen, X³ is hydrogen or alkyl, X⁴ is —C(O)OX⁴⁵, X⁴⁵ is a disulfide substituted alkyl moiety.

38. The composition of claim 37 wherein X⁴⁵ is pyridyl disulfide substituted ethyl.

39. (canceled)

40. The composition of claim 1 wherein the hydrophobic block comprises repeat units corresponding to Formula 1A: 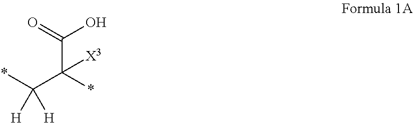
wherein * designates the point of attachment of the repeat unit of Formula 1A to other repeat units, and X³ is alkyl.

41. The composition of claim 1 wherein the hydrophobic block comprises repeat units corresponding to Formula 1E: 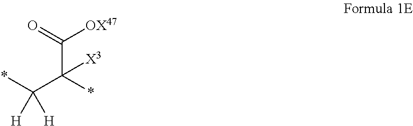
wherein * designates the point of attachment of the repeat unit of Formula 1E to other repeat units, and X³ and X⁴⁷ are independently alkyl.

42. The composition of claim 1 wherein the hydrophobic block comprises repeat units corresponding to Formulae 1A and 1E 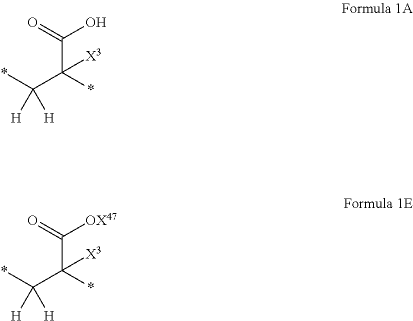
wherein * designates the point of attachment of the repeat unit of Formula 1A and 1E to other repeat units, each X³ and X⁴⁷ are independently alkyl, and the ratio of the number of repeat units corresponding to Formula 1A to the number of repeat units corresponding to Formula 1E is between about 20:1 and 1:4, respectively.

43. The composition of claim 1 wherein the hydrophobic block comprises repeat units corresponding to Formula 1C: 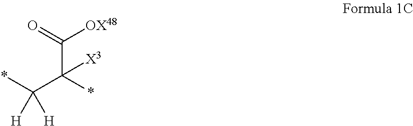
wherein * designates the point of attachment of the repeat unit of Formula 1C to other repeat units, X³ is alkyl, and X⁴⁸ is amino-substituted alkyl.

44. The composition of claim 1 wherein the hydrophobic block comprises repeat units corresponding to Formulae 1A and 1C 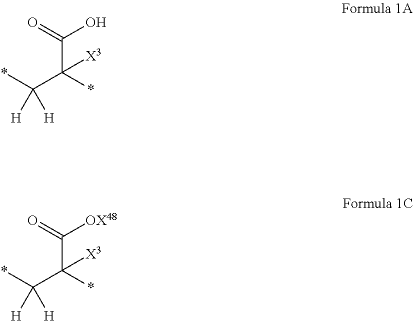
wherein * designates the point of attachment of the repeat unit of Formula 1A and 1C to other repeat units, each X³ is independently alkyl, X⁴⁸ is amino-substituted alkyl, and the number of repeat units corresponding to Formula 1A is at least equal to the number of repeat units corresponding to Formula 1C.

45-46. (canceled)

47. The composition of claim 1 wherein the hydrophobic block comprises repeat units corresponding to Formula 1-CON 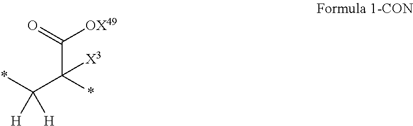
wherein * designates the point of attachment of the repeat unit of Formula 1-CON to other repeat units; X³ is hydrogen or alkyl, and X⁴⁹ comprises a conjugatable group.

48. The composition of claim 47 wherein X⁴⁹ is substituted hydrocarbyl, heterohydrocarbyl, or heterocyclo.

49. The composition of claim 47 wherein X⁴⁹ is an alkyl group substituted by N-hydroxysuccinimide (NHS)ester, HOBt (1-hydroxybenzotriazole) ester, p-nitrophenyl ester, tetrafluorophenyl ester, pentafluorophenyl ester, pyridyl disulfide group, maleimide, aldehyde, ketone, anhydride, thiol, amine, hydroxyl or alkyl halide.

50-51. (canceled)

52. The composition of claim 1 wherein hydrophobic block comprises, as repeat units, the residues of 2-propylacrylic acid, N,N-dimethylaminoethyl methacrylate, butyl methacrylate, and pyridyldisulfide methacrylate ester.

53-58. (canceled)

59. The composition of claim 1 wherein the polynucleotide is an siRNA, an antisense oligonucleotide, a dicer substrate, an miRNA, an aiRNA or an shRNA.

60-69. (canceled)

70. A pharmaceutical composition comprising the composition of claim 1 and a pharmaceutically acceptable excipient.

71. A method for intracellular delivery of a polynucleotide, the method comprising contacting a composition of claim 1 with a cell surface in a medium at a first pH; introducing the composition into an endosomal membrane within the cell through endocytosis; and destabilizing the endosomal membrane, whereby the composition or the polynucleotide is delivered to the cytosol of the cell.

72. (canceled)
